(19) United States
(12) Patent Application Publication
(10) Pub. No.: US 2023/0407291 A1
(43) Pub. Date: Dec. 21, 2023

(54) TARGETING HOST-BACTERIA INTERACTIONS FOR THE TREATMENT OF MICROBIOTA-MEDIATED DISEASES

(71) Applicant: YALE UNIVERSITY, New Haven, CT (US)

(72) Inventors: Connor Rosen, New Haven, CT (US); Noah Palm, New Haven, CT (US); Aaron Ring, New Haven, CT (US)

(21) Appl. No.: 18/253,250

(22) PCT Filed: Nov. 17, 2021
(86) PCT No.: PCT/US21/59643

§ 371 Date: May 17, 2023

Related U.S. Application Data
(60) Provisional application No. 63/114,627, filed on Nov. 17, 2020.

(51) Int. Cl. C12N 15/10

(52) U.S. Cl. CPC C12N 15/1037

(57) ABSTRACT

The present invention generally relates to compositions and methods for modulating specific paired host protein-microbiota interactions and the use thereof for the prevention and treatment of diseases and disorders.

### CROSS REFERENCE TO RELATED APPLICATIONS

This application claims priority to U.S. Provisional Application No. 63/114,627, filed Nov. 17, 2020 which is hereby incorporated by reference herein in its entirety.

### BACKGROUND OF THE INVENTION

Direct interactions with host proteins are key factors in microbial infections and provide insight into mechanisms of bacterial pathogenesis. By contrast, the collective understanding of the molecular mechanisms by which host-associated microbial communities (microbiotas) interact with host proteins is comparatively limited. This is despite widespread evidence of close physical association between select members of the microbiota and host tissues and the profound effects of the microbiome on host physiology. A limitation to better understanding direct host-microbe interactions is the paucity of systems and methods for high-throughput screening of microbial binding by host extracellular proteins—those most likely to come into contact with microbes.

Thus, there is a need in the art for improved systems and high-throughput methods for identifying microbial interactions with specific host proteins that would enable mapping of host-microbiota interactions at scale. This invention satisfies this unmet need.

### SUMMARY OF THE INVENTION

In one embodiment, the invention relates to a composition for modulating an interaction of a host protein with an associated microbial cell, wherein the host protein and microbial cell are interacting partners selected from the group consisting of host protein and microbial cell interacting partners as set forth in Table 1.

In one embodiment, the modulator is an inhibitor of the host protein selected from the group consisting of the host protein listed in Table 1.

In one embodiment, the inhibitor is a small interfering RNA (siRNA), a microRNA, an antisense nucleic acid, a ribozyme, an expression vector encoding a transdominant negative mutant, an antibody, an antibody fragment, a peptide, a chemical compound or a small molecule.

In one embodiment, the modulator is an activator of a host protein listed in Table 1. In one embodiment, the activator increases one or more of transcription and translation of the host protein listed in Table 1.

In one embodiment, the activator is a nucleic acid, a protein, a peptide, a peptidomemetic, a chemical compound or a small molecule.

In one embodiment, the interaction of the host protein with the associated microbial cell is the interaction of Fusobacterium with an immune-modulatory protein. In one embodiment, the immune-modulatory protein is AGER, BTN3A3, BTNL8, C3AR, CCL5, CCR9, CD55, CD99L2, CEACAM4, CSF3, DKK1, DKK2, IL15RA, LAIR1, MADCAM1, MCAM, MERTK, NPY5R, SIRPA, SOST, TMEM149, TNFRSF10B, TNFRSF4, or TREML1. In one embodiment, the interaction of the host protein with the associated microbial cell is the interaction of Ruminococcus gnavus with CD7, TFF1, TFF2, or TFF3.

In one embodiment, the invention relates to a method of modulating an interaction of a host protein with an associated microbial cell, wherein the host protein and microbial cell are interacting partners selected from the group consisting of host protein and microbial cell interacting partners as set forth in Table 1, the method comprising contacting a host cell with a composition for modulating the interaction of the host protein and the microbial cell.

In one embodiment, the modulator is an inhibitor of a host protein listed in Table 1. In one embodiment, the inhibitor is a small interfering RNA (siRNA), a microRNA, an antisense nucleic acid, a ribozyme, an expression vector encoding a transdominant negative mutant, an antibody, an antibody fragment, a peptide, a chemical compound or a small molecule.

In one embodiment, the modulator is an activator a host protein listed in Table 1. In one embodiment, the activator increases one or more of transcription and translation of a host protein listed in Table 1. In one embodiment, the activator is a nucleic acid, a protein, a peptide, a peptidomemetic, a chemical compound or a small molecule.

In one embodiment, the invention relates to a method of treating or preventing a disease or disorder in a subject in need thereof, the method comprising administering a composition for modulating the interaction of a host protein and a microbial cell to the subject, wherein the host protein and microbial cell are interacting partners selected from the group consisting of host protein and microbial cell interacting partners as set forth in Table 1.

In one embodiment, the modulator is an inhibitor of the host protein selected from the group consisting of the host protein listed in Table 1.

In one embodiment, the inhibitor is a small interfering RNA (siRNA), a microRNA, an antisense nucleic acid, a ribozyme, an expression vector encoding a transdominant negative mutant, an antibody, an antibody fragment, a peptide, a chemical compound or a small molecule.

In one embodiment, the modulator is an activator of a host protein listed in Table 1. In one embodiment, the activator increases one or more of transcription and translation of a host protein listed in Table 1.

In one embodiment, the activator is a nucleic acid, a protein, a peptide, a peptidomemetic, a chemical compound or a small molecule.

In one embodiment, the disease or disorder is inflammatory diseases, inflammatory bowel disease (IBD), irritable bowel syndrome (IBS), obesity, type 2 diabetes, cardiovascular disease, Alzheimer's disease, Parkinson's disease, cancer or atopy.

### BRIEF DESCRIPTION OF THE DRAWINGS

The following detailed description of embodiments of the invention will be better understood when read in conjunction with the appended drawings. It should be understood that the invention is not limited to the precise arrangements and instrumentalities of the embodiments shown in the drawings.

FIG. 1 depicts a diagram demonstrating that BASEHIT profiling enables screening of hundreds of isolates representing a broad sample of the human microbiome against the human exoproteome to identify global patterns as well as highlight individual bacterial activities.

FIG. 2 depicts exemplary results demonstrating heatmap visualization of all binary interactions between 519 screened strains and 3336 host proteins.

FIG. 3A and FIG. 3B depict exemplary results demonstrating orthogonal validations of newly identified host-microbe interactions by flow cytometry (FIG. 3A) or ELISA (FIG. 3D). Staining with indicated Fc-fusion proteins (red, green, blue) is compared to Fc-control protein (black).

FIG. 4 depicts exemplary results demonstrating that host-microbe interaction patterns vary substantially across the collective human microbiome. Bars represent the tissue of origin for microbial strains (left column), phylum (middle column), and number of detected interactions with host proteins (right). Flows between bars represent the fraction of strains sharing the properties of the two connected bars.

FIG. 5A through FIG. 5B depict exemplary results demonstrating that host-specific interactions vary across closely related strains of microbes. FIG. 5A depicts exemplary results demonstrating a bar plot of interactions with skin-expressed proteins across Staphylococcus isolates. Each column represents a single strain and is colored according to species. Bars above the x-axis represent hits. FIG. 5B depicts the network of interactions between Staphylococcus strains from various species and host proteins. Strains and proteins, represented as differently colored circles, are connected by a line if there is a detected interaction between that particular strain/protein pair.

FIG. 6A depicts exemplary results demonstrating that staining of R. gnavus strains NWP327 (red), 325 (blue), and 326 (black) show strain-level variability in CD7 binding.

FIG. 6B depicts exemplary results demonstrating that staining of R. gnavus NWP327 with increasing concentrations of human or mouse CD7 protein shows species specificity in host receptor binding.

FIG. 7 depicts exemplary results demonstrating interactions of Fusobacterium strains with tissue and immune proteins. Each strain is a row, while each protein is a column. If an interaction was detected between a particular strain/protein pair, a grey circle is shown.

### DETAILED DESCRIPTION

The present invention relates to systems and methods for large-scale, high-throughput mapping of host protein-microbiota interactions. The present invention is based, in part, on the optimization and validation of the BASEHIT technique and, in part, on the identification of novel host protein-microbiota interactions identified in Table 1. In some embodiments, invention provides composition and methods for modulating host protein-microbe interactions. In some embodiments, the host protein-microbe interaction is modulated to alleviate symptoms of a disease or disorder mediated by said interactions. In some embodiments, the invention provides methods of inhibiting one or more host protein-microbe interaction. In some embodiments, the invention provides methods of promoting one or more host protein-microbe interaction.

### Definitions

Unless defined otherwise, all technical and scientific terms used herein have the same meaning as commonly understood by one of ordinary skill in the art to which this invention belongs.

As used herein, each of the following terms has the meaning associated with it in this section.

The articles “a” and “an” are used herein to refer to one or to more than one (i.e., to at least one) of the grammatical object of the article. By way of example, “an element” means one element or more than one element.

“About” as used herein when referring to a measurable value such as an amount, a temporal duration, and the like, is meant to encompass variations of ±20%, ±10%, ±5%, ±1%, or ±0.1% from the specified value, as such variations are appropriate to perform the disclosed methods.

The term “activate,” as used herein, means to induce or increase an activity or function, for example, about ten percent relative to a control value. Preferably, the activity is induced or increased by 50% compared to a control value, more preferably by 75%, and even more preferably by 95%. “Activate,” as used herein, also means to increase a molecule, a reaction, an interaction, a gene, an mRNA, and/or a protein's expression, stability, function or activity by a measurable amount or to increase entirely. Activators are compounds that, e.g., bind to, partially or totally induce stimulation, increase, promote, induce activation, activate, sensitize, or up regulate a protein, a gene, and an mRNA stability, expression, function and activity, e.g., agonists.

As used herein in reference to a display library, a “barcode” refers to a unique molecular identifier to distinguish cells expressing distinct display molecules. For example, the barcode may be a unique DNA sequence within a cell that corresponds to a display molecule expressed by said cell. This barcode may be detected using methods including, but not limited to, next generation sequencing. As used herein the term “cell surface molecule” refers to a peptide, polypeptide, binding domain, ligand, lipid, or carbohydrate that is directed to the extracellular surface of the host cell. The cell surface molecule may be anchored to the cell surface by covalent binding or non-covalent binding. The cell surface molecule may include a phospholipid, carbohydrate, or protein through which it attaches to the surface of the host cell. The cell surface molecule may be a polypeptide that binds to, or is conjugated to, a phospholipid, carbohydrate, or a polypeptide on the surface of the cell. For example, the polypeptide may use a phosphatidyl-inositol-glycan (GPI) anchor to attach to the surface of the cell, such as a-agglutinins, α-agglutinins, and flocculins. The cell surface molecule may also be a transmembrane protein.

“Coding sequence” or “encoding nucleic acid” as used herein may refer to the nucleic acid (RNA or DNA molecule) that comprise a nucleotide sequence which encodes an antigen set forth herein. The coding sequence may further include initiation and termination signals operably linked to regulatory elements including a promoter and polyadenylation signal capable of directing expression in the one or more cells of an individual or mammal to whom the nucleic acid is administered. The coding sequence may further include sequences that encode signal peptides.

A “constitutive” promoter is a nucleotide sequence which, when operably linked with a polynucleotide which encodes or specifies a gene product, causes the gene product to be produced in a cell under most or all physiological conditions of the cell.

A “disease” is a state of health of an animal wherein the animal cannot maintain homeostasis, and wherein if the disease is not ameliorated then the animal's health continues to deteriorate. In contrast, a “disorder” in an animal is a state of health in which the animal is able to maintain homeostasis, but in which the animal's state of health is less favorable than it would be in the absence of the disorder. Left untreated, a disorder does not necessarily cause a further decrease in the animal's state of health.

A disease or disorder is “alleviated” if the severity of a sign or symptom of the disease, or disorder, the frequency with which such a sign or symptom is experienced by a patient, or both, is reduced.

As used herein the term “display molecule” refers to a molecule that can be localized to the surface of a target cell. The display molecule will typically comprise a first amino acid sequence to be displayed (e.g., a protein of interest, etc.) and a second amino acid sequence that anchors the display molecule to the surface of the target cell (e.g., a transmembrane domain, etc.). In certain instances, the first and second amino acid sequences are linked in a single polypeptide. In an alternative embodiment, the first and second amino acid sequences may interact with each other to anchor the first amino acid sequence to the surface of a target cell. A display molecule may comprise a peptide, polypeptide, binding domain, ligand, lipid, or carbohydrate or combination thereof. The display molecule may also comprise a tag or peptide that can be labeled so as to detect binding of the display molecule to the cell surface, or sort cells displaying said molecule.

As used herein the term “display library” refers to a plurality of cells, wherein each cell comprises a non-identical display molecule that is displayed on the surface of the cell.

As used herein the term “enrich” or “enrichment” refers to the state or process, respectively, of increasing the proportion of a species of interest within a mixed pool of species through some form of selection. For example, a protein of interest can be enriched from a mixed sample of multiple proteins using positive selection with a protein-specific antibody.

The term “expression” as used herein is defined as the transcription of a particular nucleotide sequence driven by its promoter and/or the translation of said nucleotide sequence into an amino acid sequence.

An “expression cassette” is a nucleic acid construct, generated recombinantly or synthetically, with a series of specified nucleic acid elements that permit transcription of a particular polynucleotide sequence in a host cell. An expression cassette may be part of a plasmid, viral genome, or nucleic acid fragment. Typically, an expression cassette includes a polynucleotide to be transcribed, operably linked to a promoter.

The term “gene” means the segment of DNA involved in producing a polypeptide chain. It may include regions preceding and following the coding region (leader and trailer) as well as intervening sequences (introns) between individual coding segments (exons).

As used herein, an “inducible” promoter is a nucleotide sequence which, when operably linked with a polynucleotide which encodes or specifies a gene product, causes the gene product to be produced substantially only when an inducer which corresponds to the promoter is present.

The term “inhibit,” as used herein, means to suppress or block an activity or function, for example, about ten percent relative to a control value. Preferably, the activity is suppressed or blocked by 50% compared to a control value, more preferably by 75%, and even more preferably by 95%. “Inhibit,” as used herein, also means to reduce a molecule, a reaction, an interaction, a gene, an mRNA, and/or a protein's expression, stability, function or activity by a measurable amount or to prevent entirely. Inhibitors are compounds that, e.g., bind to, partially or totally block stimulation, decrease, prevent, delay activation, inactivate, desensitize, or down regulate a protein, a gene, and an mRNA stability, expression, function and activity, e.g., antagonists.

As used herein, an “instructional material” includes a publication, a recording, a diagram, or any other medium of expression which can be used to communicate the usefulness of a compound, composition, vector, or delivery system of the invention in the kit for effecting alleviation of the various diseases or disorders recited herein. Optionally, or alternately, the instructional material can describe one or more methods of alleviating the diseases or disorders in a cell or a tissue of a mammal. The instructional material of the kit of the invention can, for example, be affixed to a container which contains the identified compound, composition, vector, or delivery system of the invention or be shipped together with a container which contains the identified compound, composition, vector, or delivery system. Alternatively, the instructional material can be shipped separately from the container with the intention that the instructional material and the compound be used cooperatively by the recipient.

“Measuring” or “measurement,” or alternatively “detecting” or “detection,” means assessing the presence, absence, quantity or amount (which can be an effective amount) of a given substance.

The term “modulate,” as used herein, refers to mediating a detectable increase or decrease in a desired response. For example, a small molecule may be used to increase or decrease the level of interaction between two proteins.

As used herein, the term “next generation sequencing” refers to sequencing methods that allow for massively parallel sequencing of clonally amplified molecules and of single nucleic acid molecules. Next generation sequencing is synonymous with “massively parallel sequencing” for most purposes. Non-limiting examples of next generation sequencing include sequencing-by-synthesis using reversible dye terminators, and sequencing-by-ligation.

The term “nucleic acid” or “polynucleotide” refers to deoxyribonucleic acids (DNA) or ribonucleic acids (RNA) and polymers thereof in either single- or double-stranded form. Unless specifically limited, the term encompasses nucleic acids containing known analogues of natural nucleotides that have similar binding properties as the reference nucleic acid and are metabolized in a manner similar to naturally occurring nucleotides. Unless otherwise indicated, a particular nucleic acid sequence also implicitly encompasses conservatively modified variants thereof (e.g., degenerate codon substitutions), alleles, orthologs, SNPs, and complementary sequences as well as the sequence explicitly indicated. Specifically, degenerate codon substitutions may be achieved by generating sequences in which the third position of one or more selected (or all) codons is substituted with mixed-base and/or deoxyinosine residues (Batzer et al., Nucleic Acid Res. 19:5081 (1991); Ohtsuka et al., J. Biol. Chem. 260:2605-2608 (1985); and Rossolini et al, Mol. Cell. Probes 8:91-98 (1994)). The term nucleic acid is used interchangeably with gene, cDNA, and mRNA encoded by a gene.

“Operably linked” as used herein may mean that expression of a gene is under the control of a promoter with which it is spatially connected. A promoter may be positioned 5′ (upstream) or 3′ (downstream) of a gene under its control. The distance between the promoter and a gene may be approximately the same as the distance between that promoter and the gene it controls in the gene from which the promoter is derived. As is known in the art, variation in this distance may be accommodated without loss of promoter function.

As used herein, a “plurality of cells” herein is meant roughly from about 10³ cells to 10⁸ or 10⁹, with from 10⁶ to 10⁸ being common.

As used herein, the term “plurality of display molecules” refers to at least two copies of a display molecule displayed on the surface of a target cell. In certain instances, each unique display molecule is displayed by a different target cell.

As used herein in reference to interactions, “promote” refers to inducing or increasing an interaction between two species. For example, a small molecule may promote or increase interactions between two proteins.

“Promoter” as used herein may mean a synthetic or naturally-derived molecule which is capable of conferring, activating or enhancing expression of a nucleic acid in a cell. A promoter may comprise one or more specific transcriptional regulatory sequences to further enhance expression and/or to alter the spatial expression and/or temporal expression of same. A promoter may also comprise distal enhancer or repressor elements, which can be located as much as several thousand base pairs from the start site of transcription. A promoter may be derived from sources including viral, bacterial, fungal, plants, insects, and animals. A promoter may regulate the expression of a gene component constitutively, or differentially with respect to cell, the tissue or organ in which expression occurs or, with respect to the developmental stage at which expression occurs, or in response to external stimuli such as physiological stresses, pathogens, metal ions, or inducing agents. Representative examples of promoters include the promoters from GAL1 (galactose), PGK (phosphoglycerate kinase), ADH (alcohol dehydrogenase), AOX1 (alcohol oxidase), HIS4 (histidinol dehydrogenase), metallothionein, 3-phosphoglycerate kinase, such as enolase, glyceraldehyde-3-phosphate dehydrogenase, hexokinase, pyruvate decarboxylase, phospho-fructokinase, glucose-6-phosphate isomerase, 3-phosphoglycerate mutase, pyruvate kinase, triosephosphate isomerase, phospho-glucose isomerase, and glucokinase.

The term “regulating” as used herein can mean any method of altering the level or activity of a substrate. Non-limiting examples of regulating with regard to a protein include affecting expression (including transcription and/or translation), affecting folding, affecting degradation or protein turnover, and affecting localization of a protein. Non-limiting examples of regulating with regard to an enzyme further include affecting the enzymatic activity. “Regulator” refers to a molecule whose activity includes affecting the level or activity of a substrate. A regulator can be direct or indirect. A regulator can function to activate or inhibit or otherwise modulate its substrate.

The terms “subject”, “individual”, “patient” and the like are used interchangeably herein, and refer to any animal, or cells thereof whether in vitro or in situ, amenable to the methods described herein. In some non-limiting embodiments, the patient, subject or individual is a human. In various embodiments, the subject is a human subject, and may be of any race, sex, and age.

“Vector” as used herein may mean a nucleic acid sequence containing an origin of replication. A vector may be a plasmid, bacteriophage, bacterial artificial chromosome or yeast artificial chromosome. A vector may be a DNA or RNA vector. A vector may be either a self-replicating extrachromosomal vector or a vector which integrates into a host genome.

As used herein, the term “wild-type” refers to a gene or gene product isolated from a naturally occurring source. A wild-type gene is that which is most frequently observed in a population and is thus arbitrarily designed the “normal” or “wild-type” form of the gene. In contrast, the term “modified” or “mutant” refers to a gene or gene product that displays modifications in sequence and/or functional properties (i.e., altered characteristics) when compared to the wild-type gene or gene product. It is noted that naturally occurring mutants can be isolated; these are identified by the fact that they have altered characteristics (including altered nucleic acid sequences) when compared to the wild-type gene or gene product

Ranges: throughout this disclosure, various aspects of the invention can be presented in a range format. It should be understood that the description in range format is merely for convenience and brevity and should not be construed as an inflexible limitation on the scope of the invention. Accordingly, the description of a range should be considered to have specifically disclosed all the possible subranges as well as individual numerical values within that range. For example, description of a range such as from 1 to 6 should be considered to have specifically disclosed subranges such as from 1 to 3, from 1 to 4, from 1 to 5, from 2 to 4, from 2 to 6, from 3 to 6 etc., as well as individual numbers within that range, for example, 1, 2, 2.7, 3, 4, 5, 5.3, and 6. This applies regardless of the breadth of the range.

### DESCRIPTION

In one embodiment, the method relates to the identification of direct binding interactions between a host protein and a microbial cell using BASEHIT (described in WO2018208877, incorporated herein by reference). In some embodiments the invention relates to a composition for modulating the interaction of one or more host protein and a microbial cell, and methods of use thereof. Modulating the interaction of a host protein and a microbial cell can be done through a variety of well-understood means, including, but not limited to, antibodies, inhibitory nucleic acid molecules (e.g., siRNA), small molecules, proteins, drug modalities or by targeting individual bacteria that have an effect on the host.

In various embodiments, the compositions of the invention include modulators of host proteins, modulators of microbial cells, and modulators of the interaction between a host protein and a microbial cell. In some embodiments, the host protein is selected from a protein identified in Table 1. In some embodiments, the microbial cell is selected from a strain identified in Table 1.

Exemplary specific interactions that are included in Table 1, and can be modulated according to the invention include, but are not limited to, the interaction of a Streptococcus strain with BACE2, a protease involved in Alzheimer's disease; interactions between strains of Fusobacterium isolated from colon tumors and immune-modulatory proteins; and interactions between Ruminococcus gnavus strains isolated from IBD patients and proteins expressed on T cells, including CD7 and members of the TNF receptor superfamily including, but not limited to, TMEM149, TNFRSF1B, TNFRSF4, TNFRSF7, TNFRSF9. Therefore, in one embodiment, the invention includes compositions and methods for modulating the interaction of Streptococcus with BACE2. In one embodiment, the invention includes compositions and methods for modulating the interaction of Fusobacterium and immune-modulatory proteins including, but not limited to, AGER, BTN3A3, BTNL8, C3AR, CCL5, CCR9, CD55, CD99L2, CEACAM4, CSF3, DKK1, DKK2, IL15RA, LAIR1, MADCAM1, MCAM, MERTK, NPY5R, SIRPA, SOST, TMEM149, TNFRSF10B, TNFRSF4, and TREML1. In one embodiment, the invention includes compositions and methods for modulating the interaction of Ruminococcus gnavus and proteins expressed on T cells, including CD7 and members of the TNF receptor superfamily including, but not limited to, CD7, TFF1, TFF2, and TFF3. In one embodiment, the invention includes compositions and methods for modulating the interaction of Citrobacter and a fibroblast growth factor (FGF) including, but not limited to, FGF1 and FGF7.

### Activators

In various embodiments, the present invention includes compositions and methods of modulating a host protein, or the interaction of a host protein with a microbial cell, or a combination thereof, to activate or increase the level of interaction between the host protein and the microbial cell.

Therefore, in various embodiments, the composition for modulating a host protein, or the interaction of a host protein with a microbial cell, or a combination thereof, increases the amount of host protein polypeptide, the amount of host protein mRNA, the amount of host protein activity, or a combination thereof.

It will be understood by one skilled in the art, based upon the disclosure provided herein, that an increase in the level of the host protein encompasses the increase in host protein expression, including transcription, translation, or both. The skilled artisan will also appreciate, once armed with the teachings of the present invention, that an increase in the level of the host protein includes an increase in host protein activity. Thus, increasing the level or activity of a host protein includes, but is not limited to, increasing the amount of the host protein polypeptide, increasing transcription, translation, or both, of a nucleic acid encoding the host protein; and it also includes increasing any activity of a host protein polypeptide as well, such that the activity results in an increase in interaction of the host protein with a microbial cell.

In some embodiments, the present invention relates to the prevention and treatment of a disease or disorder by administration of a host protein polypeptide, a recombinant host protein polypeptide, an active host protein polypeptide fragment, or an activator of host protein expression or activity.

Activation of a host protein can be assessed using a wide variety of methods, including those disclosed herein, as well as methods well-known in the art or to be developed in the future. That is, a person of skill in the art would appreciate, based upon the disclosure provided herein, that increasing the level or activity of a host protein can be readily assessed using methods that assess the level of a nucleic acid encoding the host protein (e.g., mRNA) and/or the level of host protein polypeptide in a biological sample obtained from a subject.

A host protein activator can include, but should not be construed as being limited to, a chemical compound, a protein, a peptidomemetic, an antibody, and a nucleic acid molecule. One of skill in the art would readily appreciate, based on the disclosure provided herein, that a host protein activator encompasses a chemical compound that increases the level, activity, or the like of host protein. Additionally, a host protein activator encompasses a chemically modified compound, and derivatives, as is well known to one of skill in the chemical arts.

Further, one of skill in the art would, when equipped with this disclosure and the methods exemplified herein, appreciate that a host protein activator includes such activators as discovered in the future, as can be identified by well-known criteria in the art of pharmacology, such as the physiological results of activation of host protein as described in detail herein and/or as known in the art. Therefore, the present invention is not limited in any way to any particular host protein activator as exemplified or disclosed herein; rather, the invention encompasses those activators that would be understood by one of skill in the art to be useful, as are known in the art, and as are discovered in the future.

Further methods of identifying and producing a host protein activator are well known to those of ordinary skill in the art, including, but not limited, obtaining an activator from a naturally occurring source. Alternatively, a host protein activator can be synthesized chemically. Further, one of skill in the art would appreciate, based upon the teachings provided herein, that a host protein activator can be obtained from a recombinant organism. Compositions and methods for chemically synthesizing host protein activators and for obtaining them from natural sources are well known in the art and are described in the art.

One of skill in the art will appreciate that an activator can be administered as a small molecule chemical, a protein, a nucleic acid construct encoding a protein, or combinations thereof. Numerous vectors and other compositions and methods are well known for administering a protein or a nucleic acid construct encoding a protein to cells or tissues. Therefore, the invention includes a method of administering a protein or a nucleic acid encoding a protein that is an activator of host protein.

One of skill in the art will realize that diminishing the amount or activity of a molecule that itself diminishes the amount or activity of host protein can serve to increase the amount or activity of host protein. Exemplary inhibitory compositions include, but are not limited to, antisense oligonucleotides, antibodies, small molecule chemical compounds and other inhibitory compositions as discussed elsewhere herein. Any inhibitor of a regulator of host protein is encompassed in the invention.

In some embodiments, the host protein activator compositions and methods of the invention can selectively activate the host protein. Thus, in various embodiments, the present invention relates to compositions comprising a host protein polypeptide, a recombinant host protein polypeptide, or an active host protein polypeptide fragment.

One of skill in the art will appreciate that a host protein polypeptide, a recombinant host protein polypeptide, or an active host protein polypeptide fragment can be administered singly or in any combination thereof. Further, a host protein polypeptide, a recombinant host protein polypeptide, or an active host protein polypeptide fragment can be administered singly or in any combination thereof in a temporal sense, in that they may be administered simultaneously, before, and/or after each other. One of ordinary skill in the art will appreciate, based on the disclosure provided herein, that a host protein polypeptide, a recombinant host protein polypeptide, or an active host protein polypeptide fragment can be used to prevent or treat a disease or disorder, and that an activator can be used alone or in any combination with another host protein polypeptide, recombinant host protein polypeptide, active host protein polypeptide fragment, or host protein activator to effect a therapeutic result.

One of skill in the art, when armed with the disclosure herein, would appreciate that the treating or preventing a disease or disorder encompasses administering to a subject a host protein polypeptide, a recombinant host protein polypeptide, an active host protein polypeptide fragment, or host protein activator as a preventative measure against a neurodegenerative disease or disorder. As more fully discussed elsewhere herein, methods of increasing the level or activity of a host protein encompass a wide plethora of techniques for increasing not only host protein activity, but also for increasing expression of a nucleic acid encoding host protein. Additionally, as disclosed elsewhere herein, one skilled in the art would understand, once armed with the teaching provided herein, that the present invention encompasses a method of preventing a wide variety of diseases where increased expression and/or interaction of a host protein with a microbial cell mediates, treats or prevents the disease. Further, the invention encompasses treatment or prevention of such diseases discovered in the future.

The invention encompasses administration of a host protein polypeptide, a recombinant host protein polypeptide, an active host protein polypeptide fragment, or a host protein activator to practice the methods of the invention; the skilled artisan would understand, based on the disclosure provided herein, how to formulate and administer the appropriate host protein polypeptide, recombinant host protein polypeptide, active host protein polypeptide fragment, or host protein activator to a subject. However, the present invention is not limited to any particular method of administration or treatment regimen. This is especially true where it would be appreciated by one skilled in the art, equipped with the disclosure provided herein, including the reduction to practice using an art-recognized model of a neurodegenerative disease, that methods of administering a host protein polypeptide, a recombinant host protein polypeptide, an active host protein polypeptide fragment, or host protein activator can be determined by one of skill in the pharmacological arts.

As used herein, the term “pharmaceutically-acceptable carrier” means a chemical composition with which an appropriate host protein polypeptide, recombinant host protein polypeptide, active host protein polypeptide fragment, or host protein activator, may be combined and which, following the combination, can be used to administer the appropriate host protein polypeptide, recombinant host protein polypeptide, active host protein polypeptide fragment, or host protein activator to a subject.

### Inhibitors

In various embodiments, the present invention includes compositions and methods of modulating a host protein, or the interaction of a host protein with a microbial cell, or a combination thereof, which inhibit or decrease the level of interaction between the host protein and the microbial cell.

Therefore, in various embodiments, the composition for modulating a host protein, or the interaction of a host protein with a microbial cell, or a combination thereof, decreases the amount of host protein polypeptide, the amount of host protein mRNA, the amount of host protein activity, or a combination thereof.

It will be understood by one skilled in the art, based upon the disclosure provided herein, that a decrease in the level of a host protein encompasses the decrease in the expression, including transcription, translation, or both. The skilled artisan will also appreciate, once armed with the teachings of the present invention, that a decrease in the level of the host protein includes a decrease in the activity of host protein. Thus, a decrease in the level or activity of host protein includes, but is not limited to, decreasing the amount of polypeptide of the host protein, and decreasing transcription, translation, or both, of a nucleic acid encoding the host protein; and it also includes decreasing any activity of host protein as well, wherein the activity is associated with the interaction of the host protein and a microbial cell.

In one embodiment, the composition of the invention comprises an inhibitor of host protein. In one embodiment, the inhibitor is selected from the group consisting of a small interfering RNA (siRNA), a microRNA, an antisense nucleic acid, a ribozyme, an expression vector encoding a transdominant negative mutant, an antibody, a peptide and a small molecule.

One skilled in the art will appreciate, based on the disclosure provided herein, that one way to decrease the mRNA and/or protein levels of a host protein in a cell is by reducing or inhibiting expression of the nucleic acid encoding the host protein. Thus, the protein level of the host protein in a cell can be decreased using a molecule or compound that inhibits or reduces gene expression such as, for example, siRNA, an antisense molecule or a ribozyme. However, the invention should not be limited to these examples.

In one embodiment, siRNA is used to decrease the level of host protein. RNA interference (RNAi) is a phenomenon in which the introduction of double-stranded RNA (dsRNA) into a diverse range of organisms and cell types causes degradation of the complementary mRNA. In the cell, long dsRNAs are cleaved into short 21-25 nucleotide small interfering RNAs, or siRNAs, by a ribonuclease known as Dicer. The siRNAs subsequently assemble with protein components into an RNA-induced silencing complex (RISC), unwinding in the process. Activated RISC then binds to complementary transcript by base pairing interactions between the siRNA antisense strand and the mRNA. The bound mRNA is cleaved and sequence specific degradation of mRNA results in gene silencing. See, for example, U.S. Pat. No. 6,506,559; Fire et al., 1998, Nature 391(19):306-311; Timmons et al., 1998, Nature 395:854; Montgomery et al., 1998, TIG 14 (7):255-258; David R. Engelke, Ed., RNA Interference (RNAi) Nuts & Bolts of RNAi Technology, DNA Press, Eagleville, P A (2003); and Gregory J. Hannon, Ed., RNAi A Guide to Gene Silencing, Cold Spring Harbor Laboratory Press, Cold Spring Harbor, NY (2003). Soutschek et al. (2004, Nature 432:173-178) describe a chemical modification to siRNAs that aids in intravenous systemic delivery. Optimizing siRNAs involves consideration of overall G/C content, C/T content at the termini, Tm and the nucleotide content of the 3′ overhang. See, for instance, Schwartz et al., 2003, Cell, 115:199-208 and Khvorova et al., 2003, Cell 115:209-216. Therefore, the present invention also includes methods of decreasing levels of host protein at the protein level using RNAi technology.

In other related aspects, the invention includes an isolated nucleic acid encoding an inhibitor, wherein an inhibitor such as an siRNA or antisense molecule, inhibits the host protein, a derivative thereof, a regulator thereof, or a downstream effector, operably linked to a nucleic acid comprising a promoter/regulatory sequence such that the nucleic acid is preferably capable of directing expression of the protein encoded by the nucleic acid. Thus, the invention encompasses expression vectors and methods for the introduction of exogenous DNA into cells with concomitant expression of the exogenous DNA in the cells such as those described, for example, in Sambrook et al. (2012, Molecular Cloning: A Laboratory Manual, Cold Spring Harbor Laboratory, New York) and as described elsewhere herein. In another aspect of the invention, the host protein or a regulator thereof, can be inhibited by way of inactivating and/or sequestering one or more of the host protein, or a regulator thereof. As such, inhibiting the effects of the host protein can be accomplished by using a transdominant negative mutant.

In another aspect, the invention includes a vector comprising an siRNA or antisense polynucleotide. Preferably, the siRNA or antisense polynucleotide is capable of inhibiting the expression of host protein. The incorporation of a desired polynucleotide into a vector and the choice of vectors is well-known in the art as described in, for example, Sambrook et al., supra.

The siRNA or antisense polynucleotide can be cloned into a number of types of vectors as described elsewhere herein. For expression of the siRNA or antisense polynucleotide, at least one module in each promoter functions to position the start site for RNA synthesis.

In order to assess the expression of the siRNA or antisense polynucleotide, the expression vector to be introduced into a cell can also contain either a selectable marker gene or a reporter gene or both to facilitate identification and selection of expressing cells from the population of cells sought to be transfected or infected through viral vectors. In other embodiments, the selectable marker may be carried on a separate piece of DNA and used in a co-transfection procedure. Both selectable markers and reporter genes may be flanked with appropriate regulatory sequences to enable expression in the host cells. Useful selectable markers are known in the art and include, for example, antibiotic-resistance genes, such as neomycin resistance and the like.

In one embodiment of the invention, an antisense nucleic acid sequence which is expressed by a plasmid vector is used to inhibit the host protein. The antisense expressing vector is used to transfect a mammalian cell or the mammal itself, thereby causing reduced endogenous expression of host protein.

Antisense molecules and their use for inhibiting gene expression are well known in the art (see, e.g., Cohen, 1989, In: Oligodeoxyribonucleotides, Antisense Inhibitors of Gene Expression, CRC Press). Antisense nucleic acids are DNA or RNA molecules that are complementary, as that term is defined elsewhere herein, to at least a portion of a specific mRNA molecule (Weintraub, 1990, Scientific American 262:40). In the cell, antisense nucleic acids hybridize to the corresponding mRNA, forming a double-stranded molecule thereby inhibiting the translation of genes.

The use of antisense methods to inhibit the translation of genes is known in the art, and is described, for example, in Marcus-Sakura (1988, Anal. Biochem. 172:289). Such antisense molecules may be provided to the cell via genetic expression using DNA encoding the antisense molecule as taught by Inoue, 1993, U.S. Pat. No. 5,190,931.

Alternatively, antisense molecules of the invention may be made synthetically and then provided to the cell. In some embodiments, the antisense oligomers are about 10 to about 30, since they are easily synthesized and introduced into a target cell. Synthetic antisense molecules contemplated by the invention include oligonucleotide derivatives known in the art which have improved biological activity compared to unmodified oligonucleotides (see U.S. Pat. No. 5,023,243).

Ribozymes and their use for inhibiting gene expression are also well known in the art (see, e.g., Cech et al., 1992, J. Biol. Chem. 267:17479-17482; Hampel et al., 1989, Biochemistry 28:4929-4933; Eckstein et al., International Publication No. WO 92/07065; Altman et al., U.S. Pat. No. 5,168,053). Ribozymes are RNA molecules possessing the ability to specifically cleave other single-stranded RNA in a manner analogous to DNA restriction endonucleases. Through the modification of nucleotide sequences encoding these RNAs, molecules can be engineered to recognize specific nucleotide sequences in an RNA molecule and cleave it (Cech, 1988, J. Amer. Med. Assn. 260:3030). A major advantage of this approach is the fact that ribozymes are sequence-specific.

There are two basic types of ribozymes, namely, tetrahymena-type (Hasselhoff, 1988, Nature 334:585) and hammerhead-type. Tetrahymena-type ribozymes recognize sequences which are four bases in length, while hammerhead-type ribozymes recognize base sequences 11-18 bases in length. The longer the sequence, the greater the likelihood that the sequence will occur exclusively in the target mRNA species. Consequently, hammerhead-type ribozymes are preferable to tetrahymena-type ribozymes for inactivating specific mRNA species, and 18-base recognition sequences are preferable to shorter recognition sequences which may occur randomly within various unrelated mRNA molecules.

In one embodiment of the invention, a ribozyme is used to inhibit the host protein. Ribozymes useful for inhibiting the expression of a target molecule may be designed by incorporating target sequences into the basic ribozyme structure which are complementary, for example, to the mRNA sequence of the host protein of the present invention. Ribozymes targeting the host protein may be synthesized using commercially available reagents (Applied Biosystems, Inc., Foster City, CA) or they may be genetically expressed from DNA encoding them.

When the inhibitor of the invention is a small molecule, a small molecule antagonist may be obtained using standard methods known to the skilled artisan. Such methods include chemical organic synthesis or biological means. Biological means include purification from a biological source, recombinant synthesis and in vitro translation systems, using methods well known in the art.

Combinatorial libraries of molecularly diverse chemical compounds potentially useful in treating a variety of diseases and conditions are well known in the art as are method of making the libraries. The method may use a variety of techniques well-known to the skilled artisan including solid phase synthesis, solution methods, parallel synthesis of single compounds, synthesis of chemical mixtures, rigid core structures, flexible linear sequences, deconvolution strategies, tagging techniques, and generating unbiased molecular landscapes for lead discovery vs. biased structures for lead development.

In a general method for small library synthesis, an activated core molecule is condensed with a number of building blocks, resulting in a combinatorial library of covalently linked, core-building block ensembles. The shape and rigidity of the core determines the orientation of the building blocks in shape space. The libraries can be biased by changing the core, linkage, or building blocks to target a characterized biological structure (“focused libraries”) or synthesized with less structural bias using flexible cores.

In another aspect of the invention, the host protein can be inhibited by way of inactivating and/or sequestering the host protein. As such, inhibiting the effects of host protein can be accomplished by using a transdominant negative mutant.

In some embodiments, an antibody specific for the host protein (e.g., an antagonist to host protein) may be used. In one embodiment, the antagonist is a protein and/or compound having the desirable property of interacting with a binding partner of host protein (e.g., a microbial cell) and thereby competing with the corresponding protein. In another embodiment, the antagonist is a protein and/or compound having the desirable property of interacting with the host protein and thereby sequestering the host protein.

As will be understood by one skilled in the art, any antibody that can recognize and bind to an antigen of interest is useful in the present invention. Methods of making and using antibodies are well known in the art. For example, polyclonal antibodies useful in the present invention are generated by immunizing rabbits according to standard immunological techniques well-known in the art (see, e.g., Harlow et al., 1988, In: Antibodies, A Laboratory Manual, Cold Spring Harbor, NY). Such techniques include immunizing an animal with a chimeric protein comprising a portion of another protein such as a maltose binding protein or glutathione (GSH) tag polypeptide portion, and/or a moiety such that the antigenic protein of interest is rendered immunogenic (e.g., an antigen of interest conjugated with keyhole limpet hemocyanin, KLH) and a portion comprising the respective antigenic protein amino acid residues. The chimeric proteins are produced by cloning the appropriate nucleic acids encoding the marker protein into a plasmid vector suitable for this purpose, such as but not limited to, pMAL-2 or pCMX.

However, the invention should not be construed as being limited solely to methods and compositions including these antibodies or to these portions of the antigens. Rather, the invention should be construed to include other antibodies, as that term is defined elsewhere herein, to antigens, or portions thereof. Further, the present invention should be construed to encompass antibodies, inter alia, bind to the specific antigens of interest, and they are able to bind the antigen present on Western blots, in solution in enzyme linked immunoassays, in fluorescence activated cells sorting (FACS) assays, in magnetic affinity cell sorting (MACS) assays, and in immunofluorescence microscopy of a cell transiently transfected with a nucleic acid encoding at least a portion of the antigenic protein, for example.

One skilled in the art would appreciate, based upon the disclosure provided herein, that the antibody can specifically bind with any portion of the antigen and the full-length protein can be used to generate antibodies specific therefor. However, the present invention is not limited to using the full-length protein as an immunogen. Rather, the present invention includes using an immunogenic portion of the protein to produce an antibody that specifically binds with a specific antigen. That is, the invention includes immunizing an animal using an immunogenic portion, or antigenic determinant, of the antigen.

Once armed with the sequence of a specific antigen of interest and the detailed analysis localizing the various conserved and non-conserved domains of the protein, the skilled artisan would understand, based upon the disclosure provided herein, how to obtain antibodies specific for the various portions of the antigen using methods well-known in the art or to be developed.

The skilled artisan would appreciate, based upon the disclosure provided herein, that that present invention includes use of a single antibody recognizing a single antigenic epitope but that the invention is not limited to use of a single antibody. Instead, the invention encompasses use of at least one antibody where the antibodies can be directed to the same or different antigenic protein epitopes.

The generation of polyclonal antibodies is accomplished by inoculating the desired animal with the antigen and isolating antibodies which specifically bind the antigen therefrom using standard antibody production methods such as those described in, for example, Harlow et al. (1988, In: Antibodies, A Laboratory Manual, Cold Spring Harbor, NY).

Monoclonal antibodies directed against full length or peptide fragments of a protein or peptide may be prepared using any well-known monoclonal antibody preparation procedures, such as those described, for example, in Harlow et al. (1988, In: Antibodies, A Laboratory Manual, Cold Spring Harbor, NY) and in Tuszynski et al. (1988, Blood, 72:109-115). Quantities of the desired peptide may also be synthesized using chemical synthesis technology. Alternatively, DNA encoding the desired peptide may be cloned and expressed from an appropriate promoter sequence in cells suitable for the generation of large quantities of peptide. Monoclonal antibodies directed against the peptide are generated from mice immunized with the peptide using standard procedures as referenced herein.

Nucleic acid encoding the monoclonal antibody obtained using the procedures described herein may be cloned and sequenced using technology which is available in the art, and is described, for example, in Wright et al. (1992, Critical Rev. Immunol. 12:125-168), and the references cited therein.

Further, the antibody of the invention may be “humanized” using the technology described in, for example, Wright et al., and in the references cited therein, and in Gu et al. (1997, Thrombosis and Hematocyst 77:755-759), and other methods of humanizing antibodies well-known in the art or to be developed.

The present invention also includes the use of humanized antibodies specifically reactive with epitopes of an antigen of interest. The humanized antibodies of the invention have a human framework and have one or more complementarity determining regions (CDRs) from an antibody, typically a mouse antibody, specifically reactive with an antigen of interest. When the antibody used in the invention is humanized, the antibody may be generated as described in Queen, et al. (U.S. Pat. No. 6,180,370), Wright et al., (supra) and in the references cited therein, or in Gu et al. (1997, Thrombosis and Hematocyst 77(4):755-759). The method disclosed in Queen et al. is directed in part toward designing humanized immunoglobulins that are produced by expressing recombinant DNA segments encoding the heavy and light chain complementarity determining regions (CDRs) from a donor immunoglobulin capable of binding to a desired antigen, such as an epitope on an antigen of interest, attached to DNA segments encoding acceptor human framework regions. Generally speaking, the invention in the Queen patent has applicability toward the design of substantially any humanized immunoglobulin. Queen explains that the DNA segments will typically include an expression control DNA sequence operably linked to the humanized immunoglobulin coding sequences, including naturally-associated or heterologous promoter regions. The expression control sequences can be eukaryotic promoter systems in vectors capable of transforming or transfecting eukaryotic host cells or the expression control sequences can be prokaryotic promoter systems in vectors capable of transforming or transfecting prokaryotic host cells. Once the vector has been incorporated into the appropriate host, the host is maintained under conditions suitable for high level expression of the introduced nucleotide sequences and as desired the collection and purification of the humanized light chains, heavy chains, light/heavy chain dimers or intact antibodies, binding fragments or other immunoglobulin forms may follow (Beychok, Cells of Immunoglobulin Synthesis, Academic Press, New York, (1979), which is incorporated herein by reference).

The invention also includes functional equivalents of the antibodies described herein. Functional equivalents have binding characteristics comparable to those of the antibodies, and include, for example, hybridized and single chain antibodies, as well as fragments thereof. Methods of producing such functional equivalents are disclosed in PCT Application WO 93/21319 and PCT Application WO 89/09622.

Functional equivalents include polypeptides with amino acid sequences substantially the same as the amino acid sequence of the variable or hypervariable regions of the antibodies. “Substantially the same” amino acid sequence is defined herein as a sequence with at least 70%, preferably at least about 80%, more preferably at least about 90%, even more preferably at least about 95%, and most preferably at least 99% homology to another amino acid sequence (or any integer in between 70 and 99), as determined by the FASTA search method in accordance with Pearson and Lipman, 1988 Proc. Nat'l. Acad. Sci. USA 85: 2444-2448. Chimeric or other hybrid antibodies have constant regions derived substantially or exclusively from human antibody constant regions and variable regions derived substantially or exclusively from the sequence of the variable region of a monoclonal antibody from each stable hybridoma.

In some embodiments, antibodies include functional antibody fragments for example, a Fab fragment, a single chain Fv fragment, or a heavy chain antibody (such as camelid antibodies). Single chain antibodies (scFv) or Fv fragments are polypeptides that consist of the variable region of the heavy chain of the antibody linked to the variable region of the light chain, with or without an interconnecting linker. Thus, the Fv comprises an antibody combining site.

Functional equivalents of the antibodies of the invention further include fragments of antibodies that have the same, or substantially the same, binding characteristics to those of the whole antibody. Such fragments may contain one or both Fab fragments or the F(ab′)₂ fragment. In some embodiments, the antibody fragments contain all six complement determining regions of the whole antibody, although fragments containing fewer than all of such regions, such as three, four or five complement determining regions, are also functional. For example, in some embodiments, the antibody fragment is a heavy chain antibody (e.g., a nanobody) comprising three complement determining regions. The functional equivalents are members of the IgG immunoglobulin class and subclasses thereof, but may be or may combine with any one of the following immunoglobulin classes: IgM, IgA, IgD, or IgE, and subclasses thereof. Heavy chains of various subclasses, such as the IgG subclasses, are responsible for different effector functions and thus, by choosing the desired heavy chain constant region, hybrid antibodies with desired effector function are produced. Exemplary constant regions are gamma 1 (IgG1), gamma 2 (IgG2), gamma 3 (IgG3), and gamma 4 (IgG4). The light chain constant region can be of the kappa or lambda type.

The immunoglobulins of the present invention can be monovalent, divalent or polyvalent. Monovalent immunoglobulins are dimers (HL) formed of a hybrid heavy chain associated through disulfide bridges with a hybrid light chain. Divalent immunoglobulins are tetramers (H2L2) formed of two dimers associated through at least one disulfide bridge.

### Methods of Use

In one exemplary embodiment, the invention provides methods of use of the compositions of the invention to modulate one or more interaction between a host protein and associated microbe. In some embodiments, the invention provides methods of modulating the interaction of a host protein with the associated microbe as listed in Table 1. For example, in some embodiments, the invention provides methods of inhibiting the interaction of Streptococcus with BACE2. In some embodiments, the invention provides methods of inhibiting the interaction of Fusobacterium with immune-modulatory proteins. In some embodiments, the invention provides methods of inhibiting the interaction of Ruminococcus gnavus with one or more proteins expressed on T cells, including CD7 and members of the TNF receptor superfamily including, but not limited to, TMEM149, TNFRSF1B, TNFRSF4, TNFRSF7, TNFRSF9. In one embodiment, the invention provides methods of inhibiting the interaction of Citrobacter and a fibroblast growth factor (FGF) including, but not limited to, FGF1 and FGF7.

In one embodiment, the present invention provides methods for treatment, inhibition, prevention, or reduction of a disease or disorder in a subject in need thereof using modulator of the invention.

In one embodiment, the disease or disorder is associated with a commensal bacterium. Exemplary commensal microbes include, but are not limited to, peptostreptococcus spp., clostridium spp., lactobacillus spp. (Lactobacillus acidophilus, Lactobacillus crispatus, Lactobacillus johnsonii, Lactobacillus sakei, Lactobacillus bulgaris, Lactobacillus jensenii, Lactobacillus rhamonsus, Lactobacillus reuteri, Lactobacillus casei var rhamnosus, Lactobacillus gasseri, Lactobacillus fermentum, Lactobacillus iners, Lactobacillus helveticus, Lactobacillus leichmannii, Lactobacillus brevis, Lactobacillus plantarum, Lactobacillus delbrueckii, Lactobacillus vaginalis, Lactobacillus salivarius, Lactobacillus coleohominis, Lactobacillus pentosus, propionibacerium spp., eubacterium spp., bifidobacterium spp., prevotella spp., bacteroides spp., fusobacterium spp., veillonella spp., diphtheroides spp., and actinomycetales spp.

In one embodiment, the disease or disorder is associated with at least one potentially pathogenic commensal microbe (i.e., pathobiont). Exemplary pathobionts include, but are not limited to, helicobacter spp., segmented filamentous bacteria, pathogenic Bacteroides fragilis strains, pathogenic Enterobacter spp., pathogenic Prevotellaceae spp., pathogenic Erysipelotrichaceae spp., Klebsiella spp. and pathogenic Clostridia spp.

In one embodiment, the disease or disorder is associated with a strain of Erysipelotrichaceae or Proteus mirabilis such as, but not limited to, Erysiptelotrichaceae strain NWP_0324 (“Ery128”), Proteus mirabilis strain WGLW6 (NWP59).

Examples of diseases or disorders that can be treated or prevented by modulating host-microbiome interactions include, but are not limited to inflammatory diseases, inflammatory bowel disease (IBD), irritable bowel syndrome (IBS), obesity, type 2 diabetes, cardiovascular disease, Alzheimer's disease, Parkinson's disease, cancer and atopy.

In one exemplary embodiment, the method includes treating or preventing Alzheimer's disease through inhibiting the interaction of Streptococcus with BACE2. In one exemplary embodiment, the method includes treating or preventing cancer through inhibiting the interaction of Fusobacterium with immune-modulatory proteins including, but not limited to, AGER, BTN3A3, BTNL8, C3AR, CCL5, CCR9, CD55, CD99L2, CEACAM4, CSF3, DKK1, DKK2, IL15RA, LAIR1, MADCAM1, MCAM, MERTK, NPY5R, SIRPA, SOST, TMEM149, TNFRSF10B, TNFRSF4, and TREML1. In some embodiments, the cancer is colon cancer.

In one exemplary embodiment, the method includes treating or preventing inflammatory diseases such as IBD through inhibiting the interaction of Ruminococcus gnavus with proteins expressed on T cells, including CD7, TFF1, TFF2, and TFF3. In one embodiment, the method includes methods of treating or preventing inflammatory diseases such as IBD through inhibiting the interaction of RG151 or NWP327 to proteins expressed on T cells, including CD7, TFF1, TFF2, and TFF3.

In one embodiment, the method includes treating or preventing inflammatory diseases through inhibiting the interaction of Citrobacter and a fibroblast growth factor (FGF) including, but not limited to, FGF1 and FGF7.

Therapeutic Compositions

In one embodiment, the invention relates to therapeutic composition comprising a molecule, such as a protein or peptide, that modulates a host-microbe interaction. Such a molecule (e.g., protein or peptide, etc.) and the encoding nucleic acid sequence may then serve as a target for modulating host-microbe interaction in the host organism. In one embodiment, the molecule inhibits host-microbe interactions. In one embodiment, the molecule promotes host-microbe interactions. In one embodiment, the therapeutic modulates interactions between host proteins and microbes identified using the methods according to the present invention. In one embodiment, the therapeutic modulates interactions between host proteins and microbes identified in Table 1.

In various embodiments, the invention relates to compositions comprising therapeutic agents identified using the methods described herein and their use in treating or preventing a disease or disorder associated with a microbe. In one embodiment, the invention relates to compositions comprising therapeutic agents and their use in treating or preventing a disease or disorder associated with one or more microbes as set forth in Table 1.

In one embodiment, the method of identifying a therapeutic agent that modulates a host:microbe interaction comprises performing an appropriate assay in the presence of one or more candidate agent and evaluating the effect of the agent on the ability of at least one microbe to interact with its associated host protein as set forth in Table 1. In one embodiment, the therapeutic agent is one that increases or promotes interaction of a microbe with its associated host protein. In one embodiment, the interaction is increased by at least 2%, 3%, 4%, 5%, 6%, 7%, 8%, 9%, 10%, 20%, 30%, 40%, 50%, 75%, 100%, or greater than 100% compared to a control value. In one embodiment, the therapeutic agent is one that decreases or inhibits interaction of a microbe with its associated host protein. In one embodiment, the interaction is decreased or inhibited by at least 2%, 3%, 4%, 5%, 6%, 7%, 8%, 9%, 10%, 20%, 30%, 40%, 50%, 75%, 100%, or greater than 100% compared to a control value.

In one embodiment, the present invention relates to a composition comprising a therapeutic agent that interacts with a microbe to modulate its interaction with its associated host protein. In one embodiment, the present invention relates to a composition comprising a therapeutic agent that interacts with a host protein to modulate its interaction with a microbe.

In one embodiment, the composition comprises an antibody or fragment thereof. In some embodiments, the antibody or fragment thereof is useful for therapeutic applications. For example, in some embodiments, the antibody or fragment thereof, can be used to reduce or eliminate pathogenic or potentially pathogenic commensal bacteria, or inhibit bacterial pathogenicity. In some embodiments, the antibody or fragment thereof, is modified to produce precision antibiotics that specifically target a bacteria of interest. For example, in some embodiments, the antibody or fragment thereof is fused to an antibiotic, bacteriolysin, bacteriocin, or other compound that results in the reduction of the pathogenicity of a pathogenic or potentially pathogenic commensal bacterium. For example, in one embodiment, the composition comprises an antibody or fragment thereof fused, linked, or otherwise attached to a delivery vehicle comprising an antibiotic or other agent that reduces the pathogenicity of a pathogenic or potentially pathogenic commensal bacterium. In some embodiments, the antibody or fragment thereof is fused to an agent that promotes the growth of beneficial bacteria. For example, the antibody or fragment thereof can be fused to specific growth-promoting nutrients. In one embodiment, the composition comprises an antibody or fragment thereof fused, linked, or otherwise attached to a delivery vehicle comprising a growth-promoting agent.

In one embodiment, the therapeutic agent comprises a bispecific antibody targeting both a microbe and a display molecule. Exemplary bispecific antibodies include antibodies targeting a microbe and further targeting a display molecule comprising an antibody, antibody fragment or antibody mimetic. In one embodiment, a bispecific antibody targets an IgG, IgA, IgM, or IgE antibody. Such an embodiment is useful, for example, in targeting antibodies to a microbe of interest.

In one embodiment, the bispecific antibody comprises a region that binds to a specific microbe of Table 1 to inhibit the interaction of the microbe with the associated host protein. In one embodiment, the bispecific antibody comprises a region that binds to a specific host protein of Table 1 to inhibit the interaction of the host protein with the associated microbe. In one embodiment, the bispecific antibody further comprises a region that binds to luminal IgA.

In some embodiments, the composition comprises a therapeutic or diagnostic agent comprising an affinity reagent described herein, including, but not limited to nanobodies, conventional antibodies, affibodies, anticalins, and monobodies. In some embodiments, the therapeutic or diagnostic agent comprises an antibody or fragment thereof, that specifically binds a to a specific microbe of Table 1 to detect or inhibit the interaction of the microbe with the associated host protein. In one embodiment, the therapeutic or diagnostic agent comprises an antibody or fragment thereof, that binds to a specific host protein of Table 1 to detect or inhibit the interaction of the host protein with the associated microbe.

In one embodiment, the invention relates to methods of treatment or prevention of a disease or disorder associated with a microbe using the therapeutic agents of the invention. In one embodiment, the invention relates to methods of treatment or prevention of a disease or disorder associated with at least one host-microbe interaction identified in Table 1 using the therapeutic agents of the invention. Administration of the therapeutic agent in accordance with the present invention may be continuous or intermittent, depending, for example, upon the recipient's physiological condition, whether the purpose of the administration is therapeutic or prophylactic, and other factors known to skilled practitioners. The administration of the agents of the invention may be essentially continuous over a preselected period of time or may be in a series of spaced doses. Both local and systemic administration is contemplated. The amount administered will vary depending on various factors including, but not limited to, the composition chosen, the particular disease, the weight, the physical condition, and the age of the subject, and whether prevention or treatment is to be achieved. Such factors can be readily determined by the clinician employing animal models or other test systems which are well known to the art

Kits

The present invention also pertains to kits useful in the methods of the invention. Such kits comprise various combinations of components useful in any of the methods described elsewhere herein. For example, in one embodiment, the kit comprises components useful for modulating one or more host protein-microbial cell interaction as described herein. In one embodiment, the kit contains additional components. In one embodiment, an additional component includes but is not limited to instructional material. In one embodiment, instructional material for use with a kit of the invention may be provided electronically.

### Experimental Examples

The invention is further described in detail by reference to the following experimental examples. These examples are provided for purposes of illustration only, and are not intended to be limiting unless otherwise specified. Thus, the invention should in no way be construed as being limited to the following examples, but rather, should be construed to encompass any and all variations which become evident as a result of the teaching provided herein.

Without further description, it is believed that one of ordinary skill in the art can, using the preceding description and the following illustrative examples, make and utilize the present invention and practice the claimed methods. The following working examples therefore are not to be construed as limiting in any way the remainder of the disclosure.

### Example 1: A Tool to Discover Bacterial Interactions with the Host Exoproteome

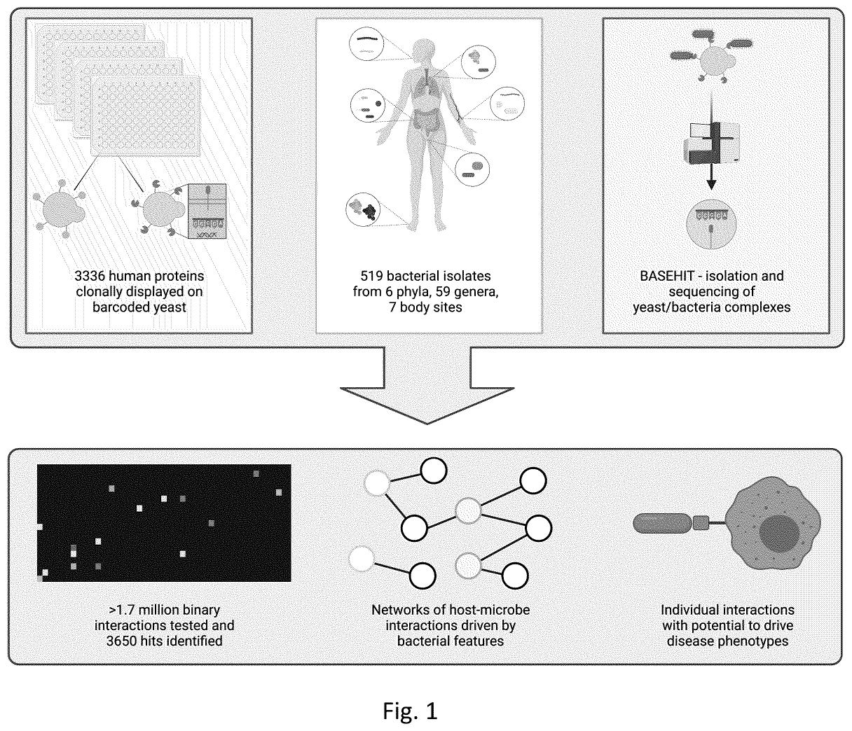

A high-throughput technology to screen intact microbial cells for the ability to bind to human proteins displayed on the surface of yeast was developed, optimized, and deemed BASEHIT (FIG. 1). The BASEHIT process involves light biotinylation of the bacterial cell surface, mixing of bacterial cells with barcoded yeast clones that display individual human extracellular proteins, isolation of bacterially-bound yeast, and next-generation sequencing of the barcodes encoded by the enriched yeast clones (FIG. 1). Using these data, a “BASEHIT Score” is derived that represents the predicted degree of interaction between an individual microbe and a given host protein.

To enable simultaneous evaluation of potential bacterial binding to thousands of human proteins, a barcoded library of 3336 human extracellular and secreted proteins (the “exoproteome”) displayed clonally on the surface of yeast was created and validated. This library covers over 50% of the total human exoproteome and includes a broad range of transmembrane, secreted, and membrane-associated proteins with diverse protein folds, expression patterns, and biological functions.

A Draft Atlas of the Host-Microbiota Interactome

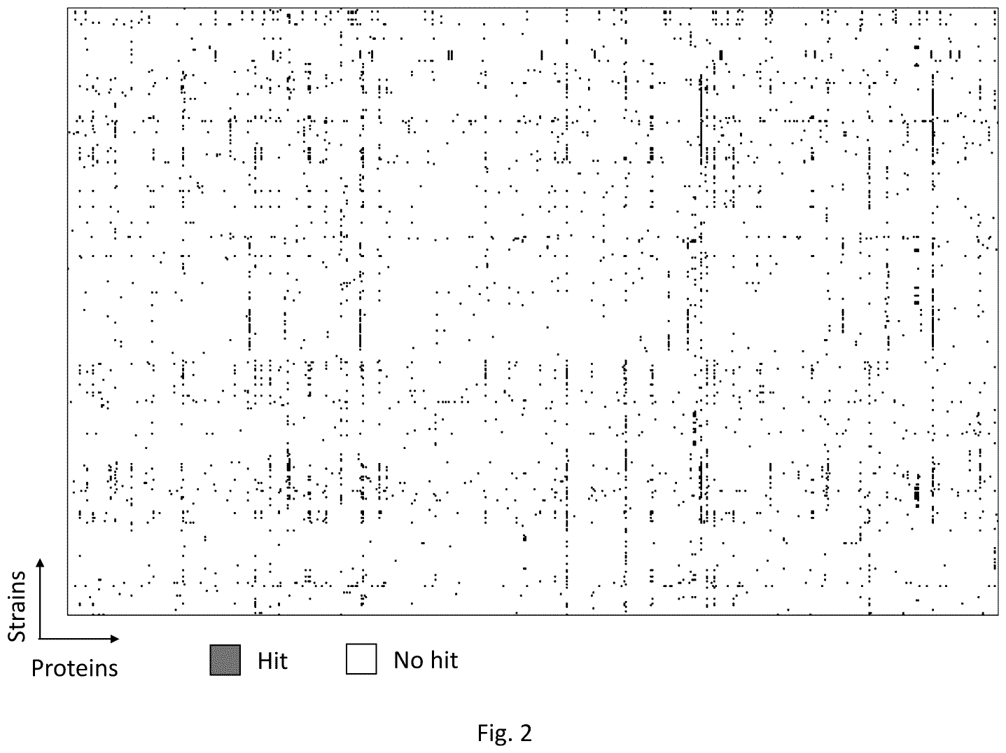

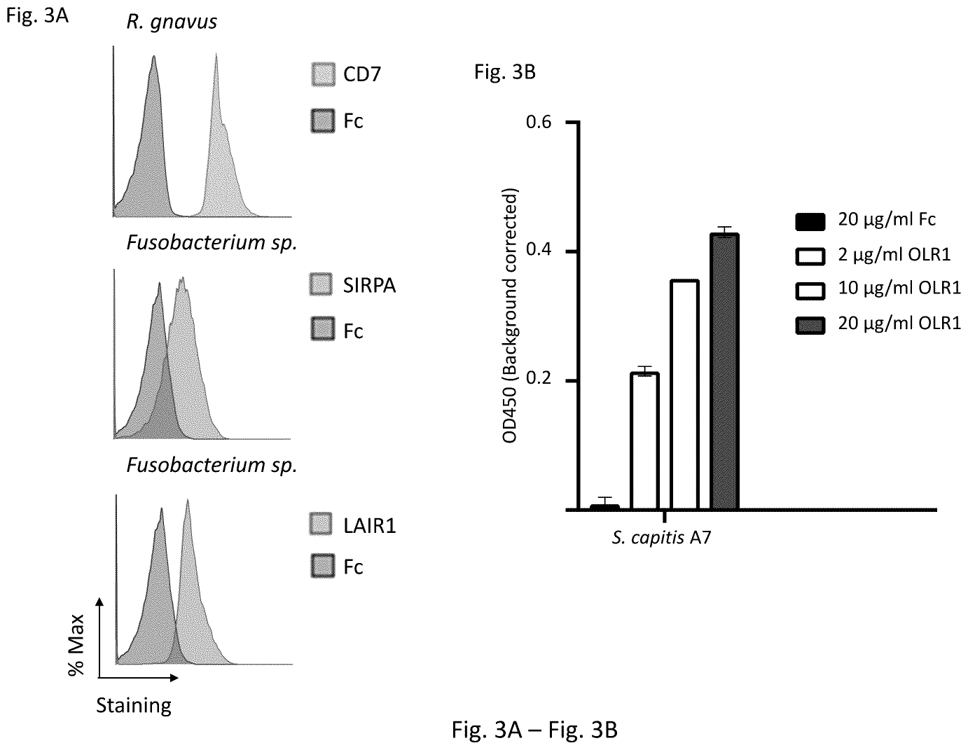

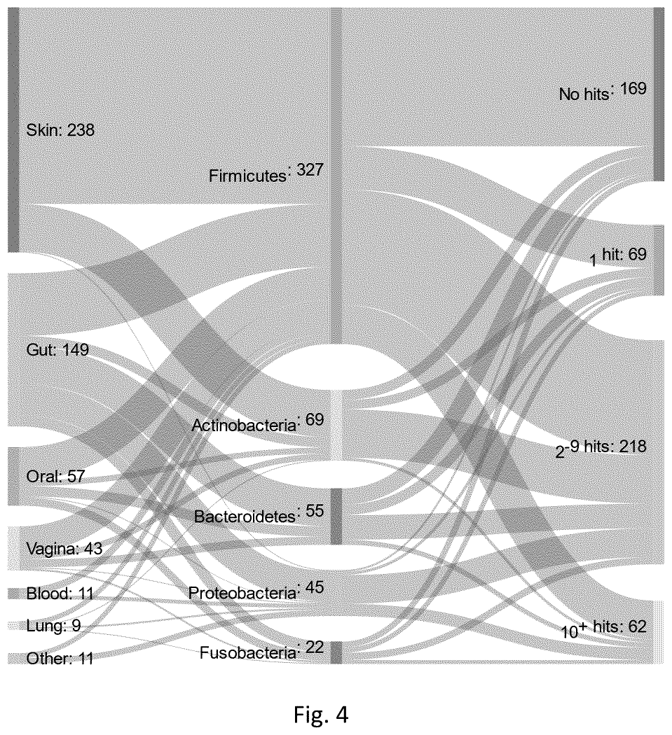

Next, BASEHIT was used to begin to map the landscape of host-microbiota interactions across diverse bacterial phylogenies and tissue sites. A collection of 519 bacterial strains isolated from the mouth, gut, lung, skin, or vagina that spanned 6 phyla and 59 genera was assembled and screened. This collection also included multiple strains from numerous species to evaluate the impact of strain variation on host-microbiome interactions. In total, over 1.7 million potential binary interactions between individual host proteins and unique bacterial strains were interrogated and 3650 predicted binding events that passed the BASEHIT score cutoff were identified (FIG. 2). To test the veracity of the BASEHIT results, selected interactions were validated via either ELISA or flow cytometry, which demonstrates that BASEHIT has high predictive value for identifying novel host-microbe interactions (FIG. 3A-B). This screen therefore revealed thousands of newly predicted molecular-level host-microbiome interactions and dramatically expands the collective understanding of the mechanisms by which indigenous microbes can potentially interface with and impact the host.

The thousands of predicted interactions that were identified span diverse phylogenies, tissues, and proteins, and thus begin to define the distributions of interactions across bacterial taxonomies and tissues of origin, as well as host protein families and folds (FIG. 4). With a few notable exceptions, the distribution of host interactions was highly variable across isolates both within and between phylogenetic groups, and across different tissues of origin. Thus, whether and how members of the microbiome directly interface with host proteins is unpredictable based on their phylogeny or tissue of origin.

Interaction patterns also varied widely across protein families and folds. For example, 2705 (81%) of the 3336 proteins in the present library failed to interact significantly with any bacterial strains in the present culture collections, 261 proteins interacted with only one strain, and only 42 (1.2%) proteins interacted with 10 or more strains. Lack of predicted interactions was not due to failure of protein expression or aberrant protein folding in the yeast library as many non-interacting proteins were readily detected via staining with specific antibodies. Proteins that bound to multiple taxa spanned diverse protein families and folds.

Divergent Host Protein Binding Patterns Imply Functional Variation Between Phylogenetically-Related Bacterial Strains

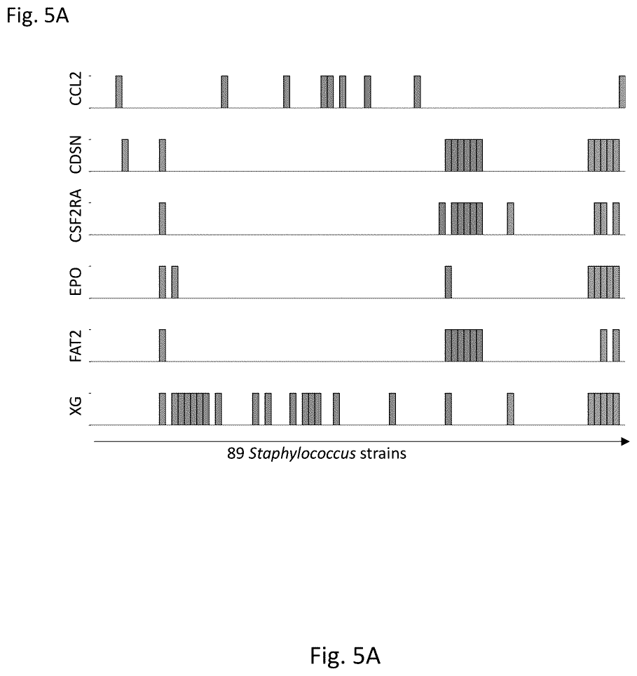

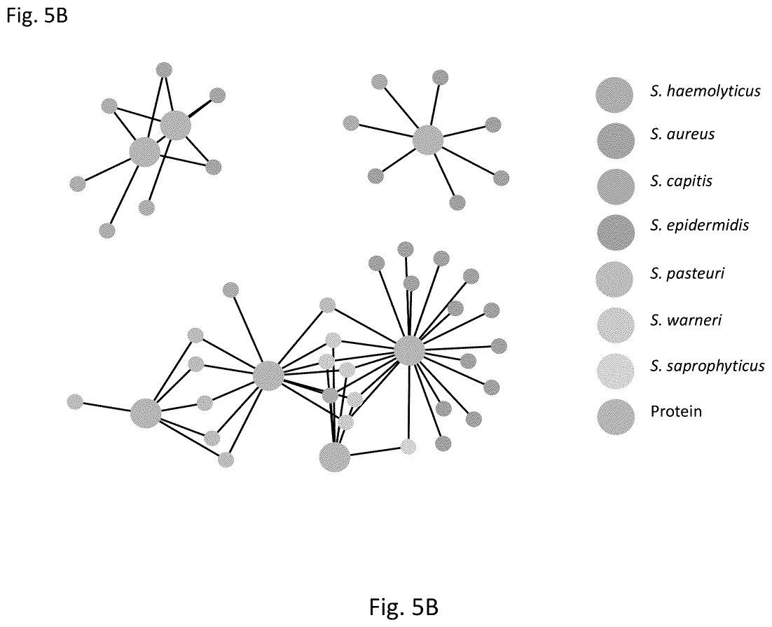

The present collections of this Example included a deep sampling of skin-derived Staphylococcus isolates from five common Staphylococcus species, so patterns of host-microbiota interaction were compared across dozens of bacterial strains assigned to the same species (FIG. 5A). Multiple Staphylococcus isolates interacted with known skin-expressed proteins, including the corneodesmosome protein CDSN, junctional protein FAT2, and hematopoietic or epithelial related proteins CCL2, CSF2RA, EPO, and XG. However, protein-binding patterns varied dramatically both within and between species, and most interactions were highly strain-specific even within a given species designation. Strain variation can also be made clear by visualizing the network of interactions between strains and host proteins, where multiple distinct sub-clusters are visible that can separate related strains (FIG. 5B). These results demonstrate that patterns of host interaction vary dramatically among strains from the same species that occupy similar host-niches and imply functional variation between phylogenetically-related bacterial strains.

Disease-Relevant Gut Commensals Exhibit Unique Host Protein-Binding Patterns

Next, two bacterial taxa associated with human disease, the genus Fusobacterium and the species Ruminococcus gnavus, were interrogated.

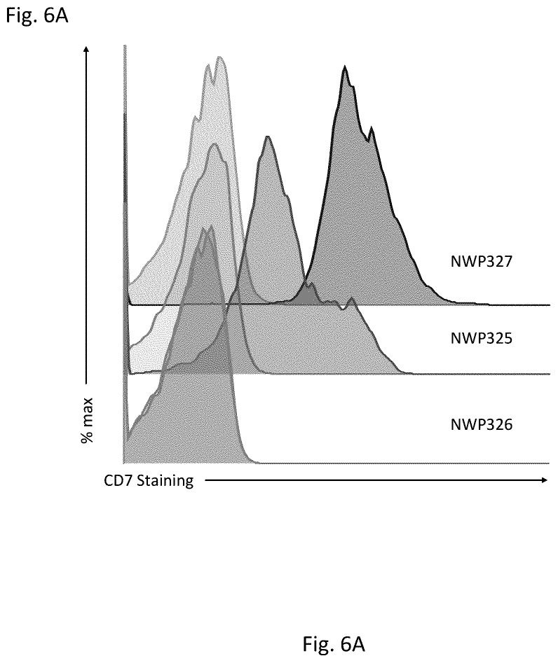

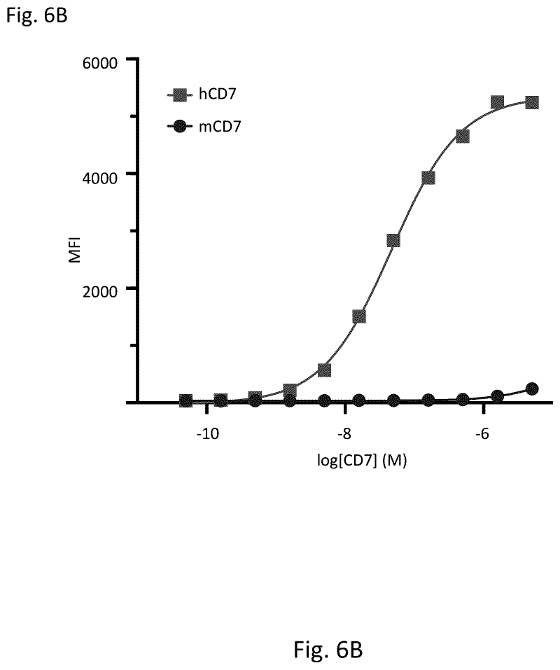

R. gnavus is enriched in inflammatory bowel disease (IBD) subjects, and 20 strain-level variation and “clade switching” correlates with IBD flares. Two R. gnavus isolates exhibited strong interactions with the T cell protein CD7. Additionally, R. gnavus has been shown to exhibit high aerotolerance and mucosal localization, which is additionally supported by the strong interaction with the mucus-associated trefoil factors TFF1, TFF2, and TFF3. These interactions may provide direct molecular insight into the mechanism of R. gnavus modulation of human inflammatory disease. Close examination of CD7 binding by R. gnavus revealed specificity towards both bacterial strains and host species. Bacterial flow cytometry of three distinct isolates, all from IBD patients, showed that two strains bound CD7 (at variable levels), while one strain showed no CD7 binding activity (FIG. 6A). Similarly, the host species specificity of the R. gnavus-CD7 interaction was examined by staining with recombinant mouse or human CD7, and no binding of mouse CD7, even to the strain (NWP327) with the highest CD7 binding activity, was observed (FIG. 6B). These results emphasize the potentially complex nature and co-evolution of host-microbe interactions, and highlight the need for careful consideration of precise molecular function in strains to unravel associations with human disease.

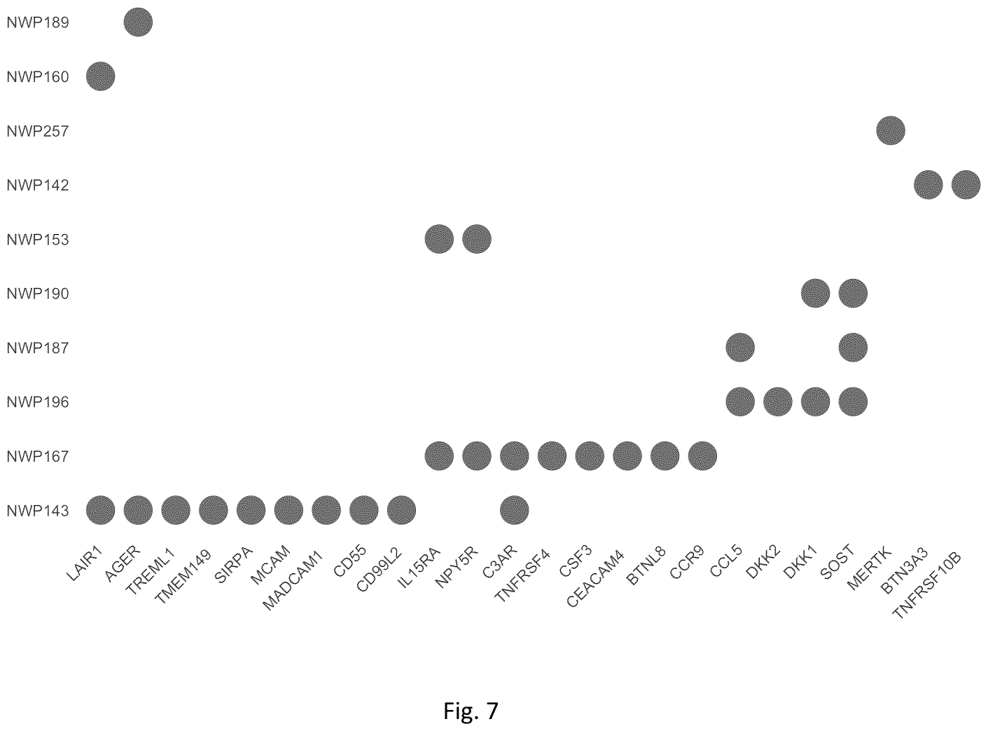

Fusobacterium species, particularly Fusobacterium nucleatum, are enriched in tumors and modulate host immunity through direct interactions. The seventeen Fusobacterium strains in the present collection spanned five tissues of origin. However, many Fusobacterium isolates bound to at least one known immunomodulatory host protein or key epithelial proteins potentially involved in tumorigenesis (FIG. 7). These interactions include two ITIM-coupled immunosuppressive proteins (e.g., SIRPA and LAIR1), cytokine receptors (e.g., IL15RA, TNFRSF4, and TNFRSF10B), cytokines and chemokines (CSF3, CCL5), WNT-pathway modulators (DKK1, DKK2, SOST), and adhesion proteins (CEACAM4, MCAM, MADCAM1). The breadth of cell types and immunological processes targeted by Fusobacterium strains build upon the previously identified strain-specific interaction with the immunoreceptors TIGIT and CEACAM1 and expand the range of Fusobacterium immunomodulatory potential. Mounting evidence demonstrates that the direct interaction of F. nucleatum strains with immune and tumor cells directly modulates important steps of metastasis and tumor killing, and these newly discovered interactions may provide molecular insight into these effects.

The High-Throughput Screening Tool, BASEHIT, Reveals Novel Host-Microbe Interactions

This Example identifies thousands of direct host-microbe interactions spanning a range of tissues and protein functions, offering new insight into the mechanisms by which microbes may influence host physiology and colonize host-associated niches. These interactions may underlie the ability of microbes to enter non-mucosal, supposedly “sterile”, tissues, and exert effects on the host upon entering those tissues. In particular, these data complement recent observations of tumor-associated microbiomes that influence treatment efficacy, as mechanisms for immune modulation or tissue colonization may enable selective translocation, survival, and activity of particular microbial strains into the tumor microenvironment.

Importantly, BASEHIT enables a phylogenetically unbiased description of microbial function. While some interactions were phylogenetically biased, substantial variation in individual interactions among closely related strains and shared interactions across diverse microbes were observed, highlighting that direct host interactions are not predictable simply from bacterial phylogeny. Additionally, BASEHIT does not require prior annotation of bacterial genes. As more than 40% of bacterial genes in human-associated metagenomes lack any annotated function, the ability to highlight microbes with direct host associations in a high-throughput manner will complement expanding sequencing-based efforts to define tissue-specific adaptations and effects of commensal microbes.

This Example highlights the complex network of direct host-microbe interactions present within the human microbiota. These interactions may inform the role of microbes in shaping host physiology, and reflect tissue-specific adaptations of microbes to their unique niches. They additionally reveal candidate host pathways that may control or respond to the microbiota. Additionally, a technology that vastly enhances the rate at which new host-microbe interactions may be identified in an unbiased fashion, and which is applicable to a broad range of culturable microbes, has been described herein. This functional-profiling approach should offer further insight into the mechanisms of microbial control of host physiology, the evolution of microbes in specific host-associated niches, and offer new targets for host-directed investigation into microbially mediated disease.

TABLE 1
Paired host protein - microbial interactions

| Strain Name | Strain ID | Protein |
|---|---|---|
| Streptococcus sp., Strain CM6 | NWP136 | SLC10A4 |
| Lachnospiraceae sp., Oral Taxon 082, Strain F0431 | NWP232 | TFF2 |
| Lachnospiraceae sp., Oral Taxon 107, Strain F0167 | NWP262 | TFF2 |
| Lachnospiraceae sp., Oral Taxon 082, Strain F0431 | NWP232 | TFF3 |
| Lachnospiraceae sp., Oral Taxon 107, Strain F0167 | NWP262 | TFF3 |
| Lachnospiraceae sp., Strain ICM7 | NWP241 | TFF3 |
| Escherichia coli, Strain MS 21-1 | NWP38 | CEACAM1 |
| Rothia mucilaginosa, Strain CC87LB | NWP84 | LGALS7 |
| Lactobacillus crispatus, Strain GED7749C | NWP104 | CGA |
| Staphylococcus warneri, Strain SK66 | NWP11 | PSORS1C2 |
| Bacteroides dorei, Strain 5_1_36/D4 | NWP244 | EPHA2 |
| Finegoldia magna, Strain SY01 | NWP138 | XG |
| Lachnospiraceae sp., Strain ICM7 | NWP241 | TFF2 |
| Lactobacillus gasseri, Strain JV-V03 | NWP106 | TFF3 |
| Ruminococcus gnavus, Strain NWP327 | RG151 | TFF2 |
| Lactobacillus jensenii, Strain 208-1 | NWP111 | TFF3 |
| Lachnospiraceae sp., Oral Taxon 107, Strain F0167 | NWP262 | TFF1 |
| Lactobacillus jensenii, Strain 208-1 | NWP111 | TFF2 |
| Lactobacillus crispatus, Strain GED7749C | NWP104 | STIM2 |
| Lactobacillus gasseri, Strain JV-V03 | NWP106 | TFF2 |
| Bacteroidales bacterium, Strain KA00344 | NWP302 | LCN2 |
| Staphylococcus warneri, Strain SK66 | NWP11 | PPY |
| Staphylococcus pasteuri C9 | pasteuriC9 | GPR15L |
| Lachnospiraceae sp., Strain ICM7 | NWP241 | TFF1 |
| Lachnospiraceae sp., Oral Taxon 082, Strain F0431 | NWP232 | TFF1 |
| Staphylococcus capitis B12 | capitisB12 | PSORS1C2 |
| Staphylococcus pasteuri C9 | pasteuriC9 | XG |
| Ruminococcus gnavus, Strain NWP327 | RG151 | TFF3 |
| Bacteroidales bacterium, Strain KA00344 | NWP302 | MYPR_Epitope-2 |
| Escherichia coli, Strain MS 21-1 | NWP38 | CD55 |
| Bacteroides dorei, Strain 5_1_36/D4 | NWP244 | MCAM |
| Bacteroides sp., Strain 4_1_36 | NWP312 | SMR3A |
| Staphylococcus epidermidis G4 | EpiG4 | XG |
| Staphylococcus warneri, Strain SK66 | NWP11 | FGFR1 |
| Lactobacillus rhamnosus, Strain LMS2-1 | NWP97 | TFF2 |
| Lactobacillus crispatus, Strain JV-V01 | NWP107 | TFF2 |
| Staphylococcus warneri, Strain SK66 | NWP11 | XG |
| Lactobacillus parafarraginis, Strain F0439 | NWP105 | TFF2 |
| Oscillibacter sp., Strain KLE-1728 | NWP269 | PDIA6 |
| Prevotella disiens, Strain DNF00882 | NWP177 | CLEC4M |
| Lactobacillus crispatus, Strain JV-V01 | NWP107 | TFF3 |
| Bacteroides fragilis, Strain CL05T00C42 | NWP287 | OTOR |
| Lactobacillus crispatus, Strain GED7749C | NWP104 | CLU |
| Ruminococcus gnavus, Strain NWP327 | RG151 | TFF1 |
| Bacteroidales bacterium, Strain KA00344 | NWP302 | PDGFA |
| Lactobacillus jensenii, Strain EX849587VC03 | NWP108 | TFF2 |
| Staphylococcus warneri E6 | warneriE6 | GPR15L |
| Staphylococcus warneri, Strain SK66 | NWP11 | CSPG5 |
| Staphylococcus warneri E6 | warneriE6 | XG |
| Lactobacillus jensenii, Strain 269-3 | NWP110 | TFF3 |
| Staphylococcus warneri D11 | warneriD12 | XG |
| Bacteroidales bacterium, Strain KA00344 | NWP302 | PSORS1C2 |
| Lactobacillus parafarraginis, Strain F0439 | NWP105 | TFF3 |
| Staphylococcus warneri D11 | warneriD12 | SLCO2B1 |
| Lachnospiraceae bacterium, Strain AAB19 | AB19 | UNQ9165/PRO28630 |
| Lactobacillus crispatus, Strain GED7749C | NWP104 | ENDOU |
| Staphylococcus warneri D11 | warneriD12 | GPR15L |
| Capnocytophaga sputigena, Strain CC21_001D | NWP163 | CST4 |
| Staphylococcus warneri E6 | warneriE6 | SLCO2B1 |
| Bacteroides dorei, Strain 5_1_36/D4 | NWP244 | EPHA5 |
| Proteus mirabilis, Strain WGLW6 | NWP59 | NGFR |
| Staphylococcus pasteuri C9 | pasteuriC9 | EPO |
| Staphylococcus capitis B12 | capitisB12 | F3 |
| Clostridium orbiscindens, Strain CC43_001K | NWP259 | PGLYRP1 |
| Lactobacillus jensenii, Strain EX849587VC03 | NWP108 | TFF3 |
| Staphylococcus warneri D11 | warneriD12 | RLN2 |
| Staphylococcus warneri E6 | warneriE6 | RLN2 |
| Fusobacterium sp., Strain CM22 | NWP153 | IL15RA |
| Klebsiella oxytoca, Strain MIT 10-5249 | NWP52 | SLC22A5 |
| Streptococcus parasanguinis, Strain F0449 | NWP114 | GCGR |
| Staphylococcus warneri E4 | warneriE4 | XG |
| Staphylococcus warneri E4 | warneriE4 | GPR15L |
| Staphylococcus warneri, Strain SK66 | NWP11 | CXCR3 |
| Bacteroidales bacterium, Strain KA00344 | NWP302 | RCN1 |
| Lactobacillus jensenii, Strain 269-3 | NWP110 | TFF2 |
| Staphylococcus warneri E4 | warneriE4 | SLCO2B1 |
| Staphylococcus pasteuri C9 | pasteuriC9 | MEPE |
| Staphylococcus epidermidis G10 | EpiG10 | PDIA6 |
| Proteus mirabilis, Strain WGLW6 | NWP59 | AXL |
| Prevotella melaninogenica, Strain D18 | NWP151 | PDIA3 |
| Staphylococcus warneri B9 | warneriB9 | PSORS1C2 |
| Staphylococcus warneri E4 | warneriE4 | RLN2 |
| Ruminococcus gnavus, Strain NWP325 | RG74 | TFF2 |
| Streptococcus downei, Oral Taxon 594, Strain F0415 | NWP123 | CTRB1 |
| Staphylococcus pasteuri C9 | pasteuriC9 | GPC1 |
| Streptococcus sp., Strain OBRC6 | NWP124 | CTRB1 |
| Weissella cibaria, Strain F16_1 | NWP101 | TFF2 |
| Prevotella nigrescens, Strain F0103 | NWP145 | T4S19_Epitope-2 |
| Propionibacterium acnes, Strain HL044PA1 | NWP237 | TFF2 |
| Streptococcus sobrinus, Strain W1703 | NWP85 | LOC644613 |
| Lactobacillus jensenii, Strain EX849587VC06 | NWP109 | TFF2 |
| Lactobacillus rhamnosus, Strain LMS2-1 | NWP97 | TFF3 |
| Lachnospiraceae bacterium, Strain AAB26 | AB26 | TMEM119 |
| Sporosarcina sp., Strain 2681 | NWP29 | LOC644613 |
| Achromobacter xylosoxidans, Strain C54 | NWP22 | PF4V1 |
| Fusobacterium nucleatum, Strain CTI-03 | NWP187 | SOST |
| Staphylococcus capitis C11 | capitisC11 | PDIA6 |
| Staphylococcus capitis A7 | capitisA7 | PROCR |
| Propionibacterium acnes, Strain HL002PA3 | NWP219 | TMEM119 |
| Bacteroides fragilis, Strain 3_1_12 | NWP271 | ENDOU |
| Lactobacillus crispatus, Strain GED7749C | NWP104 | GREM2 |
| Staphylococcus hominis, Strain C80 | NWP25 | MUCL3 |
| Fusobacterium sp., Strain CM22 | NWP167 | SGCA |
| Klebsiella oxytoca, Strain MIT 10-5249 | NWP52 | SLC22A4 |
| Streptococcus sp., Strain CM6 | NWP136 | SMR3A |
| Staphylococcus warneri, Strain SK66 | NWP11 | SDC4 |
| Streptococcus downei, Oral Taxon 594, Strain F0415 | NWP123 | RTBDN |
| Prevotella timonensis, Strain CRIS 5C-B1 | NWP156 | PDIA6 |
| Staphylococcus haemolyticus B6 | haemolyticusB6 | SLC8B1 |
| Lactobacillus gasseri, Strain JV-V03 | NWP106 | ENDOU |
| Corynebacterium sp., Strain HFH0082 | NWP61 | LOC644613 |
| Streptococcus downei, Oral Taxon 594, Strain F0415 | NWP123 | LOC644613 |
| Escherichia coli, Strain MS 85-1 | NWP30 | LOC644613 |
| Staphylococcus aureus, Strain MN8 | NWP17 | PF4V1 |
| Clostridium cadaveris, Strain CC40_001C | NWP246 | EPOR |
| Fusobacterium nucleatum subsp. animalis, Oral Taxon 420, Strain F0419 | NWP196 | DRAXIN |
| Staphylococcus pasteuri G3 | pasteuriG3 | PPY |
| Streptococcus sp., Strain CM6 | NWP136 | SMR3B |
| Staphylococcus epidermidis, Strain NIHLM067 | NWP68 | XG |
| Escherichia coli, Strain MS 110-3 | NWP35 | LOC644613 |
| Bacteroides dorei, Strain 5_1_36/D4 | NWP244 | CD248 |
| Lactobacillus parafarraginis, Strain F0439 | NWP105 | MUCL3 |
| Staphylococcus caprae, Strain C87 | NWP24 | MUCL3 |
| Staphylococcus warneri B9 | warneriB9 | SLCO2B1 |
| Enterococcus faecium, Strain E417 | NWP79 | LOC644613 |
| Streptococcus downei, Oral Taxon 594, Strain F0415 | NWP123 | MUCL3 |
| Rothia mucilaginosa, Strain CC87LB | NWP84 | LGALS9 |
| Prevotella bivia, Strain DNF00188 | NWP193 | RCN1 |
| Staphylococcus capitis B12 | capitisB12 | SLC24A3 |
| Staphylococcus warneri E6 | warneriE6 | TFPI2 |
| Enterococcus faecium, Strain TX0133a04 | NWP44 | SYCN |
| Prevotella timonensis, Strain CRIS 5C-B1 | NWP156 | PDIA3 |
| Staphylococcus warneri, Strain SK66 | NWP11 | FGFR2 |
| Staphylococcus capitis A7 | capitisA7 | XG |
| Klebsiella oxytoca, Strain MIT 10-5243 | NWP47 | PF4V1 |
| Staphylococcus pasteuri E5 | pasteuriE5 | PPY |
| Staphylococcus pasteuri F12 | pasteuriF12 | PPY |
| Corynebacterium amycolatum, Strain SK46 | NWP07 | LOC644613 |
| Staphylococcus warneri E6 | warneriE6 | PROCR |
| Enterococcus faecium, Strain TX1330 | NWP19 | LOC644613 |
| Prevotella oralis, Strain HGA0225 | NWP147 | PDIA3 |
| Klebsiella sp., Strain 4_1_44FAA | NWP21 | PF4V1 |
| Lactobacillus parafarraginis, Strain F0439 | NWP105 | LOC644613 |
| Escherichia coli, Strain MS 115-1 | NWP36 | LOC644613 |
| Enterococcus faecium, Strain ERV165 | NWP81 | LOC644613 |
| Prevotella bivia, Strain DNF00650 | NWP176 | MSLN |
| Prevotella disiens, Strain DNF00882 | NWP177 | SHISA7 |
| Dermabacter sp., Strain HFH0086 | NWP92 | CLU |
| Bacteroides sp., Strain AAB9 | AB9 | MCAM |
| Enterococcus faecium, Strain ERV102 | NWP80 | LOC644613 |
| Veillonella sp., Oral Taxon 780, Strain F0422 | NWP268 | PDIA6 |
| Prevotella bivia, Strain DNF00650 | NWP176 | RCN1 |
| Lactobacillus jensenii, Strain 208-1 | NWP111 | LOC644613 |
| Staphylococcus capitis A7 | capitisA7 | GPR15L |
| Staphylococcus epidermidis, Strain M23864:W2 | NWP16 | PF4V1 |
| Staphylococcus warneri D11 | warneriD12 | TFPI2 |
| Catabacter hongkongensis, Strain AB8_9 | NWP299 | LCN1 |
| Staphylococcus epidermidis, Strain NIHLM001 | NWP66 | MUCL3 |
| Staphylococcus pasteuri F11 | pasteuriF11 | PPY |
| Staphylococcus capitis A7 | capitisA7 | RLN2 |
| Streptococcus vestibularis, Strain F0396 | NWP132 | MUCL3 |
| Staphylococcus pasteuri G7 | pasteuriG7 | PPY |
| Staphylococcus capitis B12 | capitisB12 | SDC4 |
| Fusobacterium ulcerans, Strain AC2_8_11 AN FAA D5 7 | NWP140 | SYCN |
| Sporosarcina sp., Strain 2681 | NWP29 | DEFB130B |
| Streptococcus sp., Strain SR4 | NWP130 | MUCL3 |
| Streptococcus mitis, Strain F0392 | NWP26 | LOC644613 |
| Corynebacterium sp., Strain CMW7794 | NWP98 | TFF2 |
| Bacteroides fragilis, Strain CL05T00C42 | NWP287 | DQA2_Epitope-1 |
| Propionibacterium acnes, Strain HL038PA1 | NWP235 | FAM174A |
| Enterococcus faecalis, Strain TX2137 | NWP117 | FAM3B |
| Coprococcus sp., Strain AAb34 | AB34 | SMR3A |
| Staphylococcus capitis A7 | capitisA7 | SLCO2B1 |
| Streptococcus downei, Oral Taxon 594, Strain F0415 | NWP123 | UNQ6190/PRO20217 |
| Staphylococcus pasteuri F12 | pasteuriF12 | CSF2RA |
| Staphylococcus capitis D5 | capitisD5 | PDIA6 |
| Staphylococcus warneri D11 | warneriD12 | PROCR |
| Fusobacterium sp., Strain CM22 | NWP167 | NPY5R |
| Hungatella hathewayi, Strain WAL-18680 | NWP231 | TFF3 |
| Staphylococcus pasteuri G3 | pasteuriG3 | CSF2RA |
| Streptococcus sobrinus, Strain W1703 | NWP85 | CTRB1 |
| Staphylococcus warneri, Strain SK66 | NWP11 | DQA2_Epitope-1 |
| Propionibacterium acnes, Strain HL036PA1 | NWP226 | TMEM119 |
| Micrococcus luteus, Strain SK58 | NWP08 | LOC644613 |
| Staphylococcus warneri, Strain SK66 | NWP11 | F3 |
| Streptococcus parasanguinis, Strain F0449 | NWP114 | C1QL3 |
| Prevotella melaninogenica, Strain D18 | NWP151 | PDIA6 |
| Lactobacillus crispatus, Strain GED7749C | NWP104 | KLRC2 |
| Proteus mirabilis, Strain WGLW6 | NWP59 | TNFRSF10B |
| Staphylococcus aureus, Strain CM05 | NWP83 | PF4V1 |
| Staphylococcus haemolyticus B5 | haemolyticusB5 | SLC8B1 |
| Streptococcus anginosus, Strain F0211 | NWP125 | CTRB1 |
| Oscillibacter sp., Strain KLE-1728 | NWP269 | TXNDC12 |
| Staphylococcus pasteuri F11 | pasteuriF11 | CSF2RA |
| Staphylococcus epidermidis, Strain NIHLM001 | NWP66 | LOC644613 |
| Propionibacterium acnes, Strain HL005PA3 | NWP216 | TMEM119 |
| Lactobacillus jensenii, Strain 208-1 | NWP111 | TFF1 |
| Staphylococcus hominis C10 | hominisC10 | TEPP |
| Streptococcus sobrinus, Strain W1703 | NWP85 | UNQ6190/PRO20217 |
| Streptococcus sp., Strain C150 | NWP121 | CTRB2 |
| Staphylococcus capitis B12 | capitisB12 | OTOS |
| Peptoniphilus lacrimalis, Strain UPII 315-B | NWP261 | PGLYRP1 |
| Propionibacterium acnes, Strain HL045PA1 | NWP265 | TMEM119 |
| Lachnospiraceae sp., Strain DNF00160 | NWP150 | MUCL3 |
| Bacteroidales bacterium, Strain KA00344 | NWP302 | PTPRR |
| Staphylococcus warneri B9 | warneriB9 | RLN2 |
| Propionibacterium acnes, Strain HL060PA1 | NWP239 | OPRK1 |
| Streptococcus sobrinus, Strain W1703 | NWP85 | MUCL3 |
| Staphylococcus capitis B12 | capitisB12 | HRC |
| Bacteroides dorei, Strain 5_1_36/D4 | NWP244 | EPHB2 |
| Dermabacter sp., Strain HFH0086 | NWP92 | KCNMB4 |
| Proteus mirabilis, Strain WGLW6 | NWP59 | CSPG5 |
| Staphylococcus warneri, Strain SK66 | NWP11 | CDSN |
| Streptococcus sobrinus, Strain W1703 | NWP85 | ENDOU |
| Staphylococcus capitis A7 | capitisA7 | TFPI2 |
| Allobaculum sp., Strain NWP324 | Ery128 | SLC22A5 |
| Escherichia coli, Strain 83972 | NWP04 | LOC644613 |
| Prevotella sp., Oral Taxon 472, Strain F0295 | NWP191 | GFY_Epitope-1 |
| Staphylococcus warneri E4 | warneriE4 | TFPI2 |
| Ruminococcus gnavus, Strain NWP325 | RG74 | TFF3 |
| Streptococcus downei, Oral Taxon 594, Strain F0415 | NWP123 | FGF17 |
| Staphylococcus pasteuri C9 | pasteuriC9 | CNPY3 |
| Collinsella aerofaciens, Strain AAB3 | AB3 | COL10A1 |
| Bacteroidales bacterium, Strain KA00344 | NWP302 | DEFB119 |
| Staphylococcus aureus, Strain CM05 | NWP83 | KCNK1 |
| Staphylococcus pasteuri G7 | pasteuriG7 | CSF2RA |
| Finegoldia magna, Strain SY01 | NWP138 | PDIA3 |
| Corynebacterium sp., Strain HFH0082 | NWP61 | PLA2G2E |
| Sporosarcina sp., Strain 2681 | NWP29 | PLA2G2E |
| Staphylococcus warneri B9 | warneriB9 | XG |
| Klebsiella oxytoca, Strain MIT 10-5249 | NWP52 | LYSMD4 |
| Klebsiella oxytoca, Strain MIT 09-7231 | NWP45 | PF4V1 |
| Hungatella hathewayi, Strain WAL-18680 | NWP231 | TFF2 |
| Staphylococcus warneri, Strain SK66 | NWP11 | PODXL2 |
| Staphylococcus lugdunensis, Strain M23590 | NWP14 | PF4V1 |
| Corynebacterium sp., Strain HFH0082 | NWP61 | FAM19A3 |
| Escherichia coli, Strain MS 115-1 | NWP36 | PLA2G2E |
| Clostridium cadaveris, Strain CC40_001C | NWP246 | PDIA6 |
| Arthrobacter albus, Strain DNF00011 | NWP86 | MUCL3 |
| Escherichia coli, Strain MS 110-3 | NWP35 | FAM19A3 |
| Oscillibacter sp., Strain KLE-1728 | NWP269 | MFSD2A |
| Propionibacterium acnes, Strain HL036PA1 | NWP226 | FAM174A |
| Proteus mirabilis, Strain WGLW6 | NWP59 | SFN |
| Prevotella oris, Strain F0302 | NWP159 | TNFRSF10B |
| Streptococcus downei, Oral Taxon 594, Strain F0415 | NWP123 | CSAG1 |
| Staphylococcus capitis B12 | capitisB12 | MMP24 |
| Citrobacter sp., Strain 30_2 | NWP01 | DRAXIN |
| Lactobacillus gasseri, Strain JV-V03 | NWP106 | LOC644613 |
| Escherichia coli, Strain MS 196-1 | NWP42 | LOC644613 |
| Fusobacterium nucleatum subsp. animalis, Oral Taxon 420, Strain F0419 | NWP196 | SOST |
| Sporosarcina sp., Strain 2681 | NWP29 | FAM19A3 |
| Prevotella bivia, Strain GED7760C | NWP233 | MANF |
| Klebsiella oxytoca, Strain MIT 10-5245 | NWP49 | PF4V1 |
| Propionibacterium acnes, Strain HL050PA2 | NWP238 | IGFBP1 |
| Staphylococcus capitis B12 | capitisB12 | T4S19_Epitope-2 |
| Micrococcus luteus, Strain SK58 | NWP08 | DEFB130B |
| Corynebacterium sp., Strain HFH0082 | NWP61 | CCL18 |
| Dorea sp., Strain AAB35 | AB35 | AGR3 |
| Staphylococcus aureus, Strain CM05 | NWP83 | SRGN |
| Escherichia coli, Strain MS 69-1 | NWP39 | LOC644613 |
| Staphylococcus xylosus F2 | xylosusF2 | ENDOU |
| Staphylococcus warneri, Strain SK66 | NWP11 | ADCY5 |
| Fusobacterium sp., Strain CM22 | NWP153 | NPY5R |
| Streptococcus sp., Strain AS20 | NWP116 | LOC644613 |
| Enterococcus faecium, Strain TX0133a04 | NWP44 | PF4V1 |
| Bacteroides sp., Strain AAB9 | AB9 | EPHA2 |
| Staphylococcus saprophyticus E8 | saprophyticusE8 | SLC6A13 |
| Staphylococcus epidermidis C3 | EpiC3 | XG |
| Staphylococcus epidermidis, Strain NIHLM023 | NWP75 | PF4V1 |
| Fusobacterium sp., Strain CM22 | NWP167 | SLC5A8 |
| Acidaminococcus sp., Strain D21 | NWP157 | PDIA3 |
| Catabacter hongkongensis, Strain AB8_9 | NWP299 | CD248 |
| Sporosarcina sp., Strain 2681 | NWP29 | CCL18 |
| Staphylococcus saprophyticus E8 | saprophyticusE8 | IGFBP4 |
| Veillonella sp., Oral Taxon 780, Strain F0422 | NWP268 | MFSD2A |
| Staphylococcus hominis, Strain C80 | NWP25 | LOC644613 |
| Clostridium orbiscindens, Strain 1_3_50AFAA | NWP137 | PDIA3 |
| Staphylococcus aureus, Strain CM05 | NWP83 | CD320 |
| Enterococcus faecium, Strain TX1330 | NWP19 | FAM19A3 |
| Klebsiella oxytoca, Strain MIT 10-5242 | NWP46 | PF4V1 |
| Corynebacterium amycolatum, Strain SK46 | NWP07 | CCL18 |
| Corynebacterium amycolatum, Strain SK46 | NWP07 | DEFB130B |
| Fusobacterium nucleatum subsp. animalis, Oral Taxon 420, Strain F0419 | NWP196 | DKK1 |
| Finegoldia magna, Strain SY01 | NWP138 | PDIA6 |
| Corynebacterium amycolatum, Strain SK46 | NWP07 | FAM19A3 |
| Escherichia coli, Strain MS 198-1 | NWP43 | PF4V1 |
| Propionibacterium acnes, Strain HL044PA1 | NWP237 | TFF3 |
| Streptococcus sanguinis, Strain VMC66 | NWP135 | GCGR |
| Staphylococcus warneri E4 | warneriE4 | PROCR |
| Clostridium difficile, Strain 002-P50-2011 | NWP230 | TXNDC12 |
| Klebsiella oxytoca, Strain MIT 10-5243 | NWP47 | TMEM119 |
| Staphylococcus epidermidis, Strain NIH04008 | NWP77 | LOC644613 |
| Escherichia coli, Strain MS 110-3 | NWP35 | PLA2G2E |
| Shuttleworthia sp., Strain MSX8B | NWP149 | PRND |
| Corynebacterium sp., Strain HFH0082 | NWP61 | DEFB130B |
| Streptococcus sp., Strain ACC21 | NWP134 | MUCL3 |
| Staphylococcus aureus, Strain TCH70 | NWP12 | KCNK1 |
| Bacteroides stercoris, Strain CC31F | NWP270 | TFF3 |
| Staphylococcus aureus, Strain TCH70 | NWP12 | CD320 |
| Ruminococcus gnavus, Strain AAB28 | AB28 | MUCL3 |
| Corynebacterium sp., Strain HFH0082 | NWP61 | NPPC |
| Klebsiella oxytoca, Strain MIT 10-5249 | NWP52 | ICAM1 |
| Oscillibacter sp., Strain KLE-1728 | NWP269 | KCMA1_Epitope-1 |
| Lactobacillus jensenii, Strain EX849587VC06 | NWP109 | TFF3 |
| Enterococcus faecium, Strain TX1330 | NWP19 | DEFB130B |
| Sporosarcina sp., Strain 2681 | NWP29 | NPPC |
| Corynebacterium amycolatum, Strain SK46 | NWP07 | PLA2G2E |
| Klebsiella sp., Strain 4_1_44FAA | NWP21 | TMEM119 |
| Escherichia coli, Strain MS 110-3 | NWP35 | CCL18 |
| Lactobacillus jensenii, Strain EX849587VC06 | NWP109 | DEFB130B |
| Propionibacterium acnes, Strain HL053PA1 | NWP260 | FAM174A |
| Staphylococcus capitis B8 | capitisB8 | PLA2G2E |
| Plesiomonas sp., Strain HPP0020 | NWP62 | LOC644613 |
| Staphylococcus aureus, Strain CM05 | NWP83 | SHISA7 |
| Micrococcus luteus, Strain SK58 | NWP08 | PIANP |
| Ruminococcus gnavus, Strain NWP326 | RG109 | MANF |
| Klebsiella sp., Strain MS 92-3 | NWP40 | PF4V1 |
| Staphylococcus aureus, Strain CM05 | NWP83 | SLC8B1 |
| Propionibacterium acnes, Strain HL036PA2 | NWP227 | FAM174A |
| Enterococcus faecium, Strain TX1330 | NWP19 | NPPA |
| Dorea formicigenerans, Strain AAB36 | AB36 | MANF |
| Sporosarcina sp., Strain 2681 | NWP29 | NPPA |
| Streptococcus mitis, Strain F0392 | NWP26 | CTRB1 |
| Clostridium difficile, Strain 002-P50-2011 | NWP230 | MFSD2A |
| Prevotella sp., Oral Taxon 472, Strain F0295 | NWP191 | SDC1 |
| Lactobacillus gasseri, Strain SV-16A-US | NWP102 | ENDOU |
| Staphylococcus capitis C4 | capitisC4 | PLA2G2E |
| Staphylococcus capitis A7 | capitisA7 | PSORS1C2 |
| Lactobacillus jensenii, Strain EX849587VC06 | NWP109 | FAM19A2 |
| Streptococcus sp., Strain OBRC6 | NWP124 | MUCL3 |
| Prevotella bivia, Strain GED7760C | NWP233 | MUCL3 |
| Streptococcus sobrinus, Strain W1703 | NWP85 | CSAG1 |
| Erysipelotrichaceae sp., Strain 6_1_45 | NWP174 | LOC644613 |
| Streptococcus sp., Strain C150 | NWP121 | PF4V1 |
| Escherichia coli, Strain MS 115-1 | NWP36 | DEFB130B |
| Micrococcus luteus, Strain SK58 | NWP08 | NPPA |
| Streptococcus sp., Strain C150 | NWP121 | MUCL3 |
| Bacteroides stercoris, Strain CC31F | NWP270 | SPINT3 |
| Streptococcus mitis, Strain F0392 | NWP26 | MUCL3 |
| Bacteroidales bacterium, Strain KA00344 | NWP302 | PEBP4 |
| Micrococcus luteus, Strain SK58 | NWP08 | FAM19A3 |
| Blautia sp., Strain AAB30 | AB30 | COL10A1 |
| Enterococcus faecium, Strain ERV165 | NWP81 | FAM19A3 |
| Staphylococcus warneri, Strain SK66 | NWP11 | FAM174A |
| Propionibacterium acnes, Strain HL007PA1 | NWP225 | FAM174A |
| Erysipelotrichaceae sp., Strain 6_1_45 | NWP174 | PLA2G2E |
| Bacteroides stercoris, Strain CC31F | NWP270 | PEBP4 |
| Staphylococcus capitis B12 | capitisB12 | FGFR1 |
| Eggerthella sp., Strain MVA1 | NWP185 | LOC644613 |
| Streptococcus sp., Strain C150 | NWP121 | ENDOU |
| Streptococcus salivarius, Strain SK126 | NWP118 | LOC644613 |
| Escherichia coli, Strain MS 196-1 | NWP42 | FAM19A3 |
| Lactobacillus jensenii, Strain 208-1 | NWP111 | MUCL3 |
| Corynebacterium sp., Strain HFH0082 | NWP61 | NPPA |
| Streptococcus sobrinus, Strain W1703 | NWP85 | FGF17 |
| Enterococcus faecium, Strain E417 | NWP79 | MUCL3 |
| Staphylococcus warneri, Strain SK66 | NWP11 | GAST |
| Escherichia coli, Strain MS 110-3 | NWP35 | DEFB130B |
| Rothia dentocariosa, Strain M567 | NWP23 | TMEM119 |
| Fusobacterium nucleatum subsp. animalis, Oral Taxon 420, Strain F0419 | NWP196 | FAM19A2 |
| Escherichia coli, Strain MS 115-1 | NWP36 | FAM19A3 |
| Escherichia coli, Strain MS 115-1 | NWP36 | CCL18 |
| Staphylococcus saprophyticus E8 | saprophyticusE8 | XG |
| Enterococcus faecium, Strain TX1330 | NWP19 | CCL18 |
| Acidaminococcus sp., Strain D21 | NWP157 | PDIA6 |
| Staphylococcus warneri B9 | warneriB9 | CHGA |
| Oscillibacter sp., Strain KLE 1745 | NWP252 | PDIA6 |
| Staphylococcus lugdunensis, Strain M23590 | NWP14 | TMEM119 |
| Klebsiella oxytoca, Strain MIT 10-5249 | NWP52 | GHRHR |
| Lactobacillus crispatus, Strain GED7749C | NWP104 | GPR37 |
| Lactobacillus parafarraginis, Strain F0439 | NWP105 | FGF17 |
| Prevotella disiens, Strain DNF00882 | NWP177 | SLC6A13 |
| Streptococcus sp., Strain SR1 | NWP122 | CTRB2 |
| Fusobacterium sp., Strain CM22 | NWP167 | SLC29A4 |
| Clostridium orbiscindens, Strain 1_3_50AFAA | NWP137 | PDIA6 |
| Enterococcus faecalis, Strain HH22 | NWP18 | PF4V1 |
| Propionibacterium acnes, Strain HL043PA2 | NWP264 | FAM174A |
| Staphylococcus pasteuri C9 | pasteuriC9 | GPR156 |
| Fusobacterium sp., Strain CM22 | NWP167 | IL15RA |
| Proteus mirabilis, Strain WGLW6 | NWP59 | C1QBP |
| Staphylococcus pasteuri C9 | pasteuriC9 | CSMT1_Epitope-1 |
| Escherichia coli, Strain MS 16-3 | NWP37 | PF4V1 |
| Escherichia coli, Strain MS 85-1 | NWP30 | DEFB130B |
| Prevotella bivia, Strain DNF00188 | NWP193 | AMELX |
| Escherichia coli, Strain MS 200-1 | NWP41 | PF4V1 |
| Staphylococcus warneri B9 | warneriB9 | FGFR1 |
| Enterococcus faecium, Strain ERV165 | NWP81 | DEFB130B |
| Staphylococcus warneri E6 | warneriE6 | TMEM8 |
| Ruminococcus gnavus, Strain NWP325 | RG74 | TFF1 |
| Arthrobacter albus, Strain DNF00011 | NWP86 | SERPINA3 |
| Bacteroides stercoris, Strain CC31F | NWP270 | EPHA5 |
| Streptococcus sp., Strain C150 | NWP121 | LOC644613 |
| Streptococcus sp., Strain ACS2 | NWP115 | PF4V1 |
| Prevotella bivia, Strain DNF00650 | NWP176 | CALU |
| Staphylococcus aureus, Strain TCH70 | NWP12 | SRGN |
| Staphylococcus warneri, Strain SK66 | NWP11 | CHGA |
| Propionibacterium acnes, Strain HL002PA2 | NWP214 | FAM174A |
| Staphylococcus epidermidis, Strain NIH04008 | NWP77 | MUCL3 |
| Escherichia coli, Strain MS 85-1 | NWP30 | FAM19A3 |
| Prevotella oralis, Strain HGA0225 | NWP147 | PDIA6 |
| Staphylococcus warneri D11 | warneriD12 | TMEM8 |
| Escherichia coli, Strain MS 200-1 | NWP41 | TFF2 |
| Enterococcus faecium, Strain TX1330 | NWP19 | PLA2G2E |
| Enterococcus faecium, Strain E417 | NWP79 | FGF17 |
| Lachnospiraceae sp., Strain ICM7 | NWP241 | SPINK9 |
| Klebsiella oxytoca, Strain MIT 10-5244 | NWP48 | PF4V1 |
| Megasphaera micronuciformis, Strain DNF00954 | NWP307 | SMR3A |
| Staphylococcus aureus, Strain TCH70 | NWP12 | KCNK4_Epitope-1 |
| Klebsiella oxytoca, Strain MIT 10-5242 | NWP46 | TMEM119 |
| Proteus mirabilis, Strain WGLW6 | NWP59 | CHGA |
| Fusobacterium sp., Strain CM22 | NWP167 | GP6 |
| Escherichia coli, Strain MS 85-1 | NWP30 | CCL18 |
| Lactobacillus parafarraginis, Strain F0439 | NWP105 | CTRB1 |
| Staphylococcus epidermidis, Strain NIHLM040 | NWP76 | PF4V1 |
| Pseudomonas sp., Strain 2_1_26 | NWP20 | PF4V1 |
| Corynebacterium amycolatum, Strain SK46 | NWP07 | NPPA |
| Streptococcus mitis, Strain F0392 | NWP26 | FAM19A3 |
| Staphylococcus capitis E7 | capitisE7 | PLA2G2E |
| Streptococcus downei, Oral Taxon 594, Strain F0415 | NWP123 | SHISA6 |
| Sporosarcina sp., Strain 2681 | NWP29 | DEFB130A |
| Propionibacterium acnes, Strain HL002PA3 | NWP219 | FAM174A |
| Fusobacterium sp., Strain CM22 | NWP167 | PDPN |
| Staphylococcus caprae, Strain C87 | NWP24 | PF4V1 |
| Lactobacillus jensenii, Strain EX849587VC03 | NWP108 | PGLYRP1 |
| Streptococcus sobrinus, Strain W1703 | NWP85 | RTBDN |
| Streptococcus downei, Oral Taxon 594, Strain F0415 | NWP123 | CTRB2 |
| Proteus mirabilis, Strain WGLW6 | NWP59 | DRAXIN |
| Staphylococcus capitis B12 | capitisB12 | FGFR2 |
| Escherichia coli, Strain MS 21-1 | NWP38 | PF4V1 |
| Plesiomonas sp., Strain HPP0020 | NWP62 | PF4V1 |
| Prevotella melaninogenica, Strain D18 | NWP151 | C3AR_Epitope-3 |
| Staphylococcus epidermidis, Strain W23144 | NWP15 | PF4V1 |
| Escherichia coli, Strain MS 21-1 (AIEC/NWP38) | AIEC | CEACAM1 |
| Escherichia coli, Strain MS 21-1 (AIEC/NWP38) | AIEC | CD55 |
| Staphylococcus capitis B12 | capitisB12 | TENM1 |
| Bacteroidales bacterium, Strain KA00344 | NWP302 | GPC6 |
| Micrococcus luteus, Strain SK58 | NWP08 | PLA2G2E |
| Weissella cibaria, Strain F16_1 | NWP101 | TFF3 |
| Staphylococcus aureus, Strain TCH70 | NWP12 | PF4V1 |
| Propionibacterium acnes, Strain HL045PA1 | NWP265 | FAM174A |
| Hungatella hathewayi, Strain WAL-18680 | NWP231 | SMR3A |
| Escherichia coli, Strain 83972 | NWP04 | CCL18 |
| Propionibacterium acnes, Strain HL043PA2 | NWP264 | TMEM119 |
| Escherichia coli, Strain MS 85-1 | NWP30 | PLA2G2E |
| Staphylococcus capitis D5 | capitisD5 | PDIA3 |
| Citrobacter freundii, Strain 4_7_47CFAA | NWP88 | DRAXIN |
| Corynebacterium sp., Strain CMW7794 | NWP98 | PLA2G2E |
| Allobaculum sp., Strain NWP324 | Ery128 | PF4V1 |
| Lactobacillus parafarraginis, Strain F0439 | NWP105 | CTRB2 |
| Streptococcus sobrinus, Strain W1703 | NWP85 | PDGFB |
| Clostridium difficile, Strain 002-P50-2011 | NWP230 | KCMA1_Epitope-1 |
| Clostridium sp., Strain MSTE9 | NWP253 | SPINK9 |
| Staphylococcus epidermidis E11 | EpiE11 | XG |
| Citrobacter freundii, Strain 4_7_47CFAA | NWP28 | DRAXIN |
| Staphylococcus epidermidis, Strain NIHLM001 | NWP66 | UNQ6190/PRO20217 |
| Staphylococcus warneri E6 | warneriE6 | ACTR3C |
| Bacteroides stercoris, Strain CC31F | NWP270 | DQA2_Epitope-1 |
| Staphylococcus epidermidis, Strain NIH05001 | NWP78 | PF4V1 |
| Streptococcus mitis, Strain F0392 | NWP26 | CCL18 |
| Propionibacterium acnes, Strain HL002PA2 | NWP214 | TMEM119 |
| Enterococcus faecalis, Strain TX2137 | NWP117 | SCNAA_Epitope-3 |
| Oscillibacter sp., Strain KLE-1728 | NWP269 | PDIA2 |
| Staphylococcus pasteuri E5 | pasteuriE5 | GYPA |
| Escherichia coli, Strain MS 110-3 | NWP35 | NPPA |
| Lactobacillus jensenii, Strain EX849587VC06 | NWP109 | PF4V1 |
| Bacteroides stercoris, Strain CC31F | NWP270 | TFF2 |
| Propionibacterium acnes, Strain HL020PA1 | NWP221 | FAM174A |
| Prevotella bivia, Strain DNF00650 | NWP176 | AMELX |
| Prevotella disiens, Strain DNF00882 | NWP177 | CLU |
| Sporosarcina sp., Strain 2681 | NWP29 | PIANP |
| Clostridium orbiscindens, Strain CC43_001K | NWP259 | PDIA6 |
| Lactobacillus crispatus, Strain GED7749C | NWP104 | KLRC1 |
| Enterococcus faecium, Strain ERV165 | NWP81 | PLA2G2E |
| Propionibacterium acnes, Strain HL046PA2 | NWP266 | TMEM119 |
| Citrobacter sp., Strain 30_2 | NWP01 | CCL21 |
| Peptoniphilus sp., Oral Taxon 386, Strain F0131 | NWP255 | IGFBP6 |
| Staphylococcus warneri E6 | warneriE6 | PLA2G2A |
| Streptococcus mitis, Strain F0392 | NWP26 | DEFB130B |
| Staphylococcus saprophyticus E8 | saprophyticusE8 | MTNR1B |
| Staphylococcus aureus, Strain CM05 | NWP83 | KCNK4_Epitope-1 |
| Streptococcus anginosus, Strain F0211 | NWP125 | CTRB2 |
| Plesiomonas sp., Strain HPP0020 | NWP62 | PLA2G2E |
| Erysipelotrichaceae sp., Strain 6_1_45 | NWP181 | LOC644613 |
| Fusobacterium nucleatum subsp. animalis, Oral Taxon 420, Strain F0419 | NWP196 | CCL21 |
| Escherichia coli, Strain MS 69-1 | NWP39 | FAM19A3 |
| Acidaminococcus sp., Strain D21 | NWP157 | TXNDC12 |
| Enterococcus faecium, Strain TX1330 | NWP19 | NPPC |
| Prevotella nigrescens, Strain F0103 | NWP145 | TTYH1_Epitope-3 |
| Bacteroides stercoris, Strain CC31F | NWP270 | SCN9A_Epitope-3 |
| Enterococcus faecalis, Strain TX2137 | NWP117 | ENDOU |
| Staphylococcus warneri F1 | warneriF1 | PSORS1C2 |
| Propionibacterium acnes, Strain HL005PA1 | NWP224 | TMEM119 |
| Deinococcus grandis, Strain SK125 | NWP89 | PLA2G2E |
| Propionibacterium acnes, Strain HL005PA4 | NWP220 | FAM174A |
| Citrobacter sp., Strain 30_2 | NWP01 | FAM19A2 |
| Lachnospiraceae bacterium, Strain AAB23 | AB23 | SMR3A |
| Propionibacterium acnes, Strain HL056PA1 | NWP267 | TMEM119 |
| Staphylococcus hominis, Strain SK119 | NWP10 | PF4V1 |
| Staphylococcus pasteuri E5 | pasteuriE5 | FAM174A |
| Erysipelotrichaceae sp., Strain 6_1_45 | NWP181 | PLA2G2E |
| Sporosarcina sp., Strain 2681 | NWP29 | SMR3A |
| Streptococcus mitis, Strain F0392 | NWP26 | PLA2G2E |
| Streptococcus sp., Strain BS21 | NWP131 | ENDOU |
| Corynebacterium amycolatum, Strain SK46 | NWP07 | PIANP |
| Propionibacterium acnes, Strain HL005PA2 | NWP215 | TMEM119 |
| Lactobacillus crispatus, Strain JV-V01 | NWP107 | PDIA2 |
| Corynebacterium sp., Strain HFH0082 | NWP61 | PIANP |
| Staphylococcus pasteuri E5 | pasteuriE5 | CSF2RA |
| Escherichia coli, Strain MS 200-1 | NWP41 | LOC644613 |
| Escherichia coli, Strain MS 85-1 | NWP30 | NPPC |
| Proteus mirabilis, Strain WGLW6 | NWP59 | GPR1 |
| Erysipelotrichaceae sp., Strain 6_1_45 | NWP174 | NPPC |
| Staphylococcus epidermidis, Strain NIHLM015 | NWP71 | PF4V1 |
| Staphylococcus warneri D11 | warneriD12 | ACTR3C |
| Propionibacterium sp., Strain 5_U_42AFAA | NWP300 | TMEM119 |
| Erysipelotrichaceae sp., Strain 6_1_45 | NWP174 | CCL18 |
| Megasphaera sp., Strain DNF00912 | NWP296 | SMR3A |
| Bacteroides dorei, Strain 5_1_36/D4 | NWP244 | PTPRJ |
| Staphylococcus aureus, Strain MN8 | NWP17 | SRGN |
| Plesiomonas sp., Strain HPP0020 | NWP62 | CCL18 |
| Enterococcus faecium, Strain TX1330 | NWP19 | PIANP |
| Klebsiella oxytoca, Strain MIT 10-5249 | NWP52 | ACVR2B |
| Streptococcus mitis, Strain F0392 | NWP26 | NPPA |
| Prevotella bivia, Strain DNF00188 | NWP193 | CALU |
| Staphylococcus caprae, Strain C87 | NWP24 | LOC644613 |
| Lactobacillus crispatus, Strain JV-V01 | NWP107 | TFF1 |
| Corynebacterium sp., Strain HFH0082 | NWP61 | SMR3A |
| Staphylococcus warneri B9 | warneriB9 | PPY |
| Klebsiella oxytoca, Strain MIT 09-7231 | NWP45 | TMEM119 |
| Lachnospiraceae bacterium, Strain AAB19 | AB19 | SMR3A |
| Erysipelotrichaceae sp., Strain 6_1_45 | NWP181 | CCL18 |
| Escherichia coli, Strain MS 115-1 | NWP36 | PIANP |
| Staphylococcus epidermidis, Strain BCM0060 | NWP13 | PF4V1 |
| Escherichia sp., Strain 3_2_53FAA | NWP02 | LOC644613 |
| Staphylococcus cohnii G11 | cohniiG11 | SLC8B1 |
| Achromobacter xylosoxidans, Strain C54 | NWP22 | TMEM119 |
| Citrobacter freundii, Strain 4_7_47CFAA | NWP28 | IGFBP5 |
| Oscillibacter sp., Strain KLE-1728 | NWP269 | PDIA3 |
| Staphylococcus epidermidis, Strain NIHLM001 | NWP66 | FGF17 |
| Bacteroides sp., Strain AAB9 | AB9 | EPHA5 |
| Klebsiella sp., Strain MS 92-3 | NWP40 | TMEM119 |
| Escherichia coli, Strain MS 85-1 | NWP30 | SMR3A |
| Citrobacter freundii, Strain 4_7_47CFAA | NWP88 | IGFBP5 |
| Staphylococcus warneri B9 | warneriB9 | AT134_Epitope-2 |
| Propionibacterium acnes, Strain HL027PA2 | NWP218 | FAM174A |
| Staphylococcus aureus, Strain TCH70 | NWP12 | MSMB |
| Lactobacillus gasseri, Strain 224-1 | NWP112 | PF4V1 |
| Lactobacillus jensenii, Strain 269-3 | NWP110 | TMEM149 |
| Staphylococcus pasteuri C9 | pasteuriC9 | PLA2G2A |
| Propionibacterium acnes, Strain HL036PA2 | NWP227 | TMEM119 |
| Bacteroides dorei, Strain 5_1_36/D4 | NWP244 | BTN1A1 |
| Erysipelotrichaceae sp., Strain 6_1_45 | NWP181 | COL10A1 |
| Fusobacterium sp., Strain CM22 | NWP167 | CSF3 |
| Prevotella sp., Oral Taxon 317, Strain F0108 | NWP173 | CSPG5 |
| Micrococcus luteus, Strain SK58 | NWP08 | SMR3A |
| Streptococcus sp., Strain C150 | NWP121 | PDGFB |
| Bacillus sp., Strain DNF00233 | NWP90 | PLA2G2E |
| Propionibacterium acnes, Strain HL013PA1 | NWP209 | TMEM119 |
| Prevotella timonensis, Strain CRIS 5C-B1 | NWP156 | TXNDC12 |
| Lactobacillus rhamnosus, Strain LMS2-1 | NWP97 | TFF1 |
| Propionibacterium acnes, Strain HL056PA1 | NWP267 | FAM174A |
| Streptococcus mitis, Strain F0392 | NWP26 | NPPC |
| Escherichia coli, Strain MS 85-1 | NWP30 | PIANP |
| Propionibacterium acnes, Strain HL020PA1 | NWP221 | TMEM119 |
| Escherichia coli, Strain 83972 | NWP04 | PLA2G2E |
| Bacteroides stercoris, Strain CC31F | NWP270 | TEX29_Epitope-1 |
| Staphylococcus aureus, Strain TCH70 | NWP12 | SLC8B1 |
| Plesiomonas sp., Strain HPP0020 | NWP62 | NPPA |
| Staphylococcus pasteuri E5 | pasteuriE5 | HCG22 |
| Propionibacterium acnes, Strain HL025PA2 | NWP210 | OPRK1 |
| Blautia sp., Strain AAB30 | AB30 | TREML1 |
| Streptococcus sp., Strain SR1 | NWP122 | ENDOU |
| Escherichia coli, Strain MS 85-1 | NWP30 | NPPA |
| Proteus mirabilis, Strain WGLW6 | NWP59 | SGCA |
| Dermabacter sp., Strain HFH0086 | NWP92 | LRRC8D |
| Clostridium sp., Strain 7_2_43FAA | NWP243 | PDIA6 |
| Sporosarcina sp., Strain 2681 | NWP29 | IGFBP5 |
| Rothia dentocariosa, Strain M567 | NWP23 | PF4V1 |
| Fusobacterium nucleatum subsp. animalis, Oral Taxon 420, Strain F0419 | NWP196 | CCL5 |
| Fusobacterium sp., Strain CM21 | NWP166 | LOC644613 |
| Proteus mirabilis, Strain WGLW6 | NWP59 | PODXL2 |
| Propionibacterium acnes, Strain HL030PA2 | NWP207 | OPRK1 |
| Escherichia coli, Strain MS 69-1 | NWP39 | PF4V1 |
| Staphylococcus warneri B9 | warneriB9 | F3 |
| Enterococcus faecium, Strain ERV165 | NWP81 | PIANP |
| Escherichia coli, Strain 83972 | NWP04 | FAM19A3 |
| Bacteroides sp., Strain D20 | NWP288 | ENDOU |
| Enterococcus faecalis, Strain HH22 | NWP18 | TMEM119 |
| Fusobacterium nucleatum subsp. animalis, Oral Taxon 420, Strain F0419 | NWP196 | IGFBP5 |
| Staphylococcus pasteuri G3 | pasteuriG3 | GYPA |
| Pseudomonas sp., Strain HPB0071 | NWP65 | PF4V1 |
| Eggerthella sp., Strain MVA1 | NWP185 | CCL18 |
| Bacteroides fragilis, Strain CL05T00C42 | NWP287 | ENDOU |
| Corynebacterium amycolatum, Strain SK46 | NWP07 | SMR3A |
| Escherichia coli, Strain MS 110-3 | NWP35 | PIANP |
| Klebsiella oxytoca, Strain MIT 10-5244 | NWP48 | TMEM119 |
| Megasphaera sp., Strain DNF00912 | NWP296 | SPINK9 |
| Corynebacterium amycolatum, Strain SK46 | NWP07 | COL10A1 |
| Staphylococcus pasteuri C9 | pasteuriC9 | TMEM178B |
| Staphylococcus pasteuri G3 | pasteuriG3 | HCG22 |
| Proteus mirabilis, Strain WGLW6 | NWP59 | AGRN |
| Lactobacillus crispatus, Strain 125-2-CHN | NWP99 | ENDOU |
| Escherichia coli, Strain MS 69-1 | NWP39 | PLA2G2E |
| Propionibacterium acnes, Strain HL027PA2 | NWP218 | TMEM119 |
| Propionibacterium acnes, Strain HL007PA1 | NWP225 | TMEM119 |
| Staphylococcus epidermidis, Strain NIHLM040 | NWP76 | XG |
| Clostridium sp., Strain MSTE9 | NWP253 | IGFBP6 |
| Streptococcus mitis, Strain F0392 | NWP26 | PIANP |
| Bacteroides stercoris, Strain CC31F | NWP270 | AGRN |
| Fusobacterium nucleatum, Strain CTI-07 | NWP190 | ENDOU |
| Lactobacillus parafarraginis, Strain F0439 | NWP105 | UNQ6190/PRO20217 |
| Streptococcus sp., Strain CM7 | NWP133 | MUCL3 |
| Propionibacterium acnes, Strain HL053PA1 | NWP260 | TMEM119 |
| Staphylococcus warneri C2 | warneriC2 | C2orf40 |
| Staphylococcus epidermidis, Strain NIHLM001 | NWP66 | CCL2 |
| Streptococcus sp., Strain OBRC6 | NWP124 | RTBDN |
| Coprococcus sp., Strain AAb34 | AB34 | PLA2G2E |
| Corynebacterium sp., Strain HFH0082 | NWP61 | COL10A1 |
| Propionibacterium acidifaciens, Oral Taxon 191, Strain F0233 | NWP263 | SPINK9 |
| Propionibacterium acnes, Strain HL037PA1 | NWP208 | FAM174A |
| Propionibacterium acnes, Strain HL002PA1 | NWP206 | FAM174A |
| Propionibacterium acnes, Strain HL038PA1 | NWP235 | TMEM119 |
| Propionibacterium acnes, Strain HL053PA2 | NWP236 | FAM174A |
| Fusobacterium sp., Strain CM22 | NWP167 | PSORS1C2 |
| Staphylococcus warneri D11 | warneriD12 | DKK4 |
| Clostridium orbiscindens, Strain 1_3_50AFAA | NWP137 | GPR25 |
| Propionibacterium acnes, Strain HL030PA2 | NWP207 | FAM174A |
| Staphylococcus pasteuri E5 | pasteuriE5 | KLK13 |
| Streptococcus gallolyticus subsp. gallolyticus, Strain TX20005 | NWP27 | CST6 |
| Staphylococcus capitis A7 | capitisA7 | EPO |
| Enterococcus faecium, Strain ERV165 | NWP81 | NPPC |
| Lactobacillus jensenii, Strain 208-1 | NWP111 | ASIP |
| Staphylococcus pasteuri E1 | pasteuriE1 | PPY |
| Proteus mirabilis, Strain WGLW6 | NWP59 | DKK1 |
| Enterococcus faecium, Strain TX1330 | NWP19 | COL10A1 |
| Propionibacterium acnes, Strain HL110PA3 | NWP297 | SMR3A |
| Streptococcus vestibularis, Strain F0396 | NWP132 | UNQ6190/PRO20217 |
| Proteus mirabilis, Strain WGLW6 | NWP59 | SYNDIG1L |
| Escherichia coli, Strain MS 115-1 | NWP36 | NPPA |
| Prevotella bivia, Strain DNF00188 | NWP193 | MANF |
| Prevotella histicola, Strain F0411 | NWP144 | PRRG1 |
| Escherichia coli, Strain MS 145-7 | NWP33 | TNFSF9 |
| Klebsiella oxytoca, Strain MIT 10-5249 | NWP52 | AHSG |
| Pseudomonas sp., Strain 2_1_26 | NWP20 | TMEM119 |
| Staphylococcus saprophyticus F6 | saprophyticusF6 | PDGFB |
| Propionibacterium acnes, Strain HL013PA2 | NWP217 | TMEM119 |
| Bacteroidales bacterium, Strain KA00344 | NWP302 | CD248 |
| Staphylococcus saprophyticus E8 | saprophyticusE8 | GPR17 |
| Micrococcus luteus, Strain SK58 | NWP08 | CCL18 |
| Capnocytophaga sputigena, Strain CC21_001D | NWP163 | CST1 |
| Enterococcus faecium, Strain ERV165 | NWP81 | SMR3A |
| Enterococcus faecalis, Strain TUSoD Ef11 | NWP05 | PF4V1 |
| Staphylococcus capitis B12 | capitisB12 | P2RX5 |
| Staphylococcus warneri D11 | warneriD12 | PLA2G3 |
| Staphylococcus saprophyticus E8 | saprophyticusE8 | SHISA7 |
| Oscillibacter sp., Strain KLE-1728 | NWP269 | CXCR4 |
| Streptococcus sp., Strain ACS2 | NWP115 | FAM3C |
| Eggerthella sp., Strain MVA1 | NWP185 | PLA2G2E |
| Citrobacter freundii, Strain 4_7_47CFAA | NWP28 | CCL21 |
| Staphylococcus pasteuri G3 | pasteuriG3 | KLK13 |
| Ruminococcus gnavus, Strain NWP325 | RG74 | CD7 |
| Staphylococcus capitis C11 | capitisC11 | PDIA3 |
| Propionibacterium acnes, Strain HL060PA1 | NWP239 | FAM174A |
| Clostridium difficile, Strain 002-P50-2011 | NWP230 | PDIA6 |
| Enterococcus faecium, Strain ERV102 | NWP80 | MUCL3 |
| Propionibacterium acnes, Strain HL025PA1 | NWP204 | OPRK1 |
| Propionibacterium acnes, Strain HL036PA3 | NWP222 | FAM174A |
| Streptococcus sp., Strain SR1 | NWP122 | TMEM149 |
| Lactobacillus jensenii, Strain EX849587VC06 | NWP109 | SHISA2 |
| Fusobacterium sp., Strain CM22 | NWP153 | SGCA |
| Enterococcus faecium, Strain E417 | NWP79 | UNQ6190/PRO20217 |
| Sporosarcina sp., Strain 2681 | NWP29 | CHRDL2 |
| Staphylococcus epidermidis, Strain NIHLM020 | NWP74 | LOC644613 |
| Staphylococcus epidermidis F6 | EpiF6 | COL10A1 |
| Arthrobacter albus, Strain DNF00011 | NWP86 | LTB |
| Staphylococcus pasteuri F11 | pasteuriF11 | HCG22 |
| Staphylococcus pasteuri G7 | pasteuriG7 | GYPA |
| Streptococcus sp., Strain SR4 | NWP130 | UNQ6190/PRO20217 |
| Streptococcus sobrinus, Strain W1703 | NWP85 | PIANP |
| Streptococcus sp., Strain BS29a | NWP129 | FAM168B |
| Proteus mirabilis, Strain WGLW6 | NWP59 | LDLRAD3 |
| Streptococcus sobrinus, Strain W1703 | NWP85 | SHISA6 |
| Lactobacillus crispatus, Strain JV-V01 | NWP107 | PF4V1 |
| Eubacterium sp., Strain AS15 | NWP254 | SPINK9 |
| Escherichia coli, Strain MS 145-7 | NWP33 | PF4V1 |
| Veillonella sp., Oral Taxon 780, Strain F0422 | NWP268 | TXNDC12 |
| Staphylococcus warneri B9 | warneriB9 | SLC24A3 |
| Staphylococcus warneri D11 | warneriD12 | EPHB3 |
| Staphylococcus hominis C10 | hominisC10 | XG |
| Staphylococcus warneri E6 | warneriE6 | RTN4R |
| Fusobacterium sp., Strain CM22 | NWP167 | TRABD2B |
| Bacteroides sp., Strain 4_1_36 | NWP312 | PRR27 |
| Collinsella aerofaciens, Strain AAB3 | AB3 | SMR3A |
| Staphylococcus warneri, Strain SK66 | NWP11 | MCFD2 |
| Escherichia coli, Strain MS 124-1 | NWP32 | TMEM119 |
| Ruminococcus gnavus, Strain NWP326 | RG109 | AGER |
| Staphylococcus warneri E6 | warneriE6 | DKK4 |
| Bacteroides stercoris, Strain CC31F | NWP270 | CSF2 |
| Prevotella bivia, Strain DNF00650 | NWP176 | OTOS |
| Proteus mirabilis, Strain WGLW6 | NWP59 | GAST |
| Staphylococcus warneri B9 | warneriB9 | SDC4 |
| Staphylococcus warneri E4 | warneriE4 | TMEM8 |
| Erysipelotrichaceae sp., Strain 6_1_45 | NWP181 | SMR3A |
| Propionibacterium acnes, Strain HL030PA1 | NWP212 | FAM174A |
| Propionibacterium acnes, Strain HL036PA3 | NWP222 | TMEM119 |
| Lactobacillus reuteri, Strain CF48-3A | NWP100 | TFF2 |
| Blautia sp., Strain AAB32 | AB32 | TFF2 |
| Prevotella bivia, Strain DNF00650 | NWP176 | PMCH |
| Enterococcus faecium, Strain ERV99 | NWP82 | PF4V1 |
| Staphylococcus warneri E6 | warneriE6 | EPHB3 |
| Escherichia coli, Strain MS 124-1 | NWP32 | SMR3A |
| Dorea sp., Strain AAB35 | AB35 | PDIA2 |
| Enterococcus faecium, Strain ERV165 | NWP81 | DEFB130A |
| Fusobacterium sp., Strain CM22 | NWP167 | OR5T3_Epitope-1 |
| Staphylococcus saprophyticus E8 | saprophyticusE8 | RLN2 |
| Staphylococcus epidermidis, Strain NIH04008 | NWP77 | FGF17 |
| Staphylococcus capitis A7 | capitisA7 | TMEM8 |
| Propionibacterium acnes, Strain HL005PA1 | NWP224 | FAM174A |
| Escherichia coli, Strain MS 196-1 | NWP42 | DEFB130B |
| Lactobacillus jensenii, Strain 208-1 | NWP111 | CTRB1 |
| Lactobacillus crispatus, Strain GED7749C | NWP104 | BPIFA3 |
| Staphylococcus pasteuri E5 | pasteuriE5 | PODXL2 |
| Prevotella bivia, Strain DNF00188 | NWP193 | PMCH |
| Staphylococcus warneri E6 | warneriE6 | SFRP4 |
| Prevotella nigrescens, Strain F0103 | NWP145 | SLC22A5 |
| Staphylococcus epidermidis, Strain NIHLM001 | NWP66 | CSAG1 |
| Lactobacillus jensenii, Strain 208-1 | NWP111 | CTRB2 |
| Staphylococcus warneri E6 | warneriE6 | DCN |
| Escherichia coli, Strain MS 196-1 | NWP42 | PLA2G2E |
| Escherichia coli, Strain MS 69-1 | NWP39 | DEFB130B |
| Bacteroides fragilis, Strain CL03T12C07 | NWP281 | ENDOU |
| Staphylococcus warneri, Strain SK66 | NWP11 | CP |
| Prevotella veroralis, Oral Taxon 572, Strain F0319 | NWP155 | PDIA3 |
| Staphylococcus saprophyticus G1 | saprophyticusG1 | PDGFB |
| Staphylococcus warneri B9 | warneriB9 | P2RX5 |
| Megasphaera sp., Strain DNF00912 | NWP296 | PDIA6 |
| Lactobacillus parafarraginis, Strain F0439 | NWP105 | RTBDN |
| Staphylococcus pasteuri G7 | pasteuriG7 | HCG22 |
| Escherichia coli, Strain MS 115-1 | NWP36 | NPPC |
| Staphylococcus warneri D11 | warneriD12 | DCN |
| Citrobacter freundii, Strain 4_7_47CFAA | NWP88 | FAM19A2 |
| Bacteroides sp., Strain D2 | NWP192 | PLA2G2E |
| Lachnospiraceae bacterium, Strain AAB19 | AB19 | SEMA6C |
| Collinsella aerofaciens, Strain AAB3 | AB3 | PRR27 |
| Streptococcus sp., Strain SR1 | NWP122 | C3AR_Epitope-3 |
| Staphylococcus pasteuri F11 | pasteuriF11 | KLK13 |
| Staphylococcus pasteuri F12 | pasteuriF12 | HCG22 |
| Bacteroides sp., Strain D2 | NWP192 | CCL18 |
| Klebsiella oxytoca, Strain MIT 10-5245 | NWP49 | TMEM119 |
| Citrobacter freundii, Strain 4_7_47CFAA | NWP28 | FAM19A2 |
| Enterococcus faecium, Strain ERV102 | NWP80 | FAM19A3 |
| Staphylococcus warneri B9 | warneriB9 | TMPRSS11D |
| Oscillibacter sp., Strain KLE-1728 | NWP269 | P4HB |
| Staphylococcus saprophyticus E8 | saprophyticusE8 | PSORS1C2 |
| Escherichia coli, Strain MS 110-3 | NWP35 | DEFB130A |
| Streptococcus sp., Strain C150 | NWP121 | FGF17 |
| Erysipelotrichaceae sp., Strain 6_1_45 | NWP181 | DEFB130B |
| Eubacterium sp., Strain AS15 | NWP254 | IGFBP6 |
| Staphylococcus epidermidis, Strain NIHLM023 | NWP75 | TMEM119 |
| Escherichia coli, Strain MS 110-3 | NWP35 | NPPC |
| Escherichia coli, Strain MS 69-1 | NWP39 | NPPA |
| Klebsiella sp., Strain OBRC7 | NWP60 | TMEM119 |
| Bacteroidales bacterium, Strain KA00344 | NWP302 | C1QTNF2 |
| Klebsiella pneumoniae subsp. pneumoniae, Strain WGLW5 | NWP55 | TMEM119 |
| Staphylococcus epidermidis, Strain M23864:W2 | NWP16 | TMEM119 |
| Staphylococcus capitis B12 | capitisB12 | C2orf40 |
| Staphylococcus epidermidis, Strain NIH04008 | NWP77 | PF4V1 |
| Staphylococcus warneri E4 | warneriE4 | ACTR3C |
| Prevotella oralis, Strain HGA0225 | NWP147 | GPR25 |
| Rothia mucilaginosa, Strain CC87LB | NWP84 | PF4V1 |
| Bacteroides fragilis, Strain CL05T00C42 | NWP287 | FKBP2 |
| Peptoniphilus sp., Oral Taxon 386, Strain F0131 | NWP255 | SPINK9 |
| Enterococcus faecium, Strain E417 | NWP79 | CSAG1 |
| Fusobacterium sp., Strain CM22 | NWP167 | GYPA |
| Lactobacillus parafarraginis, Strain F0439 | NWP105 | CSAG1 |
| Enterococcus faecium, Strain ERV165 | NWP81 | CCL18 |
| Lactobacillus jensenii, Strain EX849587VC03 | NWP108 | FAM19A2 |
| Prevotella denticola, Strain DNF00960 | NWP179 | MUCL3 |
| Lactobacillus jensenii, Strain 208-1 | NWP111 | UNQ6190/PRO20217 |
| Escherichia coli, Strain MS 200-1 | NWP41 | DEFB130B |
| Staphylococcus epidermidis, Strain NIH04008 | NWP77 | UNQ6190/PRO20217 |
| Clostridium aldenense, Strain WAL-18727 | NWP314 | PRR27 |
| Staphylococcus warneri B9 | warneriB9 | GPR15L |
| Propionibacterium acnes, Strain HL053PA2 | NWP236 | OPRK1 |
| Escherichia coli, Strain MS 57-2 | NWP34 | PF4V1 |
| Staphylococcus hominis, Strain C80 | NWP25 | PIANP |
| Staphylococcus pasteuri G3 | pasteuriG3 | PODXL2 |
| Propionibacterium acnes, Strain HL005PA4 | NWP220 | OPRK1 |
| Propionibacterium acnes, Strain HL013PA2 | NWP217 | FAM174A |
| Enterococcus faecium, Strain TX1330 | NWP19 | DEFB130A |
| Prevotella oris, Strain F0302 | NWP159 | PDIA6 |
| Staphylococcus pasteuri F12 | pasteuriF12 | KLK13 |
| Proteus mirabilis, Strain WGLW6 | NWP59 | THBD |
| Escherichia coli, Strain 83972 | NWP04 | PIANP |
| Streptococcus anginosus, Strain F0211 | NWP125 | MUCL3 |
| Staphylococcus warneri E6 | warneriE6 | PLA2G3 |
| Enterococcus faecium, Strain ERV165 | NWP81 | NPPA |
| Lachnospiraceae bacterium, Strain AAB19 | AB19 | SYCN |
| Enterococcus faecium, Strain ERV102 | NWP80 | UNQ6190/PRO20217 |
| Peptoniphilus lacrimalis, Strain DNF00528 | NWP304 | SMR3A |
| Staphylococcus epidermidis, Strain NIH05001 | NWP78 | TMEM119 |
| Propionibacterium acidifaciens, Oral Taxon 191, Strain F0233 | NWP263 | FGF1 |
| Proteus mirabilis, Strain WGLW6 | NWP59 | TMG2_Epitope-1 |
| Staphylococcus pasteuri F12 | pasteuriF12 | GYPA |
| Staphylococcus epidermidis, Strain W23144 | NWP15 | TMEM119 |
| Erysipelotrichaceae sp., Strain 6_1_45 | NWP181 | NPPC |
| Proteus mirabilis, Strain WGLW6 | NWP59 | EPHA2 |
| Propionibacterium acnes, Strain HL059PA2 | NWP251 | TMEM119 |
| Enterococcus faecium, Strain ERV102 | NWP80 | FGF17 |
| Corynebacterium amycolatum, Strain SK46 | NWP07 | NPPC |
| Fusobacterium sp., Oral Taxon 370, Strain F0437 | NWP143 | VSTM2B |
| Staphylococcus warneri E6 | warneriE6 | EXOC3-AS1 |
| Klebsiella oxytoca, Strain MIT 10-5248 | NWP51 | TMEM119 |
| Escherichia coli, Strain MS 196-1 | NWP42 | CCL18 |
| Staphylococcus pasteuri C9 | pasteuriC9 | GPHB5 |
| Klebsiella pneumoniae subsp. pneumoniae, Strain WGLW1 | NWP56 | LOC644613 |
| Staphylococcus epidermidis C3 | EpiC3 | PMCH |
| Micrococcus luteus, Strain SK58 | NWP08 | PRR27 |
| Eubacterium sp., Strain AS15 | NWP254 | TMEM119 |
| Escherichia coli, Strain MS 69-1 | NWP39 | PIANP |
| Staphylococcus hominis B3 | hominisB3 | LOC644613 |
| Proteus mirabilis, Strain WGLW6 | NWP59 | SPOCK1 |
| Staphylococcus epidermidis, Strain E13A | NWP63 | PF4V1 |
| Prevotella melaninogenica, Strain D18 | NWP151 | GPR25 |
| Klebsiella oxytoca, Strain MIT 10-5250 | NWP53 | TMEM119 |
| Enterococcus faecalis, Strain TX2137 | NWP117 | PDGFB |
| Staphylococcus capitis C4 | capitisC4 | SMR3A |
| Klebsiella oxytoca, Strain MIT 10-5248 | NWP51 | PF4V1 |
| Propionibacterium sp., Strain 5_U_42AFAA | NWP300 | FAM174A |
| Bacteroides stercoris, Strain CC31F | NWP270 | SYCN |
| Staphylococcus capitis C12 | capitisC12 | PLA2G2E |
| Propionibacterium acnes, Strain HL050PA3 | NWP249 | FAM174A |
| Clostridiales bacterium, Strain 3_1_39B/D5 | NWP240 | PF4V1 |
| Escherichia coli, Strain MS 85-1 | NWP30 | DEFB130A |
| Corynebacterium amycolatum, Strain SK46 | NWP07 | FAM19A2 |
| Prevotella bivia, Strain DNF00188 | NWP193 | AMELY |
| Eggerthella sp., Strain MVA1 | NWP185 | COL10A1 |
| Klebsiella sp., Strain 1_1_55 | NWP03 | TMEM119 |
| Erysipelotrichaceae sp., Strain 6_1_45 | NWP181 | FAM19A3 |
| Propionibacterium acnes, Strain HL005PA2 | NWP215 | FAM174A |
| Streptococcus sp., Strain C150 | NWP121 | RTBDN |
| Escherichia coli, Strain MS 196-1 | NWP42 | PIANP |
| Micrococcus luteus, Strain SK58 | NWP08 | KLK10 |
| Staphylococcus saprophyticus D1 | saprophyticusD1 | PDGFB |
| Staphylococcus warneri E6 | warneriE6 | CNPY3 |
| Clostridium sp., Strain 7_2_43FAA | NWP243 | ENDOU |
| Staphylococcus warneri D9 | warneriD9 | CTRB1 |
| Streptococcus sp., Strain C150 | NWP121 | TMEM119 |
| Blautia sp., Strain AAB31 | AB31 | MUCL3 |
| Corynebacterium sp., Strain HFH0082 | NWP61 | SEMG1 |
| Prevotella disiens, Strain DNF00882 | NWP177 | CFC1 |
| Erysipelotrichaceae sp., Strain 6_1_45 | NWP181 | PIANP |
| Staphylococcus warneri D11 | warneriD12 | CDSN |
| Sporosarcina sp., Strain 2681 | NWP29 | PRND |
| Eggerthella sp., Strain MVA1 | NWP185 | IGFBP5 |
| Escherichia coli, Strain MS 124-1 | NWP32 | GPR17 |
| Fusobacterium nucleatum, Strain CTI-07 | NWP190 | IGFBP5 |
| Prevotella sp., Oral Taxon 472, Strain F0295 | NWP191 | CSPG5 |
| Enterococcus faecium, Strain E417 | NWP79 | FAM19A3 |
| Citrobacter sp., Strain 30_2 | NWP01 | IGFBP5 |
| Prevotella bivia, Strain DNF00650 | NWP176 | AMELY |
| Staphylococcus epidermidis, Strain NIHLM067 | NWP68 | PF4V1 |
| Bifidobacterium longum subsp. longum, Strain 44B | NWP308 | SMR3A |
| Fusobacterium sp., Strain CM21 | NWP166 | PIANP |
| Enterococcus faecium, Strain ERV165 | NWP81 | FAM19A2 |
| Streptococcus downei, Oral Taxon 594, Strain F0415 | NWP123 | PIANP |
| Staphylococcus warneri E6 | warneriE6 | OPN3 |
| Propionibacterium acidifaciens, Oral Taxon 191, Strain F0233 | NWP263 | TFF2 |
| Staphylococcus warneri E4 | warneriE4 | DCN |
| Klebsiella oxytoca, Strain MIT 10-5250 | NWP53 | PF4V1 |
| Erysipelotrichaceae sp., Strain 6_1_45 | NWP174 | FAM19A3 |
| Bifidobacterium longum subsp. longum, Strain 44B | NWP308 | CCL24 |
| Corynebacterium amycolatum, Strain SK46 | NWP07 | DEFB130A |
| Propionibacterium acidifaciens, Oral Taxon 191, Strain F0233 | NWP263 | ENDOU |
| Lachnospiraceae bacterium, Strain AAB23 | AB23 | SMR3B |
| Enterococcus faecium, Strain TX1330 | NWP19 | SMR3A |
| Staphylococcus hominis B3 | hominisB3 | MUCL3 |
| Streptococcus downei, Oral Taxon 594, Strain F0415 | NWP123 | ASIP |
| Bacteroides sp., Strain D2 | NWP192 | LOC644613 |
| Eggerthella sp., Strain MVA1 | NWP185 | NPPC |
| Arthrobacter albus, Strain DNF00011 | NWP86 | PF4V1 |
| Fusobacterium sp., Oral Taxon 370, Strain F0437 | NWP143 | MUC20 |
| Bifidobacterium sp., Strain MSTE12 | NWP242 | EPHA5 |
| Escherichia coli, Strain MS 124-1 | NWP32 | PF4V1 |
| Staphylococcus saprophyticus E8 | saprophyticusE8 | ADRA2C |
| Streptococcus sp., Strain AS20 | NWP116 | PIANP |
| Escherichia coli, Strain MS 110-3 | NWP35 | AGRP |
| Escherichia coli, Strain 83972 | NWP04 | DEFB130B |
| Erysipelotrichaceae sp., Strain 6_1_45 | NWP174 | PIANP |
| Oscillibacter sp., Strain KLE-1728 | NWP269 | NGFR |
| Staphylococcus pasteuri C9 | pasteuriC9 | CNTN1 |
| Streptococcus gallolyticus subsp. gallolyticus, Strain TX20005 | NWP27 | TMEM119 |
| Proteus mirabilis, Strain WGLW6 | NWP59 | DQA2_Epitope-1 |
| Bacillus sp., Strain DNF00233 | NWP90 | CCL18 |
| Clostridium aldenense, Strain WAL-18727 | NWP314 | PLA2G2E |
| Lactobacillus gasseri, Strain EX33690VC16 | NWP113 | PF4V1 |
| Peptoniphilus lacrimalis, Strain UPII 315-B | NWP261 | PF4V1 |
| Escherichia coli, Strain MS 69-1 | NWP39 | CCL18 |
| Rothia aeria, Oral Taxon 188, Strain F0474 | NWP64 | TMEM119 |
| Lactobacillus gasseri, Strain EX33690VC16 | NWP113 | ENDOU |
| Shigella sp., Strain D9 | NWP06 | PRR27 |
| Fusobacterium nucleatum, Strain CTI-02 | NWP186 | MANF |
| Streptococcus vestibularis, Strain F0396 | NWP132 | CTRB1 |
| Propionibacterium acnes, Strain HL037PA1 | NWP208 | OPRK1 |
| Fusobacterium sp., Strain CM22 | NWP167 | EPHA5 |
| Escherichia coli, Strain MS 21-1 | NWP38 | CEACAM5 |
| Staphylococcus warneri F1 | warneriF1 | MUCL3 |
| Eggerthella sp., Strain MVA1 | NWP185 | FAM19A3 |
| Staphylococcus epidermidis, Strain NIHLM018 | NWP72 | XG |
| Fusobacterium periodonticum, Strain 1_A_54/D10 | NWP142 | MSLN |
| Bacteroides stercoris, Strain CC31F | NWP270 | MCAM |
| Fusobacterium nucleatum, Strain CTI-05 | NWP188 | CCL18 |
| Staphylococcus epidermidis, Strain NIHLM040 | NWP76 | TMEM119 |
| Prevotella buccae, Strain D17 | NWP256 | DPP7 |
| Staphylococcus pasteuri F11 | pasteuriF11 | PODXL2 |
| Streptococcus downei, Oral Taxon 594, Strain F0415 | NWP123 | FGFRL1 |
| Streptococcus anginosus, Strain F0211 | NWP125 | PF4V1 |
| Staphylococcus pasteuri F11 | pasteuriF11 | GYPA |
| Lachnospiraceae sp., Strain DNF00160 | NWP150 | UNQ6190/PRO20217 |
| Staphylococcus warneri, Strain SK66 | NWP11 | CCER2 |
| Actinomyces neuii, Strain MJR8396A | NWP258 | PF4V1 |
| Enterococcus faecium, Strain TX0133a04 | NWP44 | TMEM119 |
| Sporosarcina sp., Strain 2681 | NWP29 | COL10A1 |
| Fusobacterium nucleatum subsp. animalis, Oral Taxon 420, Strain F0419 | NWP196 | SPINK9 |
| Fusobacterium sp., Strain CM21 | NWP166 | PLA2G2E |
| Lactobacillus jensenii, Strain 208-1 | NWP111 | RTBDN |
| Plesiomonas sp., Strain HPP0020 | NWP62 | FAM19A3 |
| Prevotella veroralis, Oral Taxon 572, Strain F0319 | NWP155 | SFN |
| Klebsiella oxytoca, Strain MIT 10-5246 | NWP50 | PF4V1 |
| Staphylococcus pasteuri G7 | pasteuriG7 | KLK13 |
| Staphylococcus epidermidis, Strain BCM0060 | NWP13 | TMEM119 |
| Collinsella sp., Strain 4_8_47FAA | NWP198 | MUCL3 |
| Prevotella timonensis, Strain CRIS 5C-B1 | NWP156 | GPR25 |
| Ruminococcus gnavus, Strain NWP325 | RG74 | TXNDC12 |
| Enterococcus faecium, Strain ERV102 | NWP80 | PIANP |
| Staphylococcus capitis B12 | capitisB12 | CD22 |
| Corynebacterium amycolatum, Strain SK46 | NWP07 | AGRP |
| Eubacterium infirmum, Strain F0142 | NWP223 | PDIA6 |
| Staphylococcus epidermidis, Strain NIHLM070 | NWP69 | PF4V1 |
| Proteus mirabilis, Strain WGLW6 | NWP59 | PSORS1C2 |
| Staphylococcus warneri E4 | warneriE4 | DKK4 |
| Streptococcus sp., Strain AS20 | NWP116 | MUCL3 |
| Propionibacterium acnes, Strain HL037PA1 | NWP208 | TMEM119 |
| Streptococcus sp., Strain AS20 | NWP116 | CTRB2 |
| Micrococcus luteus, Strain SK58 | NWP08 | NPPC |
| Escherichia coli, Strain MS 110-3 | NWP35 | SMR3A |
| Plesiomonas sp., Strain HPP0020 | NWP62 | SMR3A |
| Lactobacillus reuteri, Strain CF48-3A | NWP100 | PF4V1 |
| Staphylococcus saprophyticus E8 | saprophyticusE8 | OSTN |
| Staphylococcus epidermidis C6 | EpiC6 | MUCL3 |
| Streptococcus mitis, Strain F0392 | NWP26 | KLK10 |
| Corynebacterium amycolatum, Strain SK46 | NWP07 | CHRDL2 |
| Lactobacillus jensenii, Strain EX849587VC03 | NWP108 | TMEM119 |
| Propionibacterium acnes, Strain HL025PA1 | NWP204 | FAM174A |
| Enterococcus faecium, Strain ERV165 | NWP81 | PRR27 |
| Staphylococcus warneri E4 | warneriE4 | EPHB3 |
| Staphylococcus epidermidis, Strain NIHLM018 | NWP72 | PF4V1 |
| Klebsiella oxytoca, Strain MIT 10-5246 | NWP50 | TMEM119 |
| Staphylococcus epidermidis, Strain NIHLM020 | NWP74 | PF4V1 |
| Corynebacterium sp., Strain HFH0082 | NWP61 | PRR27 |
| Streptococcus sp., Strain SR1 | NWP122 | UNQ6190/PRO20217 |
| Lactobacillus reuteri, Strain CF48-3A | NWP100 | TMEM119 |
| Propionibacterium acnes, Strain HL002PA1 | NWP206 | OPRK1 |
| Bacteroidales bacterium, Strain KA00344 | NWP302 | IL4 |
| Bacteroides sp., Strain 4_1_36 | NWP312 | FGF1 |
| Streptococcus mitis, Strain F0392 | NWP26 | UNQ6190/PRO20217 |
| Fusobacterium periodonticum, Strain 2_1_31 | NWP160 | ENDOU |
| Streptococcus sp., Strain C150 | NWP121 | UNQ6190/PRO20217 |
| Staphylococcus hominis, Strain C80 | NWP25 | CSAG1 |
| Collinsella aerofaciens, Strain AAB3 | AB3 | ADAMTS16 |
| Propionibacterium acnes, Strain HL013PA1 | NWP209 | OPRK1 |
| Fusobacterium sp., Strain CM22 | NWP167 | TNFRSF4 |
| Corynebacterium amycolatum, Strain SK46 | NWP07 | PRR27 |
| Corynebacterium sp., Strain HFH0082 | NWP61 | DEFB130A |
| Staphylococcus warneri D11 | warneriD12 | PLA2G2A |
| Klebsiella pneumoniae subsp. pneumoniae, Strain WGLW2 | NWP57 | PF4V1 |
| Bacteroides eggerthii, Strain 1_2_48FAA | NWP180 | RCN3 |
| Staphylococcus epidermidis, Strain NIHLM008 | NWP70 | PF4V1 |
| Bacillus sp., Strain DNF00233 | NWP90 | DEFB130B |
| Fusobacterium sp., Strain CM22 | NWP167 | C3AR_Epitope-3 |
| Escherichia sp., Strain 3_2_53FAA | NWP02 | CCL18 |
| Klebsiella pneumoniae subsp. pneumoniae, Strain WGLW1 | NWP56 | PF4V1 |
| Staphylococcus capitis A5 | capitisA5 | PLA2G2E |
| Corynebacterium amycolatum, Strain SK46 | NWP07 | IGFBP5 |
| Propionibacterium acnes, Strain HL044PA1 | NWP237 | TFF1 |
| Staphylococcus epidermidis, Strain NIHLM031 | NWP73 | MUCL3 |
| Staphylococcus warneri D11 | warneriD12 | OPN3 |
| Streptococcus sp., Strain SR1 | NWP122 | PF4V1 |
| Staphylococcus capitis A7 | capitisA7 | EXOC3-AS1 |
| Lachnospiraceae sp., Strain ICM7 | NWP241 | IL29 |
| Lactobacillus jensenii, Strain EX849587VC06 | NWP109 | LOC644613 |
| Lactobacillus jensenii, Strain EX849587VC06 | NWP109 | FAM19A3 |
| Propionibacterium acidifaciens, Oral Taxon 191, Strain F0233 | NWP263 | TFF3 |
| Streptococcus gallolyticus subsp. gallolyticus, Strain TX20005 | NWP27 | PF4V1 |
| Fusobacterium sp., Strain CM22 | NWP153 | RCN2 |
| Klebsiella sp., Strain OBRC7 | NWP60 | PF4V1 |
| Escherichia coli, Strain 83972 | NWP04 | PF4V1 |
| Klebsiella pneumoniae subsp. pneumoniae, Strain WGLW2 | NWP57 | TMEM119 |
| Peptoniphilus sp., Oral Taxon 836, Strain F0141 | NWP313 | IGFBP5 |
| Lactobacillus jensenii, Strain 208-1 | NWP111 | AGER |
| Sporosarcina sp., Strain 2681 | NWP29 | PRR27 |
| Staphylococcus capitis A7 | capitisA7 | ACTR3C |
| Escherichia coli, Strain MS 85-1 | NWP30 | PRR27 |
| Staphylococcus pasteuri C9 | pasteuriC9 | CDSN |
| Escherichia coli, Strain MS 16-3 | NWP37 | LOC644613 |
| Streptococcus sp., Strain SR1 | NWP122 | LGALS3 |
| Lactobacillus crispatus, Strain JV-V01 | NWP107 | CTRB1 |
| Escherichia coli, Strain MS 196-1 | NWP42 | NPPA |
| Lactobacillus gasseri, Strain 224-1 | NWP112 | TMEM119 |
| Enterococcus faecium, Strain ERV165 | NWP81 | MUCL3 |
| Lactobacillus jensenii, Strain EX849587VC03 | NWP108 | PF4V1 |
| Citrobacter freundii, Strain 4_7_47CFAA | NWP88 | CCL21 |
| Lactobacillus jensenii, Strain 269-3 | NWP110 | C3AR_Epitope-3 |
| Escherichia coli, Strain MS 21-1 | NWP38 | TMEM119 |
| Bacteroides fragilis, Strain CL05T00C42 | NWP287 | LCN1 |
| Staphylococcus warneri D9 | warneriD9 | SSBP3-AS1 |
| Escherichia coli, Strain MS 196-1 | NWP42 | PF4V1 |
| Ruminococcus gnavus, Strain AAB28 | AB28 | LOC644613 |
| Staphylococcus warneri D11 | warneriD12 | CNPY3 |
| Fusobacterium sp., Oral Taxon 370, Strain F0437 | NWP143 | GFY_Epitope-1 |
| Staphylococcus aureus, Strain MN8 | NWP17 | CD320 |
| Plesiomonas sp., Strain HPP0020 | NWP62 | AGRP |
| Prevotella disiens, Strain DNF00882 | NWP177 | GLP1R |
| Staphylococcus gallinarum E9 | gallinarumE9 | PDGFB |
| Fusobacterium sp., Strain CM22 | NWP167 | GPRC6A |
| Enterococcus faecalis, Strain TX2137 | NWP117 | LOC644613 |
| Bacteroides stercoris, Strain CC31F | NWP270 | EPHA2 |
| Escherichia coli, Strain MS 115-1 | NWP36 | PRR27 |
| Klebsiella sp., Strain 1_1_55 | NWP03 | PF4V1 |
| Enterococcus faecium, Strain E417 | NWP79 | PF4V1 |
| Ruminococcus gnavus, Strain NWP326 | RG109 | BTC |
| Staphylococcus warneri B9 | warneriB9 | PROCR |
| Bacteroides dorei, Strain 5_1_36/D4 | NWP244 | PVRL4 |
| Streptococcus salivarius, Strain SK126 | NWP118 | MUCL3 |
| Enterococcus faecium, Strain ERV102 | NWP80 | CSAG1 |
| Proteus mirabilis, Strain WGLW6 | NWP59 | ACVR2B |
| Fusobacterium sp., Oral Taxon 370, Strain F0437 | NWP143 | FAM174A |
| Micrococcus luteus, Strain SK58 | NWP08 | FAM19A2 |
| Lactobacillus parafarraginis, Strain F0439 | NWP105 | TFF1 |
| Staphylococcus warneri F1 | warneriF1 | LOC644613 |
| Dorea formicigenerans, Strain AAB36 | AB36 | BTC |
| Staphylococcus capitis, Strain SK14 | NWP09 | PF4V1 |
| Staphylococcus caprae, Strain C87 | NWP24 | UNQ6190/PRO20217 |
| Staphylococcus capitis A7 | capitisA7 | ADRA2C |
| Staphylococcus warneri B9 | warneriB9 | CCER2 |
| Streptococcus mitis, Strain F0392 | NWP26 | SMR3A |
| Staphylococcus xylosus F2 | xylosusF2 | PDGFB |
| Streptococcus downei, Oral Taxon 594, Strain F0415 | NWP123 | CXCL9 |
| Staphylococcus aureus, Strain TCH70 | NWP12 | SHISA7 |
| Proteus mirabilis, Strain WGLW6 | NWP59 | PGA4 |
| Citrobacter freundii, Strain 4_7_47CFAA | NWP28 | CCL5 |
| Catabacter hongkongensis, Strain AB8_9 | NWP299 | COL10A1 |
| Staphylococcus succinus D11 | succinusD11 | ENDOU |
| Enterococcus faecium, Strain E417 | NWP79 | CCL17 |
| Shuttleworthia sp., Strain MSX8B | NWP149 | MILR1 |
| Escherichia coli, Strain MS 196-1 | NWP42 | AGRP |
| Citrobacter sp., Strain 30_2 | NWP01 | PF4V1 |
| Plesiomonas sp., Strain HPP0020 | NWP62 | COL10A1 |
| Enterococcus faecium, Strain E417 | NWP79 | SHISA6 |
| Propionibacterium acnes, Strain HL005PA3 | NWP216 | FAM174A |
| Fusobacterium periodonticum, Strain 2_1_31 | NWP160 | EPHA5 |
| Rhodococcus erythropolis, Strain SK121 | NWP95 | PLA2G2E |
| Staphylococcus pasteuri G7 | pasteuriG7 | PODXL2 |
| Lactobacillus parafarraginis, Strain F0439 | NWP105 | CXCL9 |
| Escherichia coli, Strain MS 119-7 | NWP31 | PF4V1 |
| Veillonella sp., Oral Taxon 780, Strain F0422 | NWP268 | ENDOU |
| Staphylococcus warneri E4 | warneriE4 | PLA2G2A |
| Streptococcus sp., Strain AS20 | NWP116 | FAM19A3 |
| Prevotella histicola, Strain F0411 | NWP144 | S6A17_Epitope-2 |
| Staphylococcus pasteuri G3 | pasteuriG3 | FAM174A |
| Escherichia coli, Strain MS 85-1 | NWP30 | CONA1_Epitope-1 |
| Proteus mirabilis, Strain WGLW6 | NWP59 | TMED1 |
| Propionibacterium acnes, Strain HL050PA1 | NWP248 | OPRK1 |
| Klebsiella oxytoca, Strain MIT 10-5249 | NWP52 | PF4V1 |
| Megasphaera sp., Strain DNF00912 | NWP296 | MFSD2A |
| Eggerthella sp., Strain MVA1 | NWP185 | PIANP |
| Klebsiella oxytoca, Strain MIT 10-5249 | NWP52 | LY6D |
| Bacillus sp., Strain DNF00233 | NWP90 | KLK10 |
| Shigella sp., Strain D9 | NWP06 | LOC644613 |
| Streptococcus sp., Strain OBRC6 | NWP124 | CTRB2 |
| Staphylococcus pasteuri C9 | pasteuriC9 | SOSTDC1 |
| Proteus mirabilis, Strain WGLW6 | NWP59 | OTOS |
| Escherichia coli, Strain 83972 | NWP04 | INSL4 |
| Escherichia coli, Strain MS 115-1 | NWP36 | COL10A1 |
| Fusobacterium nucleatum, Strain CTI-05 | NWP188 | PLA2G2E |
| Enterococcus faecalis, Strain R712 | NWP120 | TMEM119 |
| Propionibacterium acnes, Strain HL060PA1 | NWP239 | CC50C_Epitope-1 |
| Streptococcus anginosus, Strain F0211 | NWP125 | LOC644613 |
| Streptococcus sp., Strain SR1 | NWP122 | RTBDN |
| Catabacter hongkongensis, Strain AB8_9 | NWP299 | EPHA2 |
| Bacteroides stercoris, Strain CC31F | NWP270 | LAYN |
| Bifidobacterium breve, Strain JCP7499 | NWP310 | SPINK9 |
| Fusobacterium nucleatum subsp. animalis, Oral Taxon 420, Strain F0419 | NWP196 | EPHA5 |
| Dorea sp., Strain AAB35 | AB35 | PDIA6 |
| Streptococcus mitis, Strain F0392 | NWP26 | IGFBP5 |
| Citrobacter sp., Strain 30_2 | NWP01 | SPINK9 |
| Propionibacterium acnes, Strain HL025PA1 | NWP204 | TMEM119 |
| Lactobacillus gasseri, Strain JV-V03 | NWP106 | TFF1 |
| Prevotella nigrescens, Strain F0103 | NWP145 | GP6 |
| Propionibacterium acnes, Strain HL002PA1 | NWP206 | TMEM119 |
| Staphylococcus hominis, Strain C80 | NWP25 | UNQ6190/PRO20217 |
| Lachnospiraceae bacterium, Strain AAB23 | AB23 | UNQ9165/PRO28630 |
| Staphylococcus caprae, Strain C87 | NWP24 | TMEM119 |
| Peptoniphilus lacrimalis, Strain UPII 315-B | NWP261 | CAC1D_Epitope-12 |
| Propionibacterium acidifaciens, Oral Taxon 191, Strain F0233 | NWP263 | IGFBP6 |
| Staphylococcus hominis, Strain C80 | NWP25 | CTRB1 |
| Prevotella bivia, Strain DNF00188 | NWP193 | MUCL3 |
| Staphylococcus warneri C2 | warneriC2 | GRIA2_Epitope-2 |
| Sporosarcina sp., Strain 2681 | NWP29 | CONA1_Epitope-1 |
| Staphylococcus epidermidis, Strain NIHLM015 | NWP71 | TMEM119 |
| Enterococcus faecium, Strain E417 | NWP79 | PIANP |
| Staphylococcus epidermidis, Strain NIHLM031 | NWP73 | PF4V1 |
| Escherichia sp., Strain 3_2_53FAA | NWP02 | PLA2G2E |
| Lactobacillus crispatus, Strain GED7749C | NWP104 | MCHR1 |
| Escherichia coli, Strain MS 110-3 | NWP35 | PRR27 |
| Staphylococcus warneri D11 | warneriD12 | EXOC3-AS1 |
| Staphylococcus warneri E6 | warneriE6 | CRELD1 |
| Bacteroidales bacterium, Strain KA00344 | NWP302 | C9orf47 |
| Proteus mirabilis, Strain WGLW6 | NWP59 | LRRN4CL |
| Catabacter hongkongensis, Strain AB8_9 | NWP299 | PLA2G2E |
| Plesiomonas sp., Strain HPP0020 | NWP62 | PIANP |
| Staphylococcus epidermidis, Strain NIHLM003 | NWP67 | PF4V1 |
| Enterococcus faecalis, Strain R712 | NWP120 | PF4V1 |
| Staphylococcus caprae, Strain C87 | NWP24 | CTRB1 |
| Erysipelotrichaceae sp., Strain 6_1_45 | NWP174 | COL10A1 |
| Propionibacterium acnes, Strain HL013PA1 | NWP209 | FAM174A |
| Streptococcus downei, Oral Taxon 594, Strain F0415 | NWP123 | CCL17 |
| Escherichia coli, Strain MS 200-1 | NWP41 | NPPA |
| Bacteroides stercoris, Strain CC31F | NWP270 | CD97_1_2_5 |
| Escherichia coli, Strain MS 69-1 | NWP39 | SMR3A |
| Clostridium sp., Strain MSTE9 | NWP253 | COL10A1 |
| Proteus mirabilis, Strain WGLW6 | NWP59 | CD320 |
| Lactobacillus jensenii, Strain 269-3 | NWP110 | MTLR_Epitope-3 |
| Plesiomonas sp., Strain HPP0020 | NWP62 | DEFB130B |
| Staphylococcus aureus, Strain TCH70 | NWP12 | C1QL3 |
| Lactobacillus parafarraginis, Strain F0439 | NWP105 | CCL17 |
| Staphylococcus haemolyticus B5 | haemolyticusB5 | KCNK1 |
| Sporosarcina sp., Strain 2681 | NWP29 | MUC7 |
| Enterococcus faecalis, Strain S613 | NWP119 | PF4V1 |
| Fusobacterium nucleatum, Strain CTI-02 | NWP186 | PLA2G2E |
| Oscillibacter sp., Strain KLE 1745 | NWP252 | PDIA2 |
| Escherichia coli, Strain MS 85-1 | NWP30 | AGRP |
| Staphylococcus capitis B12 | capitisB12 | CCER2 |
| Erysipelotrichaceae sp., Strain 6_1_45 | NWP174 | CSAG1 |
| Propionibacterium acnes, Strain HL050PA3 | NWP249 | OPRK1 |
| Staphylococcus warneri E6 | warneriE6 | EPHA6 |
| Fusobacterium sp., Strain CM21 | NWP166 | MUCL3 |
| Arthrobacter albus, Strain DNF00011 | NWP86 | CTRB1 |
| Eubacterium infirmum, Strain F0142 | NWP223 | SPINK9 |
| Enterococcus faecium, Strain E417 | NWP79 | CXCL9 |
| Weissella cibaria, Strain F16_1 | NWP101 | LOC644613 |
| Bacteroides vulgatus, Strain CL09T03C04 | NWP283 | EPHA2 |
| Escherichia coli, Strain MS 115-1 | NWP36 | PRND |
| Staphylococcus epidermidis, Strain NIHLM040 | NWP76 | C2orf40 |
| Staphylococcus warneri B9 | warneriB9 | BMPR1B |
| Escherichia coli, Strain MS 115-1 | NWP36 | SEMG1 |
| Staphylococcus epidermidis, Strain NIHLM001 | NWP66 | PIANP |
| Staphylococcus saprophyticus E8 | saprophyticusE8 | CCL25 |
| Plesiomonas sp., Strain HPP0020 | NWP62 | PRR27 |
| Lactobacillus parafarraginis, Strain F0439 | NWP105 | PIANP |
| Clostridium difficile, Strain 050-P50-2011 | NWP278 | ENDOU |
| Fusobacterium nucleatum, Strain CTI-07 | NWP190 | FAM19A2 |
| Streptococcus mitis, Strain F0392 | NWP26 | FGF17 |
| Corynebacterium amycolatum, Strain SK46 | NWP07 | FGF19 |
| Lactobacillus jensenii, Strain 208-1 | NWP111 | FGF17 |
| Bacteroides stercoris, Strain CC31F | NWP270 | PF4V1 |
| Enterococcus faecium, Strain E417 | NWP79 | RTBDN |
| Staphylococcus warneri D4 | warneriD4 | C2orf40 |
| Enterococcus faecium, Strain TX0133a04 | NWP44 | ENDOU |
| Streptococcus parasanguinis, Strain F0449 | NWP114 | PF4V1 |
| Propionibacterium acnes, Strain HL053PA2 | NWP236 | TMEM119 |
| Shigella sp., Strain D9 | NWP06 | PIANP |
| Staphylococcus capitis C4 | capitisC4 | LOC644613 |
| Finegoldia magna, Strain GED7760A | NWP200 | PDIA6 |
| Enterococcus faecium, Strain E417 | NWP79 | PGLYRP1 |
| Lachnospiraceae bacterium, Strain AAB19 | AB19 | CSN3 |
| Staphylococcus succinus D11 | succinusD11 | PDGFB |
| Staphylococcus warneri F1 | warneriF1 | CSPG5 |
| Prevotella bivia, Strain DNF00650 | NWP176 | IL22 |
| Proteus mirabilis, Strain WGLW4 | NWP58 | PF4V1 |
| Proteus mirabilis, Strain WGLW6 | NWP59 | GHRHR |
| Streptococcus sp., Strain C150 | NWP121 | CTRB1 |
| Arthrobacter albus, Strain DNF00011 | NWP86 | CCL2 |
| Bacteroides dorei, Strain 5_1_36/D4 | NWP244 | SPINK9 |
| Lactobacillus crispatus, Strain GED7749C | NWP104 | LILRA3 |
| Proteus mirabilis, Strain WGLW6 | NWP59 | CGREF1 |
| Lactobacillus reuteri, Strain CF48-3A | NWP100 | TFF3 |
| Prevotella sp., Oral Taxon 317, Strain F0108 | NWP173 | PSORS1C2 |
| Klebsiella oxytoca, Strain MIT 10-5242 | NWP46 | SHISA2 |
| Streptococcus anginosus, Strain F0211 | NWP125 | RTBDN |
| Escherichia coli, Strain MS 69-1 | NWP39 | COL10A1 |
| Fusobacterium sp., Oral Taxon 370, Strain F0437 | NWP143 | C3AR_Epitope-3 |
| Streptococcus sp., Strain SR1 | NWP122 | PDGFB |
| Propionibacterium acnes, Strain HL025PA2 | NWP210 | TMEM119 |
| Citrobacter freundii, Strain 4_7_47CFAA | NWP28 | SPINK9 |
| Lactobacillus jensenii, Strain EX849587VC06 | NWP109 | AVP |
| Citrobacter sp., Strain 30_2 | NWP01 | GNLY |
| Staphylococcus warneri E4 | warneriE4 | OPN3 |
| Staphylococcus capitis A7 | capitisA7 | ECE1 |
| Bacteroides stercoris, Strain CC31F | NWP270 | BSG |
| Streptococcus sp., Strain CM7 | NWP133 | UNQ6190/PRO20217 |
| Erysipelotrichaceae sp., Strain 6_1_45 | NWP174 | SMR3A |
| Bacteroides fragilis, Strain CL05T00C42 | NWP287 | AZGP1 |
| Corynebacterium sp., Strain HFH0082 | NWP61 | IGFBP5 |
| Propionibacterium acnes, Strain HL025PA2 | NWP210 | SPINT3 |
| Collinsella aerofaciens, Strain AAB3 | AB3 | PLA2G2E |
| Streptococcus sp., Strain BS29a | NWP129 | PDGFB |
| Prevotella veroralis, Oral Taxon 572, Strain F0319 | NWP155 | MUCL3 |
| Collinsella aerofaciens, Strain AAB3 | AB3 | CSN3 |
| Enterococcus faecium, Strain TX1330 | NWP19 | C4A |
| Staphylococcus capitis B8 | capitisB8 | FAM19A3 |
| Enterococcus faecium, Strain TX1330 | NWP19 | PRR27 |
| Prevotella bivia, Strain GED7760C | NWP233 | FGF17 |
| Escherichia coli, Strain MS 200-1 | NWP41 | DEFB130A |
| Prevotella disiens, Strain DNF00882 | NWP177 | GPER1_Epitope-1 |
| Staphylococcus epidermidis, Strain NIHLM031 | NWP73 | CTRB2 |
| Eubacterium sp., Strain AS15 | NWP254 | COL10A1 |
| Eubacterium infirmum, Strain F0142 | NWP223 | ENDOU |
| Streptococcus salivarius, Strain SK126 | NWP118 | FAM19A3 |
| Streptococcus sp., Strain BS21 | NWP131 | FAM168B |
| Sporosarcina sp., Strain 2681 | NWP29 | SEMG1 |
| Lactobacillus jensenii, Strain 208-1 | NWP111 | PIANP |
| Staphylococcus aureus, Strain MN8 | NWP17 | KCNK1 |
| Enterococcus faecium, Strain TX1330 | NWP19 | SEMG1 |
| Streptococcus parasanguinis, Strain F0449 | NWP114 | FZD8 |
| Sporosarcina sp., Strain 2681 | NWP29 | DRAXIN |
| Megasphaera sp., Strain DNF00912 | NWP296 | PLA2G2E |
| Enterococcus faecalis, Strain TX2137 | NWP117 | TMEM119 |
| Streptococcus sp., Strain AS20 | NWP116 | DEFB130B |
| Streptococcus sp., Strain SR4 | NWP130 | PIANP |
| Blautia sp., Strain AAB30 | AB30 | SMR3A |
| Staphylococcus epidermidis, Strain NIH04008 | NWP77 | CSAG1 |
| Micrococcus luteus, Strain SK58 | NWP08 | DEFB130A |
| Sporosarcina sp., Strain 2681 | NWP29 | RCN1 |
| Lactobacillus jensenii, Strain EX849587VC03 | NWP108 | DEFB130B |
| Lactobacillus jensenii, Strain EX849587VC06 | NWP109 | DEFB130A |
| Blautia sp., Strain AAB30 | AB30 | MUC7 |
| Staphylococcus capitis B12 | capitisB12 | CLCC1 |
| Staphylococcus capitis A7 | capitisA7 | PLA2G2A |
| Staphylococcus capitis E7 | capitisE7 | FAM19A3 |
| Finegoldia magna, Strain SY01 | NWP138 | GPR25 |
| Ruminococcus gnavus, Strain NWP325 | RG74 | MFSD2A |
| Staphylococcus capitis C4 | capitisC4 | FAM19A3 |
| Staphylococcus warneri E4 | warneriE4 | CDSN |
| Corynebacterium amycolatum, Strain SK46 | NWP07 | LECT2 |
| Propionibacterium acnes, Strain HL050PA1 | NWP248 | TMEM119 |
| Escherichia coli, Strain MS 16-3 | NWP37 | CCL18 |
| Lachnospiraceae bacterium, Strain AAB23 | AB23 | INSL4 |
| Prevotella nigrescens, Strain F0103 | NWP145 | CLDN7 |
| Escherichia coli, Strain MS 16-3 | NWP37 | PIANP |
| Staphylococcus haemolytics, Strain DNF00585 | NWP87 | PF4V1 |
| Lactobacillus jensenii, Strain EX849587VC06 | NWP109 | FGF17 |
| Arthrobacter albus, Strain DNF00011 | NWP86 | AVP |
| Eubacterium sp., Strain 3_1_31 | NWP182 | LOC644613 |
| Staphylococcus warneri C2 | warneriC2 | AMELX |
| Collinsella sp., Strain 4_8_47FAA | NWP198 | LOC644613 |
| Lactobacillus jensenii, Strain EX849587VC03 | NWP108 | FAM19A3 |
| Rothia aeria, Oral Taxon 188, Strain F0474 | NWP64 | PF4V1 |
| Staphylococcus saprophyticus G1 | saprophyticusG1 | ENDOU |
| Enterococcus faecium, Strain ERV102 | NWP80 | PLA2G2E |
| Citrobacter freundii, Strain 4_7_47CFAA | NWP88 | PF4V1 |
| Escherichia coli, Strain MS 200-1 | NWP41 | FAM19A3 |
| Fusobacterium nucleatum, Strain CTI-05 | NWP188 | PRR27 |
| Corynebacterium sp., Strain HFH0082 | NWP61 | AGRP |
| Fusobacterium sp., Oral Taxon 370, Strain F0437 | NWP143 | SMR3B |
| Fusobacterium nucleatum, Strain CTI-05 | NWP188 | PIANP |
| Enterococcus faecium, Strain E417 | NWP79 | CTRB1 |
| Staphylococcus warneri, Strain SK66 | NWP11 | OSTN |
| Staphylococcus capitis B12 | capitisB12 | CALU |
| Bacteroides sp., Strain 4_1_36 | NWP312 | IGFBP6 |
| Streptococcus sobrinus, Strain W1703 | NWP85 | FAM19A3 |
| Fusobacterium sp., Strain CM21 | NWP166 | CCL18 |
| Staphylococcus haemolyticus B6 | haemolyticusB6 | KCNK1 |
| Bacteroides stercoris, Strain CC31F | NWP270 | C19orf10 |
| Bacteroides fragilis, Strain 3_1_12 | NWP271 | EPHA5 |
| Erysipelotrichaceae sp., Strain 6_1_45 | NWP181 | IGFBP5 |
| Corynebacterium sp., Strain HFH0082 | NWP61 | CHRDL2 |
| Escherichia coli, Strain MS 69-1 | NWP39 | NPPC |
| Lactobacillus gasseri, Strain JV-V03 | NWP106 | PEBP4 |
| Staphylococcus capitis A7 | capitisA7 | CSF2RA |
| Escherichia coli, Strain 83972 | NWP04 | SMR3A |
| Weissella cibaria, Strain F16_1 | NWP101 | MUCL3 |
| Escherichia sp., Strain 3_2_53FAA | NWP02 | FAM19A3 |
| Lactobacillus jensenii, Strain 208-1 | NWP111 | FAM19A3 |
| Escherichia coli, Strain MS 115-1 | NWP36 | SMR3A |
| Staphylococcus capitis A7 | capitisA7 | PLA2G3 |
| Sporosarcina sp., Strain 2681 | NWP29 | AGRP |
| Enterococcus faecium, Strain E417 | NWP79 | PEBP4 |
| Erysipelotrichaceae sp., Strain 6_1_45 | NWP174 | DEFB130B |
| Escherichia coli, Strain MS 115-1 | NWP36 | AGRP |
| Prevotella melaninogenica, Strain D18 | NWP151 | TXNDC12 |
| Staphylococcus intermedius F7 | intermediusF7 | PLA2G2E |
| Bacteroides stercoris, Strain CC31F | NWP270 | KCNK1 |
| Corynebacterium amycolatum, Strain SK46 | NWP07 | PRND |
| Staphylococcus hominis B3 | hominisB3 | BTC |
| Streptococcus sp., Strain OBRC6 | NWP124 | PIANP |
| Lactobacillus jensenii, Strain 208-1 | NWP111 | FGFRL1 |
| Propionibacterium acnes, Strain HL059PA1 | NWP250 | OPRK1 |
| Streptococcus sp., Strain OBRC6 | NWP124 | FGF17 |
| Streptococcus mitis, Strain F0392 | NWP26 | CHRDL2 |
| Enterococcus faecalis, Strain TX2137 | NWP117 | UNQ6190/PRO20217 |
| Escherichia sp., Strain 3_2_53FAA | NWP02 | TMEM119 |
| Staphylococcus capitis A7 | capitisA7 | EPHB3 |
| Lachnospiraceae bacterium, Strain AAB23 | AB23 | COL10A1 |
| Enterococcus faecalis, Strain TUSoD Ef11 | NWP05 | TMEM119 |
| Bacteroides sp., Strain D2 | NWP192 | PIANP |
| Staphylococcus pasteuri C9 | pasteuriC9 | NLGN3 |
| Enterococcus faecium, Strain ERV102 | NWP80 | CCL18 |
| Fusobacterium nucleatum, Strain CTI-07 | NWP190 | DRAXIN |
| Bacteroides sp., Strain 4_1_36 | NWP312 | SPINK9 |
| Lactobacillus gasseri, Strain JV-V03 | NWP106 | CTRB2 |
| Corynebacterium amycolatum, Strain SK46 | NWP07 | MUC7 |
| Staphylococcus capitis, Strain SK14 | NWP09 | TMEM119 |
| Proteus mirabilis, Strain WGLW6 | NWP59 | SERPINI1 |
| Escherichia coli, Strain MS 110-3 | NWP35 | SEMG1 |
| Citrobacter freundii, Strain 4_7_47CFAA | NWP28 | AGER |
| Staphylococcus warneri D11 | warneriD12 | CRELD1 |
| Fusobacterium sp., Strain CM21 | NWP166 | COL10A1 |
| Staphylococcus warneri E6 | warneriE6 | CDSN |
| Staphylococcus epidermidis B11 | EpiB11 | XG |
| Staphylococcus capitis E12 | capitisE12 | MUCL3 |
| Staphylococcus cohnii G11 | cohniiG11 | KCNK4_Epitope-1 |
| Staphylococcus aureus, Strain CM05 | NWP83 | SO1C1_Epitope-5 |
| Bifidobacterium longum subsp. longum, Strain 44B | NWP308 | FAM19A3 |
| Streptococcus mitis, Strain F0392 | NWP26 | COL10A1 |
| Staphylococcus capitis C11 | capitisC11 | AMELX |
| Escherichia coli, Strain MS 110-3 | NWP35 | FAM19A2 |
| Escherichia coli, Strain MS 85-1 | NWP30 | SEMG1 |
| Bacteroidales bacterium, Strain KA00344 | NWP302 | PANX3 |
| Enterococcus faecium, Strain ERV165 | NWP81 | FGF17 |
| Staphylococcus pasteuri C9 | pasteuriC9 | IGFBP3 |
| Peptoniphilus sp., Oral Taxon 836, Strain F0141 | NWP313 | SMR3A |
| Enterococcus faecalis, Strain S613 | NWP119 | TMEM119 |
| Shigella sp., Strain D9 | NWP06 | PF4V1 |
| Bacteroides sp., Strain D2 | NWP192 | INSL3 |
| Enterococcus faecium, Strain E417 | NWP79 | FGFRL1 |
| Staphylococcus saprophyticus E2 | saprophyticusE2 | MUCL3 |
| Escherichia coli, Strain MS 16-3 | NWP37 | FAM19A3 |
| Citrobacter freundii, Strain 4_7_47CFAA | NWP28 | GNLY |
| Enterococcus faecium, Strain TX1330 | NWP19 | FAM19A2 |
| Sporosarcina sp., Strain 2681 | NWP29 | KLK10 |
| Staphylococcus epidermidis, Strain NIH04008 | NWP77 | RTBDN |
| Staphylococcus warneri E6 | warneriE6 | OR1L1_Epitope-1 |
| Staphylococcus cohnii G11 | cohniiG11 | SCGB1A1 |
| Escherichia coli, Strain MS 69-1 | NWP39 | DEFB130A |
| Staphylococcus epidermidis, Strain M23864:W2 | NWP16 | XG |
| Staphylococcus pasteuri E1 | pasteuriE1 | CSF2RA |
| Staphylococcus pasteuri C9 | pasteuriC9 | TMEM8 |
| Citrobacter freundii, Strain 4_7_47CFAA | NWP28 | PF4V1 |
| Staphylococcus pasteuri C9 | pasteuriC9 | RLN2 |
| Arthrobacter albus, Strain DNF00011 | NWP86 | CAC1F_Epitope-6 |
| Prevotella nigrescens, Strain F0103 | NWP145 | PDIA6 |
| Propionibacterium acnes, Strain HL037PA2 | NWP205 | NLGN4X |
| Enterococcus faecium, Strain TX1330 | NWP19 | FGF17 |
| Propionibacterium acnes, Strain SK182 | NWP306 | FAM174A |
| Bacteroides sp., Strain D2 | NWP192 | FAM19A3 |
| Lactobacillus parafarraginis, Strain F0439 | NWP105 | FGFRL1 |
| Micrococcus luteus, Strain SK58 | NWP08 | COL10A1 |
| Corynebacterium amycolatum, Strain SK46 | NWP07 | SEMG1 |
| Staphylococcus warneri E6 | warneriE6 | EPO |
| Klebsiella oxytoca, Strain MIT 09-7231 | NWP45 | CHGA |
| Megasphaera micronuciformis, Strain DNF00954 | NWP307 | ENDOU |
| Staphylococcus capitis E12 | capitisE12 | LOC644613 |
| Escherichia coli, Strain MS 196-1 | NWP42 | NPPC |
| Staphylococcus capitis B12 | capitisB12 | CLU |
| Fusobacterium sp., Strain CM22 | NWP167 | BTNL8 |
| Fusobacterium sp., Strain CM21 | NWP166 | NPPC |
| Peptoniphilus sp., Strain CMW7756A | NWP162 | MFSD8_Epitope-5 |
| Fusobacterium nucleatum subsp. animalis, Oral Taxon 420, Strain F0419 | NWP196 | C1QTNF2 |
| Lactobacillus parafarraginis, Strain F0439 | NWP105 | BTC |
| Staphylococcus saprophyticus E8 | saprophyticusE8 | CSPG5 |
| Enterococcus faecium, Strain TX1330 | NWP19 | FGF19 |
| Staphylococcus warneri B9 | warneriB9 | TFPI2 |
| Staphylococcus warneri, Strain SK66 | NWP11 | NPY5R |
| Klebsiella oxytoca, Strain MIT 10-5242 | NWP46 | PDCD1 |
| Streptococcus salivarius, Strain SK126 | NWP118 | PEBP4 |
| Peptoniphilus sp., Oral Taxon 836, Strain F0141 | NWP313 | COL10A1 |
| Blautia sp., Strain AAB30 | AB30 | ADAMTS16 |
| Klebsiella pneumoniae subsp. pneumoniae, Strain WGLW1 | NWP56 | FAM19A3 |
| Escherichia coli, Strain MS 85-1 | NWP30 | COL10A1 |
| Eubacterium sp., Strain AS15 | NWP254 | PF4V1 |
| Propionibacterium acnes, Strain HL030PA2 | NWP207 | TMEM119 |
| Staphylococcus aureus, Strain TCH70 | NWP12 | NRN1L |
| Streptococcus sp., Strain CM7 | NWP133 | CTRB1 |
| Staphylococcus warneri E4 | warneriE4 | PLA2G3 |
| Propionibacterium acnes, Strain HL037PA1 | NWP208 | SPINT3 |
| Bacteroides fragilis, Strain 3_1_12 | NWP271 | LGALS7 |
| Escherichia coli, Strain MS 200-1 | NWP41 | NPPC |
| Citrobacter freundii, Strain 4_7_47CFAA | NWP28 | ASIP |
| Ruminococcus gnavus, Strain NWP326 | RG109 | CTRB2 |
| Prevotella disiens, Strain DNF00882 | NWP177 | EPHA5 |
| Lactobacillus crispatus, Strain JV-V01 | NWP107 | ENDOU |
| Enterococcus faecium, Strain TX1330 | NWP19 | AGRP |
| Lactobacillus parafarraginis, Strain F0439 | NWP105 | PEBP4 |
| Propionibacterium acnes, Strain HL005PA4 | NWP220 | CST6 |
| Staphylococcus warneri E6 | warneriE6 | ECE1 |
| Arthrobacter albus, Strain DNF00011 | NWP86 | TMEM119 |
| Staphylococcus saprophyticus E2 | saprophyticusE2 | LOC644613 |
| Staphylococcus capitis B8 | capitisB8 | SMR3A |
| Propionibacterium propionicum, Oral Taxon 739, Strain F0230 | NWP301 | AVP |
| Staphylococcus cohnii G11 | cohniiG11 | KCNK1 |
| Lactobacillus gasseri, Strain EX33690VC16 | NWP113 | CTRB2 |
| Klebsiella pneumoniae subsp. pneumoniae, Strain WGLW3 | NWP54 | PF4V1 |
| Lactobacillus gasseri, Strain JV-V03 | NWP106 | FAM19A3 |
| Lactobacillus jensenii, Strain 208-1 | NWP111 | CXCL9 |
| Micrococcus luteus, Strain SK58 | NWP08 | LECT2 |
| Fusobacterium sp., Oral Taxon 370, Strain F0437 | NWP143 | CD99L2 |
| Fusobacterium nucleatum, Strain CTI-07 | NWP190 | SOST |
| Streptococcus sp., Strain ACS2 | NWP115 | TMEM119 |
| Enterococcus faecium, Strain ERV165 | NWP81 | SEMG1 |
| Streptococcus salivarius, Strain SK126 | NWP118 | UNQ6190/PRO20217 |
| Enterococcus faecium, Strain TX1330 | NWP19 | IGFBP5 |
| Capnocytophaga sp., Oral Taxon 336, Strain F0502 | NWP139 | PDIA3 |
| Staphylococcus saprophyticus D6 | saprophyticusD6 | LOC644613 |
| Blautia sp., Strain KLE 1732 | NWP292 | SMR3A |
| Staphylococcus capitis E7 | capitisE7 | COL10A1 |
| Escherichia sp., Strain 3_2_53FAA | NWP02 | PIANP |
| Staphylococcus epidermidis, Strain NIHLM001 | NWP66 | FAM19A3 |
| Micrococcus luteus, Strain SK58 | NWP08 | IGFBP5 |
| Streptococcus gallolyticus subsp. gallolyticus, Strain TX20005 | NWP27 | DEFA6 |
| Catabacter hongkongensis, Strain AB8_9 | NWP299 | IL1A |
| Prevotella histicola, Strain F0411 | NWP144 | IL1F9 |
| Propionibacterium acnes, Strain HL059PA1 | NWP250 | FAM174A |
| Corynebacterium sp., Strain HFH0082 | NWP61 | C4A |
| Blautia sp., Strain KLE 1732 | NWP292 | NPPC |
| Staphylococcus capitis B12 | capitisB12 | CXCR3 |
| Eggerthella sp., Strain MVA1 | NWP185 | CONA1_Epitope-1 |
| Streptococcus sp., Strain AS20 | NWP116 | FGF17 |
| Staphylococcus capitis B12 | capitisB12 | DRAXIN |
| Staphylococcus epidermidis, Strain NIHLM001 | NWP66 | RTBDN |
| Propionibacterium acnes, Strain HL013PA2 | NWP217 | SLC22A31 |
| Streptococcus gallolyticus subsp. gallolyticus, Strain TX20005 | NWP27 | TMEM149 |
| Staphylococcus caprae, Strain C87 | NWP24 | PIANP |
| Micrococcus luteus, Strain SK58 | NWP08 | FGF17 |
| Staphylococcus aureus, Strain MN8 | NWP17 | MSMB |
| Staphylococcus warneri D11 | warneriD12 | SFRP4 |
| Bacteroides sp., Strain 1_1_30 | NWP272 | ENDOU |
| Staphylococcus capitis E12 | capitisE12 | ENDOU |
| Clostridium difficile, Strain 002-P50-2011 | NWP230 | PDGFB |
| Streptococcus sp., Strain ACC21 | NWP134 | UNQ6190/PRO20217 |
| Escherichia coli, Strain MS 200-1 | NWP41 | PIANP |
| Staphylococcus warneri D11 | warneriD12 | EPO |
| Streptococcus mitis, Strain F0392 | NWP26 | PRR27 |
| Collinsella aerofaciens, Strain AAB3 | AB3 | NPPA |
| Dorea sp., Strain AAB35 | AB35 | SLC8B1 |
| Shigella sp., Strain D9 | NWP06 | DEFB130B |
| Enterococcus faecium, Strain ERV102 | NWP80 | PGLYRP1 |
| Staphylococcus capitis A7 | capitisA7 | CRELD1 |
| Enterococcus faecium, Strain ERV165 | NWP81 | COL10A1 |
| Lactobacillus jensenii, Strain 269-3 | NWP110 | PF4V1 |
| Staphylococcus warneri E4 | warneriE4 | RTN4R |
| Klebsiella pneumoniae subsp. pneumoniae, Strain WGLW1 | NWP56 | CCL18 |
| Staphylococcus warneri E4 | warneriE4 | CNPY3 |
| Enterococcus faecium, Strain TX1330 | NWP19 | CHRDL2 |
| Streptococcus sp., Strain BS21 | NWP131 | PDGFB |
| Escherichia coli, Strain MS 145-7 | NWP33 | ALPP |
| Klebsiella oxytoca, Strain MIT 10-5249 | NWP52 | CLU |
| Streptococcus anginosus, Strain F0211 | NWP125 | BTC |
| Erysipelotrichaceae sp., Strain 6_1_45 | NWP181 | DEFB130A |
| Enterococcus faecalis, Strain R712 | NWP120 | SCNAA_Epitope-3 |
| Staphylococcus pasteuri E1 | pasteuriE1 | KLK13 |
| Klebsiella oxytoca, Strain MIT 10-5246 | NWP50 | AVP |
| Staphylococcus pasteuri F12 | pasteuriF12 | PODXL2 |
| Proteus mirabilis, Strain WGLW6 | NWP59 | TMEFF2 |
| Staphylococcus warneri D11 | warneriD12 | ECE1 |
| Klebsiella pneumoniae subsp. pneumoniae, Strain WGLW3 | NWP54 | LOC644613 |
| Propionibacterium acnes, Strain HL050PA1 | NWP248 | FAM174A |
| Streptococcus sp., Strain BS29a | NWP129 | MUCL3 |
| Escherichia coli, Strain MS 145-7 | NWP33 | LGALS7 |
| Eubacterium infirmum, Strain F0142 | NWP223 | CLPSL1 |
| Staphylococcus epidermidis, Strain E13A | NWP63 | TMEM119 |
| Enterococcus faecium, Strain E417 | NWP79 | CTRB2 |
| Escherichia coli, Strain MS 16-3 | NWP37 | DEFB130B |
| Staphylococcus saprophyticus D10 | saprophyticusD10 | MUCL3 |
| Sporosarcina sp., Strain 2681 | NWP29 | C4A |
| Staphylococcus saprophyticus E8 | saprophyticusE8 | GABRB2 |
| Fusobacterium sp., Strain CM22 | NWP167 | HCTR1 |
| Lachnospiraceae sp., Strain DNF00160 | NWP150 | CTRB1 |
| Fusobacterium sp., Oral Taxon 370, Strain F0437 | NWP143 | LAIR1 |
| Staphylococcus warneri E6 | warneriE6 | GPHB5 |
| Bacteroides sp., Strain D2 | NWP192 | IGFBP5 |
| Enterococcus faecium, Strain ERV102 | NWP80 | NPPC |
| Staphylococcus warneri D11 | warneriD12 | RTN4R |
| Staphylococcus capitis B8 | capitisB8 | LOC644613 |
| Staphylococcus epidermidis H4 | EpiH4 | MUCL3 |
| Staphylococcus epidermidis, Strain NIHLM003 | NWP67 | TMEM119 |
| Streptococcus cristatus, Strain F0329 | NWP126 | AXL |
| Paenisporosarcina sp., Strain HGH0030 | NWP94 | PLA2G2E |
| Fusobacterium periodonticum, Strain 1_A_54/D10 | NWP142 | EPHA5 |
| Proteus mirabilis, Strain WGLW6 | NWP59 | IFNA17 |
| Escherichia sp., Strain 3_2_53FAA | NWP02 | PF4V1 |
| Corynebacterium sp., Strain HFH0082 | NWP61 | KLK10 |
| Streptococcus salivarius, Strain SK126 | NWP118 | FGF17 |
| Collinsella aerofaciens, Strain AAB3 | AB3 | AGRP |
| Escherichia coli, Strain MS 110-3 | NWP35 | KLK10 |
| Blautia sp., Strain AAB32 | AB32 | TFF3 |
| Escherichia coli, Strain MS 200-1 | NWP41 | CCL18 |
| Fusobacterium nucleatum subsp. animalis, Oral Taxon 420, Strain F0419 | NWP196 | SERPINE1 |
| Staphylococcus aureus, Strain TCH70 | NWP12 | SO1C1_Epitope-5 |
| Staphylococcus epidermidis G10 | EpiG10 | PDIA2 |
| Bacteroides stercoris, Strain CC31F | NWP270 | CNPY2 |
| Propionibacterium acnes, Strain HL056PA1 | NWP267 | CNGB3_Epitope-3 |
| Shuttleworthia sp., Strain MSX8B | NWP149 | MUCL3 |
| Propionibacterium acnes, Strain HL027PA1 | NWP211 | DSA2D_Epitope-1 |
| Collinsella aerofaciens, Strain AAB3 | AB3 | LOC644613 |
| Erysipelotrichaceae sp., Strain 6_1_45 | NWP174 | IGFBP5 |
| Bacteroides sp., Strain D2 | NWP192 | CHRDL2 |
| Staphylococcus warneri E4 | warneriE4 | EXOC3-AS1 |
| Staphylococcus capitis E7 | capitisE7 | LOC644613 |
| Enterococcus faecium, Strain ERV165 | NWP81 | UNQ6190/PRO20217 |
| Escherichia coli, Strain MS 110-3 | NWP35 | FGF17 |
| Staphylococcus warneri B9 | warneriB9 | MMP24 |
| Prevotella bivia, Strain GED7760C | NWP233 | TMEM119 |
| Lachnospiraceae bacterium, Strain AAB19 | AB19 | AMY2B |
| Enterococcus faecium, Strain ERV102 | NWP80 | PF4V1 |
| Propionibacterium acnes, Strain HL005PA4 | NWP220 | TMEM119 |
| Escherichia coli, Strain MS 16-3 | NWP37 | COL10A1 |
| Escherichia coli, Strain MS 110-3 | NWP35 | IGFBP5 |
| Prevotella sp., Oral Taxon 317, Strain F0108 | NWP173 | SDC1 |
| Propionibacterium acnes, Strain HL059PA1 | NWP250 | TMEM119 |
| Propionibacterium acnes, Strain HL005PA1 | NWP224 | CNGB3_Epitope-3 |
| Lactobacillus crispatus, Strain JV-V01 | NWP107 | CTRB2 |
| Enterococcus faecium, Strain ERV102 | NWP80 | CTRB1 |
| Dorea formicigenerans, Strain AAB36 | AB36 | FAM19A4 |
| Escherichia coli, Strain MS 200-1 | NWP41 | SMR3A |
| Escherichia coli, Strain MS 16-3 | NWP37 | SMR3A |
| Fusobacterium nucleatum, Strain CTI-07 | NWP190 | CCL18 |
| Staphylococcus epidermidis, Strain NIHLM020 | NWP74 | CTRB2 |
| Staphylococcus pasteuri G3 | pasteuriG3 | TNFRSF21 |
| Streptococcus sobrinus, Strain W1703 | NWP85 | LYVE1 |
| Staphylococcus pasteuri G7 | pasteuriG7 | FAM174A |
| Escherichia coli, Strain 83972 | NWP04 | COL10A1 |
| Escherichia coli, Strain MS 69-1 | NWP39 | SEMG1 |
| Proteus mirabilis, Strain WGLW6 | NWP59 | EDAR |
| Rothia mucilaginosa, Strain CC87LB | NWP84 | MUCL3 |
| Escherichia coli, Strain MS 200-1 | NWP41 | PLA2G2E |
| Streptococcus sp., Strain BS29a | NWP129 | ENDOU |
| Lactobacillus jensenii, Strain EX849587VC06 | NWP109 | CLU |
| Staphylococcus warneri B9 | warneriB9 | PDGFA |
| Enterococcus faecium, Strain ERV102 | NWP80 | DEFB130B |
| Corynebacterium sp., Strain HFH0082 | NWP61 | PRND |
| Clostridiales bacterium, Strain 3_1_39B/D5 | NWP240 | TMEM119 |
| Klebsiella pneumoniae subsp. pneumoniae, Strain WGLW5 | NWP55 | PF4V1 |
| Escherichia coli, Strain MS 115-1 | NWP36 | DEFB130A |
| Lactobacillus jensenii, Strain 208-1 | NWP111 | BTC |
| Staphylococcus warneri A11 | warneriA11 | SLC8B1 |
| Bacillus sp., Strain DNF00233 | NWP90 | DEFB115 |
| Streptococcus sp., Strain AS20 | NWP116 | CTRB1 |
| Lactobacillus crispatus, Strain 125-2-CHN | NWP99 | CTRB2 |
| Staphylococcus warneri D9 | warneriD9 | RTBDN |
| Staphylococcus pasteuri C9 | pasteuriC9 | MICB |
| Proteus mirabilis, Strain WGLW6 | NWP59 | LCN2 |
| Streptococcus gallolyticus subsp. gallolyticus, Strain TX20005 | NWP27 | NKAI1_Epitope-2 |
| Veillonella sp., Oral Taxon 780, Strain F0422 | NWP268 | KCMA1_Epitope-1 |
| Fusobacterium nucleatum, Strain CTI-05 | NWP188 | IGFBP5 |
| Staphylococcus hominis C10 | hominisC10 | LOC644613 |
| Staphylococcus epidermidis C3 | EpiC3 | CLCC1 |
| Staphylococcus saprophyticus E8 | saprophyticusE8 | TFPI2 |
| Escherichia coli, Strain MS 115-1 | NWP36 | FGF17 |
| Fusobacterium sp., Strain CM21 | NWP166 | FAM19A3 |
| Prevotella buccae, Strain D17 | NWP256 | C6orf25 |
| Propionibacterium acnes, Strain HL046PA2 | NWP266 | FAM174A |
| Staphylococcus pasteuri C9 | pasteuriC9 | FAT2 |
| Streptococcus sobrinus, Strain W1703 | NWP85 | PAP |
| Staphylococcus warneri E6 | warneriE6 | ADRA2C |
| Streptococcus downei, Oral Taxon 594, Strain F0415 | NWP123 | BTC |
| Streptococcus mitis, Strain F0392 | NWP26 | SEMG1 |
| Staphylococcus warneri E4 | warneriE4 | SFRP4 |
| Escherichia coli, Strain MS 115-1 | NWP36 | LECT2 |
| Escherichia coli, Strain MS 196-1 | NWP42 | DEFB130A |
| Propionibacterium acnes, Strain HL002PA3 | NWP219 | CNGB3_Epitope-3 |
| Staphylococcus warneri B9 | warneriB9 | CP |
| Proteus mirabilis, Strain WGLW6 | NWP59 | ADCYAP1R1 |
| Arthrobacter albus, Strain DNF00011 | NWP86 | ALPP |
| Propionibacterium acnes, Strain HL043PA1 | NWP303 | FAM174A |
| Corynebacterium sp., Strain HFH0082 | NWP61 | MUC7 |
| Prevotella veroralis, Oral Taxon 572, Strain F0319 | NWP155 | PDIA6 |
| Corynebacterium amycolatum, Strain SK46 | NWP07 | C4A |
| Klebsiella pneumoniae subsp. pneumoniae, Strain WGLW1 | NWP56 | TMEM119 |
| Staphylococcus warneri C5 | warneriC5 | LOC644613 |
| Clostridium clostridioforme, Strain 2_1_49FAA | NWP228 | TNFSF10 |
| Hungatella hathewayi, Strain WAL-18680 | NWP231 | IL29 |
| Eubacterium sp., Strain 3_1_31 | NWP182 | MUCL3 |
| Peptoniphilus sp., Oral Taxon 836, Strain F0141 | NWP313 | ADM2 |
| Staphylococcus capitis E12 | capitisE12 | PDGFB |
| Klebsiella oxytoca, Strain MIT 10-5250 | NWP53 | CHGA |
| Proteus mirabilis, Strain WGLW6 | NWP59 | SCN4A_Epitope-3 |
| Lactobacillus gasseri, Strain JV-V03 | NWP106 | MUCL3 |
| Bacteroides stercoris, Strain CC31F | NWP270 | SCGB1C2 |
| Propionibacterium acnes, Strain HL027PA2 | NWP218 | CNGB3_Epitope-3 |
| Bacteroides stercoris, Strain CC31F | NWP270 | LCN1 |
| Propionibacterium acnes, Strain HL001PA1 | NWP203 | PROM2_Epitope-2 |
| Blautia sp., Strain AAB30 | AB30 | FAM19A3 |
| Streptococcus sp., Strain ACS2 | NWP115 | LINC00527 |
| Megasphaera micronuciformis, Strain DNF00954 | NWP307 | COL10A1 |
| Staphylococcus pasteuri F12 | pasteuriF12 | SERPINA11 |
| Streptococcus sp., Strain AS20 | NWP116 | PLA2G2E |
| Fusobacterium periodonticum, Strain 1_A_54/D10 | NWP142 | NGFR |
| Streptococcus anginosus, Strain F0211 | NWP125 | FGF17 |
| Proteus mirabilis, Strain WGLW6 | NWP59 | CRELD2 |
| Escherichia coli, Strain MS 85-1 | NWP30 | C2orf66 |
| Proteus mirabilis, Strain WGLW6 | NWP59 | RCN3 |
| Staphylococcus capitis A7 | capitisA7 | CNPY3 |
| Blautia sp., Strain AAB31 | AB31 | COL10A1 |
| Staphylococcus epidermidis G10 | EpiG10 | SMR3A |
| Bacteroides stercoris, Strain CC31F | NWP270 | CLEC4M |
| Staphylococcus pasteuri G3 | pasteuriG3 | FAT2 |
| Escherichia coli, Strain 83972 | NWP04 | PRR27 |
| Klebsiella oxytoca, Strain MIT 10-5249 | NWP52 | LCN2 |
| Staphylococcus aureus, Strain MN8 | NWP17 | SHISA7 |
| Staphylococcus warneri E6 | warneriE6 | PSG4 |
| Corynebacterium sp., Strain HFH0082 | NWP61 | LECT2 |
| Escherichia coli, Strain MS 119-7 | NWP31 | TMEM119 |
| Fusobacterium nucleatum subsp. animalis, Oral Taxon 420, Strain F0419 | NWP196 | EPHA4 |
| Enterococcus faecium, Strain ERV102 | NWP80 | NPPA |
| Staphylococcus epidermidis, Strain NIHLM031 | NWP73 | CCL2 |
| Staphylococcus capitis A7 | capitisA7 | FGFR1 |
| Peptostreptococcus sp., Strain CC14N | NWP290 | SMR3A |
| Ruminococcus gnavus, Strain NWP326 | RG109 | CCL8 |
| Erysipelotrichaceae sp., Strain 6_1_45 | NWP174 | CCL23 |
| Staphylococcus warneri D11 | warneriD12 | OLR1 |
| Bacteroides stercoris, Strain CC31F | NWP270 | CSF3 |
| Staphylococcus gallinarum E9 | gallinarumE9 | LOC644613 |
| Staphylococcus pasteuri F11 | pasteuriF11 | FAM174A |
| Deinococcus grandis, Strain SK125 | NWP89 | DEFB130B |
| Streptococcus sp., Strain OBRC6 | NWP124 | LOC644613 |
| Propionibacterium acnes, Strain SK137 | NWP309 | PGLYRP1 |
| Escherichia coli, Strain 83972 | NWP04 | TRPM5 |
| Streptococcus parasanguinis, Strain F0449 | NWP114 | UNQ6190/PRO20217 |
| Staphylococcus warneri, Strain SK66 | NWP11 | EPO |
| Bacillus sp., Strain DNF00233 | NWP90 | MUC7 |
| Fusobacterium nucleatum, Strain CTI-03 | NWP187 | CCL5 |
| Oscillibacter sp., Strain KLE-1728 | NWP269 | IL8RB |
| Staphylococcus warneri E6 | warneriE6 | CSMT1_Epitope-1 |
| Propionibacterium acnes, Strain HL045PA1 | NWP265 | CNGB3_Epitope-3 |
| Staphylococcus capitis A7 | capitisA7 | CSPG5 |
| Enterococcus faecium, Strain TX1330 | NWP19 | CSN3 |
| Propionibacterium acnes, Strain HL007PA1 | NWP225 | CNGB3_Epitope-3 |
| Fusobacterium sp., Strain CM21 | NWP166 | CTRB1 |
| Staphylococcus saprophyticus E8 | saprophyticusE8 | P2RX5 |
| Prevotella sp., Oral Taxon 472, Strain F0295 | NWP191 | FAM174A |
| Propionibacterium acnes, Strain SK137 | NWP309 | FAM174A |
| Escherichia coli, Strain 83972 | NWP04 | NPPA |
| Sporosarcina sp., Strain 2681 | NWP29 | FGF17 |
| Corynebacterium sp., Strain HFH0082 | NWP61 | CONA1_Epitope-1 |
| Prevotella bivia, Strain GED7760C | NWP233 | EPHA5 |
| Klebsiella sp., Strain MS 92-3 | NWP40 | SPOCK1 |
| Lachnospiraceae sp., Strain DNF00160 | NWP150 | LOC644613 |
| Streptococcus parasanguinis, Strain F0449 | NWP114 | CTRB2 |
| Streptococcus sp., Strain C150 | NWP121 | AGER |
| Staphylococcus capitis C4 | capitisC4 | COL10A1 |
| Streptococcus salivarius, Strain SK126 | NWP118 | PF4V1 |
| Klebsiella oxytoca, Strain MIT 10-5249 | NWP52 | A1BG |
| Bacteroides stercoris, Strain CC31F | NWP270 | IFNA17 |
| Streptococcus mitis, Strain F0392 | NWP26 | AGRP |
| Staphylococcus epidermidis, Strain NIHLM020 | NWP74 | FGF17 |
| Staphylococcus capitis C4 | capitisC4 | DEFB130B |
| Prevotella histicola, Strain F0411 | NWP144 | NKAI1_Epitope-2 |
| Staphylococcus warneri B9 | warneriB9 | FGFR2 |
| Micrococcus luteus, Strain SK58 | NWP08 | SEMG1 |
| Propionibacterium acnes, Strain HL050PA3 | NWP249 | TMEM119 |
| Bacillus sp., Strain DNF00233 | NWP90 | RCN1 |
| Bacillus sp., Strain DNF00233 | NWP90 | IGFBP5 |
| Staphylococcus aureus, Strain MN8 | NWP17 | C1QL3 |
| Escherichia coli, Strain MS 110-3 | NWP35 | LECT2 |
| Fusobacterium nucleatum, Strain CTI-02 | NWP186 | CCL18 |
| Blautia sp., Strain AAB30 | AB30 | CHRDL2 |
| Streptococcus mitis, Strain F0392 | NWP26 | DEFB130A |
| Staphylococcus lugdunensis F8 | lugdundensisF8 | BTC |
| Staphylococcus warneri E6 | warneriE6 | MTNR1B |
| Staphylococcus epidermidis, Strain NIH04008 | NWP77 | FAM19A3 |
| Proteus mirabilis, Strain WGLW4 | NWP58 | SYCN |
| Prevotella histicola, Strain F0411 | NWP144 | MUCL3 |
| Bacteroides sp., Strain 4_1_36 | NWP312 | COL10A1 |
| Bacteroides eggerthii, Strain 1_2_48FAA | NWP180 | RCN1 |
| Staphylococcus capitis B8 | capitisB8 | NPPC |
| Lactobacillus gasseri, Strain JV-V03 | NWP106 | SLPI |
| Staphylococcus warneri, Strain SK66 | NWP11 | SLC6A9 |
| Staphylococcus capitis A7 | capitisA7 | DKK4 |
| Bacteroides sp., Strain D2 | NWP192 | MUC7 |
| Sporosarcina sp., Strain 2681 | NWP29 | FAM19A2 |
| Staphylococcus capitis C4 | capitisC4 | NPPC |
| Staphylococcus hominis D2 | hominisD2 | SLC8B1 |
| Staphylococcus capitis A7 | capitisA7 | IL21 |
| Escherichia coli, Strain MS 85-1 | NWP30 | FGF17 |
| Staphylococcus warneri D11 | warneriD12 | EPHA6 |
| Lactobacillus gasseri, Strain EX33690VC16 | NWP113 | TMEM119 |
| Lactobacillus jensenii, Strain EX849587VC03 | NWP108 | LOC644613 |
| Fusobacterium sp., Strain CM22 | NWP167 | CCR9 |
| Lactobacillus jensenii, Strain 269-3 | NWP110 | COL26A1 |
| Staphylococcus pasteuri E5 | pasteuriE5 | MCFD2 |
| Bacteroides sp., Strain AAB8 | AB8 | LILRA3 |
| Staphylococcus warneri D4 | warneriD4 | DRAXIN |
| Megasphaera sp., Strain DNF00912 | NWP296 | PDIA3 |
| Staphylococcus warneri E6 | warneriE6 | CSF2RA |
| Staphylococcus pasteuri F11 | pasteuriF11 | MCFD2 |
| Staphylococcus pasteuri F11 | pasteuriF11 | FAT2 |
| Lactobacillus parafarraginis, Strain F0439 | NWP105 | ASIP |
| Propionibacterium acnes, Strain HL060PA1 | NWP239 | GPR37 |
| Lachnospiraceae sp., Strain ICM7 | NWP241 | IGFBP6 |
| Prevotella disiens, Strain DNF00882 | NWP177 | PTPRR |
| Lactobacillus crispatus, Strain JV-V01 | NWP107 | FP248 |
| Enterococcus faecalis, Strain S613 | NWP119 | AVP |
| Propionibacterium acnes, Strain HL038PA1 | NWP235 | SLC22A31 |
| Enterococcus faecium, Strain ERV99 | NWP82 | TMEM119 |
| Peptoniphilus sp., Oral Taxon 386, Strain F0131 | NWP255 | S28A1_Epitope-6 |
| Plesiomonas sp., Strain HPP0020 | NWP62 | FAM19A2 |
| Staphylococcus warneri E6 | warneriE6 | OLR1 |
| Lactobacillus crispatus, Strain GED7749C | NWP104 | INSL3 |
| Escherichia sp., Strain 3_2_53FAA | NWP02 | NPPA |
| Staphylococcus capitis A7 | capitisA7 | VSTM2B |
| Bacteroides sp., Strain D2 | NWP192 | DRAXIN |
| Micrococcus luteus, Strain SK58 | NWP08 | FGF19 |
| Bacteroides sp., Strain D2 | NWP192 | DEFB130B |
| Streptococcus sp., Strain ACS2 | NWP115 | ACP4 |
| Propionibacterium acnes, Strain HL013PA2 | NWP217 | PDCD1 |
| Staphylococcus epidermidis D11 | EpiD11 | XG |
| Citrobacter freundii, Strain 4_7_47CFAA | NWP88 | SPINK9 |
| Enterococcus faecium, Strain ERV165 | NWP81 | RTBDN |
| Peptoniphilus lacrimalis, Strain DNF00528 | NWP304 | PLA2G2E |
| Propionibacterium acnes, Strain HL038PA1 | NWP235 | CNGB3_Epitope-3 |
| Bacteroides sp., Strain D2 | NWP192 | COL10A1 |
| Clostridium orbiscindens, Strain CC43_001K | NWP259 | PDIA2 |
| Staphylococcus aureus, Strain CM05 | NWP83 | IGF1 |
| Prevotella sp., Oral Taxon 472, Strain F0295 | NWP191 | PSORS1C2 |
| Propionibacterium acnes, Strain HL013PA2 | NWP217 | CNGB3_Epitope-3 |
| Fusobacterium nucleatum, Strain CTI-06 | NWP189 | AGER |
| Fusobacterium nucleatum subsp. animalis, Oral Taxon 420, Strain F0419 | NWP196 | IL29 |
| Bacteroides sp., Strain D2 | NWP192 | KLK10 |
| Streptococcus salivarius, Strain SK126 | NWP118 | DEFB130B |
| Staphylococcus haemolytics, Strain DNF00585 | NWP87 | TMEM119 |
| Lachnospiraceae bacterium, Strain AAB25 | AB25 | MUCL3 |
| Staphylococcus pasteuri F12 | pasteuriF12 | FAT2 |
| Fusobacterium sp., Oral Taxon 370, Strain F0437 | NWP143 | SIRPA |
| Fusobacterium periodonticum, Strain 2_1_31 | NWP160 | LAIR1 |
| Eggerthella sp., Strain MVA1 | NWP185 | MUC7 |
| Staphylococcus aureus, Strain MN8 | NWP17 | SLC8B1 |
| Staphylococcus warneri D11 | warneriD12 | SLC8B1 |
| Streptococcus anginosus, Strain F0211 | NWP125 | CCL17 |
| Prevotella bivia, Strain GED7760C | NWP233 | CCL17 |
| Staphylococcus capitis A7 | capitisA7 | DCN |
| Streptococcus sp., Strain ACS2 | NWP115 | IFNA5 |
| Peptoniphilus sp., Oral Taxon 836, Strain F0141 | NWP313 | SPINK9 |
| Fusobacterium periodonticum, Strain 2_1_31 | NWP160 | FKBP2 |
| Staphylococcus warneri D4 | warneriD4 | RTBDN |
| Staphylococcus epidermidis, Strain NIHLM018 | NWP72 | TMEM119 |
| Escherichia coli, Strain MS 110-3 | NWP35 | CHRDL2 |
| Staphylococcus hominis, Strain C80 | NWP25 | FGF17 |
| Staphylococcus warneri, Strain SK66 | NWP11 | CXCR4 |
| Streptococcus parasanguinis, Strain F0449 | NWP114 | C1QL2 |
| Propionibacterium acnes, Strain HL078PA1 | NWP305 | PGLYRP1 |
| Proteus mirabilis, Strain WGLW6 | NWP59 | CXCR5 |
| Corynebacterium amycolatum, Strain SK46 | NWP07 | DRAXIN |
| Corynebacterium sp., Strain HFH0082 | NWP61 | SEMG2 |
| Streptococcus sp., Strain OBRC6 | NWP124 | SHISA6 |
| Streptococcus anginosus, Strain F0211 | NWP125 | UNQ6190/PRO20217 |
| Staphylococcus aureus, Strain MN8 | NWP17 | KCNK4_Epitope-1 |
| Lactobacillus jensenii, Strain 208-1 | NWP111 | CCL17 |
| Fusobacterium sp., Strain CM22 | NWP153 | SLC29A4 |
| Escherichia coli, Strain MS 16-3 | NWP37 | NPPA |
| Staphylococcus hominis B3 | hominisB3 | TNFRSF21 |
| Staphylococcus warneri C5 | warneriC5 | MUCL3 |
| Fusobacterium nucleatum, Strain CTI-02 | NWP186 | NPPC |
| Streptococcus sp., Strain SR4 | NWP130 | CTRB1 |
| Staphylococcus warneri B9 | warneriB9 | TENM1 |
| Streptococcus sp., Strain C150 | NWP121 | FGFRL1 |
| Staphylococcus pasteuri E5 | pasteuriE5 | CSPG5 |
| Citrobacter sp., Strain 30_2 | NWP01 | CCL5 |
| Staphylococcus capitis B12 | capitisB12 | CSPG5 |
| Corynebacterium amycolatum, Strain SK46 | NWP07 | KLK10 |
| Hungatella hathewayi, Strain WAL-18680 | NWP231 | HTRA3 |
| Enterococcus faecium, Strain TX0133a04 | NWP44 | PDGFB |
| Propionibacterium acnes, Strain HL053PA2 | NWP236 | CNGB3_Epitope-3 |
| Streptococcus salivarius, Strain SK126 | NWP118 | ASIP |
| Propionibacterium acnes, Strain HL053PA1 | NWP260 | SLC22A31 |
| Sporosarcina sp., Strain 2681 | NWP29 | C2orf66 |
| Streptococcus parasanguinis, Strain F0449 | NWP114 | PEBP4 |
| Escherichia coli, Strain MS 16-3 | NWP37 | PLA2G2E |
| Staphylococcus capitis E7 | capitisE7 | SMR3A |
| Klebsiella pneumoniae subsp. pneumoniae, Strain WGLW3 | NWP54 | TMEM119 |
| Streptococcus parasanguinis, Strain F0449 | NWP114 | OTOL1 |
| Allobaculum sp., Strain NWP324 | Ery128 | SLC22A4 |
| Fusobacterium periodonticum, Strain 2_1_31 | NWP160 | APOO |
| Escherichia coli, Strain MS 110-3 | NWP35 | COL10A1 |
| Clostridium cadaveris, Strain CC44_001G | NWP247 | PF4V1 |
| Staphylococcus pasteuri G7 | pasteuriG7 | FAT2 |
| Staphylococcus warneri B9 | warneriB9 | ADCY5 |
| Propionibacterium acnes, Strain HL036PA1 | NWP226 | SLC22A31 |
| Streptococcus sp., Strain OBRC6 | NWP124 | FAM19A3 |
| Propionibacterium acnes, Strain HL036PA2 | NWP227 | CNGB3_Epitope-3 |
| Klebsiella pneumoniae subsp. pneumoniae, Strain WGLW1 | NWP56 | PLA2G2E |
| Collinsella sp., Strain 4_8_47FAA | NWP198 | CCL18 |
| Propionibacterium acnes, Strain HL025PA1 | NWP204 | CNGB3_Epitope-3 |
| Catabacter hongkongensis, Strain AB8_9 | NWP299 | RBP4 |
| Prevotella disiens, Strain DNF00882 | NWP177 | GP6 |
| Staphylococcus saprophyticus E8 | saprophyticusE8 | TNFRSF13B |
| Corynebacterium sp., Strain CMW7794 | NWP98 | LOC644613 |
| Coprococcus sp., Strain AAb34 | AB34 | DEFB130B |
| Staphylococcus warneri C2 | warneriC2 | RTBDN |
| Staphylococcus warneri, Strain SK66 | NWP11 | PRRT1 |
| Streptococcus gallolyticus subsp. gallolyticus, Strain TX20005 | NWP27 | INSL3 |
| Bacteroides sp., Strain 2_1_16 | NWP273 | AVP |
| Staphylococcus pasteuri E1 | pasteuriE1 | GYPA |
| Staphylococcus xylosus F2 | xylosusF2 | MUCL3 |
| Staphylococcus cohnii G11 | cohniiG11 | XG |
| Staphylococcus epidermidis, Strain NIH04008 | NWP77 | CCL2 |
| Proteus mirabilis, Strain WGLW6 | NWP59 | SLC39A14 |
| Lactobacillus gasseri, Strain EX33690VC16 | NWP113 | TAS1R3 |
| Staphylococcus capitis B8 | capitisB8 | LECT2 |
| Staphylococcus capitis E7 | capitisE7 | NPPC |
| Shigella sp., Strain D9 | NWP06 | FAM19A3 |
| Pseudomonas sp., Strain HPB0071 | NWP65 | CCL18 |
| Staphylococcus capitis B1 | capitisB1 | MUCL3 |
| Proteus mirabilis, Strain WGLW4 | NWP58 | UNQ6494/PRO21346 |
| Staphylococcus saprophyticus D10 | saprophyticusD10 | LOC644613 |
| Clostridium sp., Strain MSTE9 | NWP253 | CCL18 |
| Streptococcus sp., Strain SR1 | NWP122 | TMEM119 |
| Eubacterium sp., Strain 3_1_31 | NWP182 | CSAG1 |
| Propionibacterium acnes, Strain HL005PA2 | NWP215 | PDCD1 |
| Rhodococcus erythropolis, Strain SK121 | NWP95 | PIANP |
| Staphylococcus epidermidis, Strain NIH04008 | NWP77 | PIANP |
| Micrococcus luteus, Strain SK58 | NWP08 | S39A6_Epitope-1 |
| Streptococcus sp., Strain AS20 | NWP116 | COL10A1 |
| Veillonella sp., Oral Taxon 780, Strain F0422 | NWP268 | PDIA2 |
| Bacteroides sp., Strain 2_1_16 | NWP273 | ENDOU |
| Streptococcus sp., Strain ACS2 | NWP115 | CTL4_Epitope-3 |
| Fusobacterium sp., Strain CM22 | NWP167 | CLU |
| Escherichia coli, Strain MS 145-7 | NWP33 | CLEC4G |
| Propionibacterium acnes, Strain HL027PA1 | NWP211 | FAM174A |
| Staphylococcus pasteuri F11 | pasteuriF11 | SERPINA11 |
| Clostridium difficile, Strain 70-100-2010 | NWP279 | PF4V1 |
| Escherichia coli, Strain MS 69-1 | NWP39 | PRR27 |
| Sporosarcina sp., Strain 2681 | NWP29 | LECT2 |
| Streptococcus anginosus, Strain F0211 | NWP125 | TMEM149 |
| Weissella cibaria, Strain F16_1 | NWP101 | BTC |
| Staphylococcus warneri B9 | warneriB9 | PLA2G3 |
| Staphylococcus warneri, Strain SK66 | NWP11 | LOC644613 |
| Staphylococcus capitis A7 | capitisA7 | OR1L6_Epitope-1 |
| Lactobacillus crispatus, Strain GED7749C | NWP104 | TFF3 |
| Streptococcus mitis, Strain F0392 | NWP26 | LECT2 |
| Staphylococcus epidermidis, Strain NIH04008 | NWP77 | FLT3LG |
| Staphylococcus aureus, Strain TCH70 | NWP12 | SCGB1A1 |
| Staphylococcus capitis A7 | capitisA7 | KCNMB2 |
| Enterococcus faecium, Strain TX0133a04 | NWP44 | UNQ6190/PRO20217 |
| Escherichia coli, Strain MS 196-1 | NWP42 | SMR3A |
| Streptococcus parasanguinis, Strain F0449 | NWP114 | CONA1_Epitope-1 |
| Rothia mucilaginosa, Strain CC87LB | NWP84 | TMEM119 |
| Streptococcus gallolyticus subsp. gallolyticus, Strain TX20005 | NWP27 | SLC22A31 |
| Proteus mirabilis, Strain WGLW6 | NWP59 | CHRNA9 |
| Staphylococcus capitis A7 | capitisA7 | MTNR1B |
| Bacteroides stercoris, Strain CC31F | NWP270 | SCGB1A1 |
| Staphylococcus capitis A7 | capitisA7 | TMC3 |
| Klebsiella pneumoniae subsp. pneumoniae, Strain WGLW1 | NWP56 | NPPA |
| Lachnospiraceae bacterium, Strain AAB23 | AB23 | COLEC12 |
| Staphylococcus hominis C10 | hominisC10 | MUCL3 |
| Megasphaera sp., Strain DNF00912 | NWP296 | PRR27 |
| Staphylococcus capitis B8 | capitisB8 | NPPA |
| Fusobacterium nucleatum subsp. animalis, Oral Taxon 420, Strain F0419 | NWP196 | FGF1 |
| Escherichia coli, Strain MS 110-3 | NWP35 | PRND |
| Escherichia sp., Strain 3_2_53FAA | NWP02 | AGRP |
| Eggerthella sp., Strain MVA1 | NWP185 | CSAG1 |
| Enterococcus faecium, Strain ERV165 | NWP81 | C4A |
| Staphylococcus saprophyticus D1 | saprophyticusD1 | ENDOU |
| Staphylococcus pasteuri G3 | pasteuriG3 | TNMD |
| Fusobacterium nucleatum, Strain MJR7757B | NWP257 | EPHA5 |
| Staphylococcus epidermidis H11 | EpiH11 | XG |
| Lactobacillus jensenii, Strain 269-3 | NWP110 | KIR3DS1 |
| Staphylococcus warneri B9 | warneriB9 | DQA2_Epitope-1 |
| Klebsiella oxytoca, Strain MIT 10-5246 | NWP50 | CHGA |
| Lactobacillus gasseri, Strain 224-1 | NWP112 | SHISA2 |
| Enterococcus faecium, Strain TX1330 | NWP19 | PF4V1 |
| Staphylococcus pasteuri E5 | pasteuriE5 | RTN4R |
| Streptococcus intermedius, Strain F0413 | NWP128 | TSLP |
| Staphylococcus haemolyticus B6 | haemolyticusB6 | PF4V1 |
| Enterococcus faecalis, Strain TX2137 | NWP117 | AVP |
| Staphylococcus capitis B8 | capitisB8 | DEFB130B |
| Corynebacterium sp., Strain HFH0082 | NWP61 | FAM19A2 |
| Staphylococcus capitis C4 | capitisC4 | PIANP |
| Fusobacterium nucleatum, Strain CTI-05 | NWP188 | LOC644613 |
| Streptococcus mitis, Strain F0392 | NWP26 | MUC7 |
| Escherichia coli, Strain MS 145-7 | NWP33 | GPR142 |
| Lactobacillus jensenii, Strain 269-3 | NWP110 | SLC8B1 |
| Corynebacterium amycolatum, Strain SK46 | NWP07 | SEMG2 |
| Prevotella bivia, Strain DNF00650 | NWP176 | AGER |
| Staphylococcus warneri, Strain SK66 | NWP11 | IGFBP4 |
| Streptococcus salivarius, Strain SK126 | NWP118 | RTBDN |
| Staphylococcus warneri D11 | warneriD12 | OR1L1_Epitope-1 |
| Lactobacillus jensenii, Strain 208-1 | NWP111 | PEBP4 |
| Fusobacterium sp., Strain CM22 | NWP167 | RCN3 |
| Propionibacterium acnes, Strain HL005PA3 | NWP216 | CNGB3_Epitope-3 |
| Staphylococcus epidermidis, Strain NIHLM070 | NWP69 | TMEM119 |
| Propionibacterium acnes, Strain HL060PA1 | NWP239 | TMEM119 |
| Staphylococcus saprophyticus D6 | saprophyticusD6 | PDGFB |
| Micrococcus luteus, Strain SK58 | NWP08 | CONA1_Epitope-1 |
| Staphylococcus pasteuri E1 | pasteuriE1 | HCG22 |
| Staphylococcus aureus, Strain CM05 | NWP83 | SO1B7_Epitope-5 |
| Fusobacterium nucleatum subsp. animalis, Oral Taxon 420, Strain F0419 | NWP196 | SLPI |
| Sporosarcina sp., Strain 2681 | NWP29 | SEMG2 |
| Staphylococcus capitis A7 | capitisA7 | EPHA6 |
| Streptococcus sp., Strain ACS2 | NWP115 | GPR151 |
| Corynebacterium sp., Strain CMW7794 | NWP98 | FAM19A3 |
| Escherichia coli, Strain MS 85-1 | NWP30 | IGFBP5 |
| Blautia sp., Strain AAB30 | AB30 | FAM19A2 |
| Prevotella sp., Oral Taxon 317, Strain F0108 | NWP173 | IBSP |
| Clostridium perfringens, Strain WAL-14572 | NWP311 | KISS1 |
| Clostridium aldenense, Strain WAL-18727 | NWP314 | FAM19A3 |
| Staphylococcus warneri, Strain SK66 | NWP11 | ADRA2C |
| Enterococcus faecium, Strain TX0133a04 | NWP44 | LOC644613 |
| Klebsiella oxytoca, Strain MIT 10-5242 | NWP46 | DEFA6 |
| Lactobacillus crispatus, Strain 125-2-CHN | NWP99 | BTC |
| Enterococcus faecium, Strain TX1330 | NWP19 | SEMG2 |
| Lactobacillus jensenii, Strain EX849587VC06 | NWP109 | SMR3A |
| Streptococcus sp., Strain AS20 | NWP116 | NPPC |
| Sporosarcina sp., Strain 2681 | NWP29 | TRPM5 |
| Corynebacterium amycolatum, Strain SK46 | NWP07 | FGF17 |
| Escherichia coli, Strain 83972 | NWP04 | C4A |
| Streptococcus gallolyticus subsp. gallolyticus, Strain TX20005 | NWP27 | PDCD1 |
| Bacteroides eggerthii, Strain 1_2_48FAA | NWP180 | CALU |
| Bacteroides dorei, Strain 5_1_36/D4 | NWP244 | RBP4 |
| Prevotella sp., Oral Taxon 472, Strain F0295 | NWP191 | SLC10A4 |
| Staphylococcus capitis E7 | capitisE7 | NPPA |
| Staphylococcus warneri E6 | warneriE6 | SPRN |
| Prevotella bivia, Strain DNF00188 | NWP193 | C2orf40 |
| Staphylococcus warneri B9 | warneriB9 | CXCR4 |
| Escherichia coli, Strain MS 145-7 | NWP33 | PIANP |
| Clostridiales bacterium, Strain 4_1_37FAA | NWP285 | TXNDC12 |
| Lactobacillus crispatus, Strain MV-3A-US | NWP103 | P4HB |
| Enterococcus faecium, Strain ERV102 | NWP80 | RTBDN |
| Bacteroides fragilis, Strain CL07T12C05 | NWP282 | TSPAN8 |
| Bacteroides sp., Strain D2 | NWP192 | FAM19A2 |
| Fusobacterium nucleatum subsp. animalis, Oral Taxon 420, Strain F0419 | NWP196 | GNLY |
| Prevotella sp., Oral Taxon 472, Strain F0295 | NWP191 | SMR3B |
| Staphylococcus epidermidis, Strain NIHLM008 | NWP70 | TMEM119 |
| Citrobacter sp., Strain 30_2 | NWP01 | AGER |
| Staphylococcus capitis B8 | capitisB8 | PIANP |
| Ruminococcus gnavus, Strain AAB28 | AB28 | CSAG1 |
| Shigella sp., Strain D9 | NWP06 | YB043 |
| Plesiomonas sp., Strain HPP0020 | NWP62 | C4A |
| Staphylococcus capitis E7 | capitisE7 | PIANP |
| Propionibacterium propionicum, Oral Taxon 739, Strain F0230 | NWP301 | CD151 |
| Eubacterium infirmum, Strain F0142 | NWP223 | APOO |
| Staphylococcus warneri F1 | warneriF1 | F3 |
| Streptococcus cristatus, Strain F0329 | NWP126 | PF4V1 |
| Bacteroides sp., Strain AAB8 | AB8 | HFE |
| Parabacteroides sp., Strain D13 | NWP172 | AMY2A |
| Staphylococcus epidermidis C3 | EpiC3 | SMR3A |
| Sporosarcina sp., Strain 2681 | NWP29 | ADAMTS16 |
| Shigella sp., Strain D9 | NWP06 | PLA2G2E |
| Clostridiales bacterium, Strain 4_1_37FAA | NWP285 | MFSD2A |
| Propionibacterium acnes, Strain HL025PA2 | NWP210 | CNGB3_Epitope-3 |
| Escherichia coli, Strain MS 145-7 | NWP33 | AVP |
| Proteus mirabilis, Strain WGLW6 | NWP59 | NBL1 |
| Staphylococcus pasteuri C9 | pasteuriC9 | TNFSF6 |
| Sporosarcina sp., Strain 2681 | NWP29 | CSN3 |
| Staphylococcus capitis E11 | capitisE11 | CDSN |
| Streptococcus sp., Strain BS29a | NWP129 | CRTAC1 |
| Propionibacterium acnes, Strain HL007PA1 | NWP225 | SLC22A31 |
| Staphylococcus epidermidis, Strain NIHLM040 | NWP76 | PMCH |
| Corynebacterium sp., Strain CMW7794 | NWP98 | LECT2 |
| Staphylococcus warneri E4 | warneriE4 | CRELD1 |
| Propionibacterium acnes, Strain HL025PA1 | NWP204 | SPINT3 |
| Klebsiella pneumoniae subsp. pneumoniae, Strain WGLW1 | NWP56 | CHGA |
| Staphylococcus saprophyticus E8 | saprophyticusE8 | FCGR1A |
| Klebsiella sp., Strain MS 92-3 | NWP40 | FAM174A |
| Streptococcus sp., Strain OBRC6 | NWP124 | DEFB130B |
| Fusobacterium periodonticum, Strain 1_A_54/D10 | NWP142 | TNFRSF10B |
| Staphylococcus cohnii G11 | cohniiG11 | CD320 |
| Prevotella bivia, Strain DNF00650 | NWP176 | RCN2 |
| Streptococcus salivarius, Strain SK126 | NWP118 | PIANP |
| Staphylococcus intermedius F7 | intermediusF7 | KCMA1_Epitope-1 |
| Lactobacillus crispatus, Strain JV-V01 | NWP107 | TMEM119 |
| Enterococcus faecalis, Strain S613 | NWP119 | SYCN |
| Staphylococcus hominis B3 | hominisB3 | SOSTDC1 |
| Enterococcus faecium, Strain E417 | NWP79 | AGER |
| Corynebacterium amycolatum, Strain SK46 | NWP07 | ADAMTS16 |
| Arthrobacter albus, Strain DNF00011 | NWP86 | CTRB2 |
| Propionibacterium acnes, Strain HL037PA2 | NWP205 | PF4V1 |
| Propionibacterium acnes, Strain HL056PA1 | NWP267 | PDCD1 |
| Fusobacterium sp., Strain CM22 | NWP167 | NCKX2_Epitope-1 |
| Peptostreptococcus sp., Strain CC14N | NWP290 | FAM19A3 |
| Klebsiella oxytoca, Strain MIT 10-5246 | NWP50 | DEFA6 |
| Prevotella bivia, Strain DNF00188 | NWP193 | RCN2 |
| Prevotella bivia, Strain DNF00650 | NWP176 | RTBDN |
| Prevotella timonensis, Strain CRIS 5C-B1 | NWP156 | KCMA1_Epitope-1 |
| Escherichia coli, Strain MS 196-1 | NWP42 | PRR27 |
| Staphylococcus epidermidis F6 | EpiF6 | MUCL3 |
| Escherichia coli, Strain 83972 | NWP04 | ADAMTS16 |
| Propionibacterium acnes, Strain HL027PA1 | NWP211 | TMEM119 |
| Escherichia coli, Strain MS 110-3 | NWP35 | CONA1_Epitope-1 |
| Streptococcus mitis, Strain F0392 | NWP26 | FGF19 |
| Blautia sp., Strain KLE 1732 | NWP292 | CCL18 |
| Lactobacillus jensenii, Strain EX849587VC06 | NWP109 | PIANP |
| Collinsella aerofaciens, Strain AAB3 | AB3 | PIANP |
| Clostridium orbiscindens, Strain CC43_001K | NWP259 | TXNDC12 |
| Erysipelotrichaceae sp., Strain 6_1_45 | NWP181 | NPPA |
| Streptococcus sp., Strain C150 | NWP121 | CSAG1 |
| Fusobacterium nucleatum, Strain CTI-07 | NWP190 | S39A6_Epitope-1 |
| Staphylococcus warneri C2 | warneriC2 | MUCL3 |
| Deinococcus grandis, Strain SK125 | NWP89 | FAM19A3 |
| Staphylococcus pasteuri G3 | pasteuriG3 | SERPINA11 |
| Shigella sp., Strain D9 | NWP06 | KIRREL |
| Streptococcus salivarius, Strain SK126 | NWP118 | CTRB2 |
| Staphylococcus warneri B9 | warneriB9 | OPN3 |
| Corynebacterium sp., Strain HFH0082 | NWP61 | ADAMTS16 |
| Staphylococcus pasteuri F12 | pasteuriF12 | TNMD |
| Clostridium cadaveris, Strain CC44_001G | NWP247 | NKAI1_Epitope-2 |
| Staphylococcus capitis C12 | capitisC12 | LOC644613 |
| Streptococcus sobrinus, Strain W1703 | NWP85 | CCL17 |
| Proteus mirabilis, Strain WGLW4 | NWP58 | DRAXIN |
| Corynebacterium sp., Strain CMW7794 | NWP98 | DEFB130B |
| Staphylococcus caprae, Strain C87 | NWP24 | LGALS7 |
| Dermabacter sp., Strain HFH0086 | NWP92 | C3AR_Epitope-3 |
| Enterococcus faecium, Strain E417 | NWP79 | TMEM119 |
| Staphylococcus epidermidis F6 | EpiF6 | LOC644613 |
| Fusobacterium nucleatum, Strain CTI-07 | NWP190 | DKK1 |
| Erysipelotrichaceae sp., Strain 6_1_45 | NWP174 | AGRP |
| Proteus mirabilis, Strain WGLW6 | NWP59 | SCN9A_Epitope-3 |
| Klebsiella oxytoca, Strain MIT 10-5246 | NWP50 | SLC22A4 |
| Corynebacterium sp., Strain HFH0082 | NWP61 | FGF17 |
| Lactobacillus jensenii, Strain EX849587VC06 | NWP109 | TNFSF9 |
| Lactobacillus jensenii, Strain 208-1 | NWP111 | CSAG1 |
| Escherichia coli, Strain MS 110-3 | NWP35 | LMBRD1 |
| Streptococcus sp., Strain AS20 | NWP116 | ASIP |
| Clostridium bolteae, Strain CC43_001B | NWP245 | APOO |
| Lactobacillus jensenii, Strain EX849587VC03 | NWP108 | DEFB130A |
| Citrobacter sp., Strain 30_2 | NWP01 | C1QTNF2 |
| Eggerthella sp., Strain MVA1 | NWP185 | SMR3A |
| Staphylococcus warneri E4 | warneriE4 | EPO |
| Enterococcus faecium, Strain TX0133a04 | NWP44 | CTRB2 |
| Propionibacterium acnes, Strain HL053PA2 | NWP236 | SPINT3 |
| Klebsiella oxytoca, Strain MIT 10-5246 | NWP50 | SLC22A5 |
| Blautia sp., Strain KLE 1732 | NWP292 | PRR27 |
| Prevotella denticola, Strain DNF00960 | NWP179 | CTRB1 |
| Fusobacterium nucleatum, Strain CTI-05 | NWP188 | ADAMTS16 |
| Staphylococcus warneri D11 | warneriD12 | MEPE |
| Rhodococcus erythropolis, Strain SK121 | NWP95 | COL10A1 |
| Enterococcus faecium, Strain ERV165 | NWP81 | ADAMTS16 |
| Veillonella sp., Oral Taxon 780, Strain F0422 | NWP268 | TNFRSF10B |
| Proteus mirabilis, Strain WGLW4 | NWP58 | TMEM119 |
| Anaerostipes sp., Strain 3_2_56FAA | NWP277 | PDIA6 |
| Blautia sp., Strain AAB30 | AB30 | PRR27 |
| Corynebacterium amycolatum, Strain SK46 | NWP07 | TRPM5 |
| Staphylococcus pasteuri F12 | pasteuriF12 | MCFD2 |
| Escherichia coli, Strain MS 145-7 | NWP33 | MMP9 |
| Staphylococcus haemolyticus G2 | haemolyticusG2 | CD320 |
| Staphylococcus epidermidis, Strain NIHLM067 | NWP68 | TMEM119 |
| Staphylococcus warneri E6 | warneriE6 | MEPE |
| Enterococcus faecalis, Strain TX2137 | NWP117 | PF4V1 |
| Fusobacterium sp., Strain CM22 | NWP167 | GRPR |
| Streptococcus sp., Strain SR1 | NWP122 | PEBP4 |
| Prevotella disiens, Strain DNF00882 | NWP177 | IL22 |
| Fusobacterium sp., Strain CM21 | NWP166 | CSAG1 |
| Enterococcus faecalis, Strain R712 | NWP120 | AVP |
| Fusobacterium sp., Strain CM22 | NWP167 | CEACAM4 |
| Staphylococcus pasteuri G3 | pasteuriG3 | MCFD2 |
| Fusobacterium sp., Oral Taxon 370, Strain F0437 | NWP143 | AJAP1 |
| Staphylococcus epidermidis, Strain NIHLM015 | NWP71 | SCGB2B2 |
| Lactobacillus jensenii, Strain 208-1 | NWP111 | MANF |
| Escherichia coli, Strain MS 145-7 | NWP33 | NPFF2_Epitope-1 |
| Enterococcus faecium, Strain ERV165 | NWP81 | DRAXIN |
| Bacteroidales bacterium, Strain KA00344 | NWP302 | IFNL2 |
| Staphylococcus warneri E6 | warneriE6 | FGF1 |
| Escherichia coli, Strain MS 200-1 | NWP41 | TMEM119 |
| Klebsiella oxytoca, Strain MIT 10-5245 | NWP49 | PDZD11 |
| Klebsiella oxytoca, Strain MIT 10-5245 | NWP49 | TRPM1_Epitope-2 |
| Prevotella bivia, Strain DNF00650 | NWP176 | RCN3 |
| Klebsiella oxytoca, Strain MIT 10-5249 | NWP52 | INSL4 |
| Proteus mirabilis, Strain WGLW6 | NWP59 | CLU |
| Escherichia coli, Strain 83972 | NWP04 | IGFBP5 |
| Staphylococcus pasteuri F12 | pasteuriF12 | TNFRSF21 |
| Lactobacillus gasseri, Strain JV-V03 | NWP106 | FAM19A2 |
| Staphylococcus capitis A7 | capitisA7 | PTH1R |
| Escherichia coli, Strain 83972 | NWP04 | AHSG |
| Catabacter hongkongensis, Strain AB8_9 | NWP299 | SMR3A |
| Staphylococcus epidermidis, Strain NIHLM015 | NWP71 | CCL2 |
| Propionibacterium acnes, Strain HL045PA1 | NWP265 | PDCD1 |
| Lactobacillus crispatus, Strain JV-V01 | NWP107 | MFSD8_Epitope-5 |
| Staphylococcus warneri B9 | warneriB9 | IGFBP4 |
| Staphylococcus aureus, Strain TCH70 | NWP12 | IGF1 |
| Propionibacterium acnes, Strain HL037PA1 | NWP208 | CNGB3_Epitope-3 |
| Proteus mirabilis, Strain WGLW6 | NWP59 | EPHA5 |
| Lactobacillus jensenii, Strain EX849587VC06 | NWP109 | TFF1 |
| Shuttleworthia sp., Strain MSX8B | NWP149 | OSTN |
| Bacteroides fragilis, Strain CL07T12C05 | NWP282 | SHISA2 |
| Staphylococcus aureus, Strain CM05 | NWP83 | SCGB1A1 |
| Plesiomonas sp., Strain HPP0020 | NWP62 | TRPM5 |
| Citrobacter sp., Strain 30_2 | NWP01 | TMEM119 |
| Fusobacterium sp., Oral Taxon 370, Strain F0437 | NWP143 | AGER |
| Staphylococcus capitis E7 | capitisE7 | DEFB130B |
| Enterococcus faecium, Strain TX1330 | NWP19 | PRND |
| Propionibacterium acnes, Strain HL043PA2 | NWP264 | CNGB3_Epitope-3 |
| Parabacteroides merdae, Strain CL09T00C40 | NWP184 | CSAG1 |
| Propionibacterium acnes, Strain HL030PA2 | NWP207 | SPINT3 |
| Staphylococcus pasteuri F11 | pasteuriF11 | TNFRSF21 |
| Streptococcus sp., Strain BS21 | NWP131 | MUCL3 |
| Lactobacillus parafarraginis, Strain F0439 | NWP105 | ENDOU |
| Staphylococcus aureus, Strain CM05 | NWP83 | MSMB |
| Escherichia coli, Strain MS 115-1 | NWP36 | IGFBP5 |
| Actinomyces neuii, Strain MJR8396A | NWP258 | NRG2 |
| Actinomyces neuii, Strain MJR8396A | NWP258 | EPHA5 |
| Klebsiella oxytoca, Strain MIT 10-5249 | NWP52 | TMEM119 |
| Proteus mirabilis, Strain WGLW4 | NWP58 | GFRA1 |
| Propionibacterium acnes, Strain HL013PA2 | NWP217 | SPINK14 |
| Lactobacillus parafarraginis, Strain F0439 | NWP105 | SHISA6 |
| Proteus mirabilis, Strain WGLW6 | NWP59 | ITPR2_Epitope-3 |
| Staphylococcus warneri B9 | warneriB9 | TUSC5 |
| Streptococcus mitis, Strain F0392 | NWP26 | CONA1_Epitope-1 |
| Lactobacillus gasseri, Strain SV-16A-US | NWP102 | CCL5 |
| Staphylococcus warneri D4 | warneriD4 | GRIA2_Epitope-2 |
| Propionibacterium acnes, Strain HL001PA1 | NWP203 | NOG |
| Klebsiella oxytoca, Strain MIT 10-5248 | NWP51 | ACP4 |
| Propionibacterium acnes, Strain HL002PA2 | NWP214 | CNGB3_Epitope-3 |
| Fusobacterium periodonticum, Strain 1_A_54/D10 | NWP142 | BTN3A3 |
| Bacteroides stercoris, Strain CC31F | NWP270 | OTOR |
| Citrobacter freundii, Strain 4_7_47CFAA | NWP88 | GNLY |
| Streptococcus sp., Strain ACC21 | NWP134 | PIANP |
| Propionibacterium sp., Strain 5_U_42AFAA | NWP300 | TACSTD2 |
| Escherichia coli, Strain MS 196-1 | NWP42 | TRPM5 |
| Coprococcus sp., Strain AAb34 | AB34 | PIANP |
| Streptococcus gallolyticus subsp. gallolyticus, Strain TX20005 | NWP27 | GSG1L |
| Propionibacterium acnes, Strain HL046PA2 | NWP266 | PDCD1 |
| Staphylococcus warneri B9 | warneriB9 | HRC |
| Bacteroides stercoris, Strain CC31F | NWP270 | TXN |
| Propionibacterium acnes, Strain HL060PA1 | NWP239 | CNGB3_Epitope-3 |
| Propionibacterium acnes, Strain HL005PA2 | NWP215 | CNGB3_Epitope-3 |
| Staphylococcus caprae, Strain C87 | NWP24 | FGF17 |
| Escherichia coli, Strain MS 145-7 | NWP33 | COL10A1 |
| Klebsiella oxytoca, Strain MIT 10-5249 | NWP52 | ACVRL1 |
| Escherichia coli, Strain MS 69-1 | NWP39 | AGRP |
| Staphylococcus warneri B9 | warneriB9 | CGREF1 |
| Streptococcus sp., Strain ACS2 | NWP115 | AXL |
| Prevotella disiens, Strain DNF00882 | NWP177 | LUM |
| Klebsiella oxytoca, Strain MIT 10-5242 | NWP46 | LGALS7 |
| Citrobacter sp., Strain 30_2 | NWP01 | PTHLH |
| Collinsella sp., Strain 4_8_47FAA | NWP198 | IGFBP5 |
| Staphylococcus epidermidis, Strain NIHLM031 | NWP73 | UNQ6190/PRO20217 |
| Klebsiella pneumoniae subsp. pneumoniae, Strain WGLW3 | NWP54 | FAM19A3 |
| Proteus mirabilis, Strain WGLW4 | NWP58 | NPPA |
| Streptococcus anginosus, Strain F0211 | NWP125 | AXL |
| Escherichia coli, Strain MS 110-3 | NWP35 | C4A |
| Enterococcus faecium, Strain TX1330 | NWP19 | CONA1_Epitope-1 |
| Erysipelotrichaceae sp., Strain 6_1_45 | NWP181 | PRR27 |
| Klebsiella pneumoniae subsp. pneumoniae, Strain WGLW5 | NWP55 | AVP |
| Lactobacillus jensenii, Strain EX849587VC06 | NWP109 | LECT2 |
| Blautia sp., Strain AAB30 | AB30 | CCL18 |
| Staphylococcus epidermidis, Strain NIH05001 | NWP78 | XG |
| Fusobacterium nucleatum subsp. animalis, Oral Taxon 420, Strain F0419 | NWP196 | TM9SF3 |
| Propionibacterium acnes, Strain HL013PA1 | NWP209 | CNGB3_Epitope-3 |
| Lachnospiraceae bacterium, Strain AAB19 | AB19 | ENDOU |
| Staphylococcus warneri C5 | warneriC5 | CTRB2 |
| Streptococcus sobrinus, Strain W1703 | NWP85 | SSBP3-AS1 |
| Escherichia coli, Strain MS 196-1 | NWP42 | SEMG1 |
| Fusobacterium sp., Strain CM22 | NWP167 | FZD4 |
| Plesiomonas sp., Strain HPP0020 | NWP62 | NPPC |
| Staphylococcus capitis A10 | capitisA10 | AMELX |
| Lachnospiraceae sp., Strain DNF00160 | NWP150 | CXCL9 |
| Escherichia coli, Strain 83972 | NWP04 | LECT2 |
| Staphylococcus warneri B9 | warneriB9 | CDSN |
| Escherichia coli, Strain MS 119-7 | NWP31 | SMOC2 |
| Fusobacterium sp., Strain CM22 | NWP167 | GRM3 |
| Escherichia coli, Strain 83972 | NWP04 | CONA1_Epitope-1 |
| Propionibacterium acnes, Strain HL027PA2 | NWP218 | SLC22A31 |
| Fusobacterium nucleatum subsp. animalis, Oral Taxon 420, Strain F0419 | NWP196 | ENDOU |
| Propionibacterium acnes, Strain HL046PA2 | NWP266 | CNGB3_Epitope-3 |
| Hungatella hathewayi, Strain WAL-18680 | NWP231 | SPINK9 |
| Parabacteroides merdae, Strain CL09T00C40 | NWP184 | MUCL3 |
| Fusobacterium sp., Strain CM22 | NWP153 | ENDOU |
| Ruminococcus gnavus, Strain AAB28 | AB28 | COL10A1 |
| Lactobacillus rhamnosus, Strain LMS2-1 | NWP97 | ENDOU |
| Sporosarcina sp., Strain 2681 | NWP29 | SEMA6C |
| Staphylococcus pasteuri F12 | pasteuriF12 | CDSN |
| Plesiomonas sp., Strain HPP0020 | NWP62 | DEFB130A |
| Staphylococcus pasteuri G7 | pasteuriG7 | TNFRSF21 |
| Prevotella veroralis, Oral Taxon 572, Strain F0319 | NWP155 | CTRB1 |
| Staphylococcus capitis B1 | capitisB1 | CCL2 |
| Klebsiella pneumoniae subsp. pneumoniae, Strain WGLW2 | NWP57 | KISS1 |
| Enterococcus faecalis, Strain TX2137 | NWP117 | PEBP4 |
| Citrobacter freundii, Strain 4_7_47CFAA | NWP28 | PGF |
| Staphylococcus aureus, Strain CM05 | NWP83 | SHISA2 |
| Enterococcus faecium, Strain TX1330 | NWP19 | ADAMTS16 |
| Collinsella sp., Strain 4_8_47FAA | NWP198 | COL10A1 |
| Klebsiella pneumoniae subsp. pneumoniae, Strain WGLW2 | NWP57 | CHGA |
| Staphylococcus capitis A7 | capitisA7 | SFRP4 |
| Clostridium aldenense, Strain WAL-18727 | NWP314 | CCL18 |
| Staphylococcus pasteuri E5 | pasteuriE5 | TNMD |
| Staphylococcus epidermidis, Strain NIHLM020 | NWP74 | TMEM119 |
| Enterococcus faecium, Strain TX0133a04 | NWP44 | PEBP4 |
| Staphylococcus warneri B9 | warneriB9 | CD22 |
| Staphylococcus pasteuri C9 | pasteuriC9 | SFRP4 |
| Escherichia coli, Strain 83972 | NWP04 | SEMG1 |
| Lactobacillus jensenii, Strain EX849587VC06 | NWP109 | TMEM119 |
| Propionibacterium acnes, Strain HL036PA1 | NWP226 | CNGB3_Epitope-3 |
| Staphylococcus lugdunensis F8 | lugdundensisF8 | LOC644613 |
| Lactobacillus crispatus, Strain JV-V01 | NWP107 | PDIA3 |
| Streptococcus downei, Oral Taxon 594, Strain F0415 | NWP123 | PEBP4 |
| Enterococcus faecalis, Strain TX2137 | NWP117 | ASIP |
| Staphylococcus saprophyticus F6 | saprophyticusF6 | ENDOU |
| Staphylococcus warneri D4 | warneriD4 | CTRB2 |
| Staphylococcus hominis B3 | hominisB3 | RTBDN |
| Streptococcus sp., Strain OBRC6 | NWP124 | FAM19A2 |
| Staphylococcus pasteuri F11 | pasteuriF11 | CDSN |
| Prevotella bivia, Strain GED7760C | NWP233 | PF4V1 |
| Shigella sp., Strain D9 | NWP06 | NPPA |
| Lactobacillus gasseri, Strain JV-V03 | NWP106 | NPPC |
| Corynebacterium sp., Strain HFH0082 | NWP61 | CSN3 |
| Fusobacterium nucleatum, Strain CTI-07 | NWP190 | SPINK9 |
| Klebsiella oxytoca, Strain MIT 10-5249 | NWP52 | CD69 |
| Staphylococcus warneri B9 | warneriB9 | EPO |
| Staphylococcus capitis A7 | capitisA7 | OPN3 |
| Corynebacterium sp., Strain HFH0082 | NWP61 | DRAXIN |
| Corynebacterium amycolatum, Strain SK46 | NWP07 | APLP1 |
| Lactobacillus jensenii, Strain 269-3 | NWP110 | LCN2 |
| Bacteroides stercoris, Strain CC31F | NWP270 | SCRG1 |
| Bifidobacterium breve, Strain HPH0326 | NWP289 | SHISA2 |
| Propionibacterium acnes, Strain HL045PA1 | NWP265 | SLC22A31 |
| Staphylococcus epidermidis H6 | EpiH6 | CSN3 |
| Staphylococcus aureus, Strain TCH70 | NWP12 | SHISA2 |
| Klebsiella oxytoca, Strain MIT 10-5246 | NWP50 | VSTM2B |
| Staphylococcus epidermidis C3 | EpiC3 | OTOS |
| Klebsiella sp., Strain 4_1_44FAA | NWP21 | SHISA2 |
| Staphylococcus pasteuri F12 | pasteuriF12 | FAM174A |
| Propionibacterium acnes, Strain HL007PA1 | NWP225 | PDCD1 |
| Escherichia coli, Strain 83972 | NWP04 | DRAXIN |
| Staphylococcus capitis C4 | capitisC4 | NPPA |
| Fusobacterium nucleatum subsp. animalis, Oral Taxon 420, Strain F0419 | NWP196 | GREM2 |
| Staphylococcus warneri D3 | warneriD3 | CTRB2 |
| Lachnospiraceae sp., Strain DNF00160 | NWP150 | FGF17 |
| Staphylococcus epidermidis, Strain NIH04008 | NWP77 | BMP7 |
| Staphylococcus warneri D11 | warneriD12 | SPRN |
| Escherichia coli, Strain MS 110-3 | NWP35 | ADAMTS16 |
| Staphylococcus aureus, Strain MN8 | NWP17 | TMEM119 |
| Lactobacillus jensenii, Strain 269-3 | NWP110 | TFF1 |
| Klebsiella pneumoniae subsp. pneumoniae, Strain WGLW1 | NWP56 | GHRHR |
| Bacteroides fragilis, Strain CL07T12C05 | NWP282 | WNT11 |
| Clostridium aldenense, Strain WAL-18727 | NWP314 | AGRP |
| Micrococcus luteus, Strain SK58 | NWP08 | DRAXIN |
| Proteus mirabilis, Strain WGLW6 | NWP59 | SHISA2 |
| Staphylococcus warneri E6 | warneriE6 | CLEC18A |
| Staphylococcus pasteuri F12 | pasteuriF12 | TMEFF2 |
| Propionibacterium acnes, Strain HL027PA2 | NWP218 | PDCD1 |
| Fusobacterium nucleatum subsp. polymorphum, Strain F0401 | NWP199 | ENDOU |
| Klebsiella pneumoniae subsp. pneumoniae, Strain WGLW1 | NWP56 | NPPC |
| Staphylococcus warneri D11 | warneriD12 | ADRA2C |
| Staphylococcus epidermidis C6 | EpiC6 | LOC644613 |
| Micrococcus luteus, Strain SK58 | NWP08 | CSN3 |
| Fusobacterium nucleatum subsp. animalis, Oral Taxon 420, Strain F0419 | NWP196 | SMOC2 |
| Fusobacterium nucleatum, Strain CTI-07 | NWP190 | SEMG1 |
| Citrobacter freundii, Strain 4_7_47CFAA | NWP88 | TMEM119 |
| Enterococcus faecalis, Strain TUSoD Ef11 | NWP05 | SCNAA_Epitope-3 |
| Micrococcus luteus, Strain SK58 | NWP08 | C4A |
| Staphylococcus haemolytics, Strain DNF00585 | NWP87 | MUCL3 |
| Staphylococcus warneri C2 | warneriC2 | CTRB2 |
| Staphylococcus epidermidis, Strain NIH04008 | NWP77 | SHISA6 |
| Staphylococcus saprophyticus D6 | saprophyticusD6 | BTC |
| Staphylococcus warneri E6 | warneriE6 | PTH1R |
| Streptococcus parasanguinis, Strain F0449 | NWP114 | TMEM119 |
| Klebsiella oxytoca, Strain MIT 09-7231 | NWP45 | GSG1L |
| Eubacterium sp., Strain 3_1_31 | NWP182 | VASN |
| Staphylococcus warneri E6 | warneriE6 | C1orf134 |
| Citrobacter freundii, Strain 4_7_47CFAA | NWP88 | CCL5 |
| Bacteroides sp., Strain 1_1_30 | NWP272 | SLC8B1 |
| Staphylococcus warneri E4 | warneriE4 | EPHA6 |
| Propionibacterium acnes, Strain HL005PA1 | NWP224 | FAM19A4 |
| Staphylococcus hominis, Strain C80 | NWP25 | CCL2 |
| Citrobacter freundii, Strain 4_7_47CFAA | NWP28 | UNQ6190/PRO20217 |
| Streptococcus sp., Strain SR1 | NWP122 | FGF17 |
| Pseudomonas sp., Strain HPB0071 | NWP65 | TMEM119 |
| Staphylococcus epidermidis, Strain W23144 | NWP15 | MUCL3 |
| Citrobacter sp., Strain 30_2 | NWP01 | MANF |
| Bacteroides stercoris, Strain CC31F | NWP270 | IGLL5 |
| Klebsiella oxytoca, Strain MIT 09-7231 | NWP45 | TSPAN9 |
| Staphylococcus warneri A3 | warneriA3 | BTC |
| Enterococcus faecium, Strain TX1330 | NWP19 | ASIP |
| Streptococcus gallolyticus subsp. gallolyticus, Strain TX20005 | NWP27 | TACSTD2 |
| Ruminococcus gnavus, Strain NWP327 | RG151 | CD7 |
| Lactobacillus crispatus, Strain GED7749C | NWP104 | IL17F |
| Bacteroides fragilis, Strain CL05T00C42 | NWP287 | LUM |
| Staphylococcus capitis C4 | capitisC4 | C4A |
| Streptococcus sp., Strain SR1 | NWP122 | CTRB1 |
| Staphylococcus warneri C2 | warneriC2 | SHISA6 |
| Escherichia coli, Strain MS 115-1 | NWP36 | TRPM5 |
| Fusobacterium nucleatum, Strain CTI-03 | NWP187 | EPHA5 |
| Citrobacter freundii, Strain 4_7_47CFAA | NWP28 | TMEM119 |
| Enterococcus faecium, Strain TX1330 | NWP19 | MUC7 |
| Proteus mirabilis, Strain WGLW6 | NWP59 | IL1F10 |
| Bacteroidales bacterium, Strain KA00344 | NWP302 | TMEM149 |
| Streptococcus sp., Strain AS20 | NWP116 | UNQ6190/PRO20217 |
| Streptococcus cristatus, Strain F0329 | NWP126 | SCN1A_Epitope-3 |
| Lactobacillus crispatus, Strain JV-V01 | NWP107 | TNFSF9 |
| Fusobacterium nucleatum, Strain CTI-03 | NWP187 | CCL18 |
| Enterococcus faecium, Strain ERV102 | NWP80 | PRR27 |
| Streptococcus gallolyticus subsp. gallolyticus, Strain TX20005 | NWP27 | SCNAA_Epitope-3 |
| Staphylococcus hominis, Strain C80 | NWP25 | FAM19A3 |
| Citrobacter freundii, Strain 4_7_47CFAA | NWP28 | C1QTNF2 |
| Enterococcus faecium, Strain ERV165 | NWP81 | AGRP |
| Fusobacterium nucleatum, Strain CTI-02 | NWP186 | IGFBP5 |
| Fusobacterium sp., Strain CM22 | NWP167 | LYSMD3 |
| Propionibacterium acnes, Strain HL036PA2 | NWP227 | SLC22A31 |
| Escherichia sp., Strain 3_2_53FAA | NWP02 | DEFB130B |
| Escherichia coli, Strain MS 85-1 | NWP30 | SEMG2 |
| Escherichia coli, Strain MS 69-1 | NWP39 | WISP2 |
| Propionibacterium acnes, Strain HL013PA1 | NWP209 | VSTM2A |
| Bifidobacterium breve, Strain EX336960VC19 | NWP202 | CALCR_Epitope-1 |
| Enterococcus faecalis, Strain S613 | NWP119 | TACSTD2 |
| Arthrobacter albus, Strain DNF00011 | NWP86 | DRAXIN |
| Propionibacterium acnes, Strain SK137 | NWP309 | CNGB3_Epitope-3 |
| Deinococcus grandis, Strain SK125 | NWP89 | IGFBP2 |
| Lachnospiraceae bacterium, Strain AAB23 | AB23 | SYCN |
| Lactobacillus jensenii, Strain 269-3 | NWP110 | SHISA2 |
| Citrobacter sp., Strain 30_2 | NWP01 | SLPI |
| Catabacter hongkongensis, Strain AB8_9 | NWP299 | LYG2 |
| Klebsiella pneumoniae subsp. pneumoniae, Strain WGLW5 | NWP55 | DEFA6 |
| Fusobacterium nucleatum, Strain CTI-06 | NWP189 | MANF |
| Staphylococcus pasteuri E1 | pasteuriE1 | PODXL2 |
| Lachnospiraceae bacterium, Strain AAB19 | AB19 | LOC644613 |
| Staphylococcus pasteuri G7 | pasteuriG7 | CDSN |
| Corynebacterium amycolatum, Strain SK46 | NWP07 | TMEM204 |
| Streptococcus sp., Strain C150 | NWP121 | SHISA6 |
| Fusobacterium nucleatum, Strain CTI-02 | NWP186 | LOC644613 |
| Klebsiella sp., Strain 1_1_55 | NWP03 | PEBP4 |
| Streptococcus downei, Oral Taxon 594, Strain F0415 | NWP123 | FAM19A3 |
| Streptococcus sp., Strain AS20 | NWP116 | PF4V1 |
| Fusobacterium sp., Oral Taxon 370, Strain F0437 | NWP143 | SLC22A31 |
| Proteus mirabilis, Strain WGLW6 | NWP59 | KCT2 |
| Streptococcus sp., Strain AS20 | NWP116 | NPPA |
| Propionibacterium acnes, Strain HL060PA1 | NWP239 | KIR3DL3 |
| Sporosarcina sp., Strain 2681 | NWP29 | FGF19 |
| Enterococcus faecium, Strain ERV102 | NWP80 | DLK1 |
| Staphylococcus warneri D11 | warneriD12 | GPHB5 |
| Escherichia coli, Strain MS 200-1 | NWP41 | C4A |
| Fusobacterium nucleatum, Strain CTI-02 | NWP186 | COL10A1 |
| Streptococcus sp., Strain AS20 | NWP116 | SHISA6 |
| Collinsella aerofaciens, Strain AAB3 | AB3 | FAM19A2 |
| Bacteroides dorei, Strain 5_1_36/D4 | NWP244 | IL10RB |
| Staphylococcus epidermidis H4 | EpiH4 | GZMA |
| Megasphaera micronuciformis, Strain DNF00954 | NWP307 | MUCL3 |
| Lactobacillus gasseri, Strain JV-V03 | NWP106 | CCL18 |
| Staphylococcus epidermidis, Strain NIHLM020 | NWP74 | MUCL3 |
| Streptococcus salivarius, Strain SK126 | NWP118 | FAM19A2 |
| Propionibacterium acnes, Strain HL059PA1 | NWP250 | SPINT3 |
| Klebsiella oxytoca, Strain MIT 10-5246 | NWP50 | TACSTD2 |
| Propionibacterium acnes, Strain HL002PA2 | NWP214 | DSA2D_Epitope-1 |
| Staphylococcus epidermidis, Strain NIHLM001 | NWP66 | CTRB1 |
| Enterococcus faecalis, Strain TX2137 | NWP117 | TFF3 |
| Prevotella bivia, Strain DNF00188 | NWP193 | TMG2_Epitope-1 |
| Propionibacterium acnes, Strain HL050PA1 | NWP248 | CNGB3_Epitope-3 |
| Enterococcus faecium, Strain ERV99 | NWP82 | LOC644613 |
| Dermabacter sp., Strain HFH0086 | NWP92 | SLCO4C1 |
| Escherichia coli, Strain MS 196-1 | NWP42 | CONA1_Epitope-1 |
| Streptococcus sp., Strain SR1 | NWP122 | CLU |
| Staphylococcus aureus, Strain MN8 | NWP17 | EMID1 |
| Escherichia coli, Strain MS 145-7 | NWP33 | APOO |
| Staphylococcus aureus, Strain CM05 | NWP83 | EDAR |
| Staphylococcus warneri D11 | warneriD12 | FGF1 |
| Staphylococcus epidermidis F6 | EpiF6 | CCL18 |
| Collinsella sp., Strain 4_8_47FAA | NWP198 | PIANP |
| Staphylococcus pasteuri E5 | pasteuriE5 | SERPINA11 |
| Staphylococcus saprophyticus E8 | saprophyticusE8 | FGFR1 |
| Prevotella bivia, Strain GED7760C | NWP233 | CXCL9 |
| Sporosarcina sp., Strain 2681 | NWP29 | CSAG1 |
| Propionibacterium acnes, Strain HL002PA3 | NWP219 | CST6 |
| Staphylococcus warneri C2 | warneriC2 | C17orf67 |
| Propionibacterium acnes, Strain HL046PA2 | NWP266 | UNQ9165/PRO28630 |
| Bacteroides stercoris, Strain CC31F | NWP270 | NRN1 |
| Dorea formicigenerans, Strain AAB36 | AB36 | KAL1 |
| Escherichia coli, Strain MS 69-1 | NWP39 | FAM19A2 |
| Clostridium orbiscindens, Strain CC43_001K | NWP259 | ADCY5 |
| Klebsiella pneumoniae subsp. pneumoniae, Strain WGLW3 | NWP54 | AVP |
| Fusobacterium sp., Strain CM21 | NWP152 | MUCL3 |
| Streptococcus sp., Strain ACS2 | NWP115 | APOO |
| Klebsiella pneumoniae subsp. pneumoniae, Strain WGLW5 | NWP55 | TACSTD2 |
| Streptococcus intermedius, Strain F0413 | NWP128 | MUCL3 |
| Plesiomonas sp., Strain HPP0020 | NWP62 | ADAMTS16 |
| Rhodococcus erythropolis, Strain SK121 | NWP95 | FAM19A3 |
| Clostridium cadaveris, Strain CC40_001C | NWP246 | MINPP1 |
| Escherichia coli, Strain MS 200-1 | NWP41 | CONA1_Epitope-1 |
| Fusobacterium sp., Strain CM22 | NWP167 | SDF4 |
| Fusobacterium periodonticum, Strain 1_A_54/D10 | NWP142 | ENDOU |
| Klebsiella oxytoca, Strain MIT 09-7231 | NWP45 | PTGDS |
| Prevotella sp., Oral Taxon 472, Strain F0295 | NWP191 | XG |
| Streptococcus sanguinis, Strain VMC66 | NWP135 | C1QL3 |
| Escherichia coli, Strain MS 145-7 | NWP33 | TMEM149 |
| Propionibacterium acnes, Strain HL043PA2 | NWP264 | SLC22A31 |
| Lactobacillus crispatus, Strain GED7749C | NWP104 | AGER |
| Klebsiella sp., Strain 4_1_44FAA | NWP21 | IFNA17 |
| Lactobacillus jensenii, Strain 269-3 | NWP110 | TMEM59L |
| Streptococcus mitis, Strain F0392 | NWP26 | RTBDN |
| Staphylococcus warneri F1 | warneriF1 | SSBP3-AS1 |
| Streptococcus sobrinus, Strain W1703 | NWP85 | PEBP4 |
| Plesiomonas sp., Strain HPP0020 | NWP62 | CHRDL2 |
| Enterococcus faecium, Strain E417 | NWP79 | ASIP |
| Escherichia coli, Strain MS 115-1 | NWP36 | NTRK1 |
| Streptococcus sp., Strain CM7 | NWP133 | LOC644613 |
| Streptococcus cristatus, Strain F0329 | NWP126 | TMEM119 |
| Escherichia coli, Strain MS 115-1 | NWP36 | CONA1_Epitope-1 |
| Streptococcus sp., Strain ACC21 | NWP134 | CTRB1 |
| Klebsiella oxytoca, Strain MIT 10-5246 | NWP50 | PTGDS |
| Megasphaera sp., Strain DNF00872 | NWP295 | PDIA6 |
| Lachnospiraceae bacterium, Strain AAB26 | AB26 | CC50C_Epitope-1 |
| Klebsiella sp., Strain 1_1_55 | NWP03 | DQA2_Epitope-1 |
| Propionibacterium acnes, Strain HL027PA2 | NWP218 | SPINK14 |
| Lactobacillus jensenii, Strain EX849587VC03 | NWP108 | GPR17 |
| Fusobacterium sp., Oral Taxon 370, Strain F0437 | NWP143 | TUSC5 |
| Veillonella sp., Oral Taxon 780, Strain F0422 | NWP268 | NGFR |
| Escherichia coli, Strain MS 85-1 | NWP30 | LECT2 |
| Capnocytophaga sputigena, Strain CC21_001D | NWP163 | AMY2A |
| Streptococcus sp., Strain AS20 | NWP116 | CHRDL2 |
| Oscillibacter sp., Strain KLE-1728 | NWP269 | CCL18 |
| Blautia sp., Strain KLE 1732 | NWP292 | PLA2G2E |
| Fusobacterium sp., Oral Taxon 370, Strain F0437 | NWP143 | TREML1 |
| Escherichia coli, Strain MS 145-7 | NWP33 | DNASE1L2 |
| Staphylococcus hominis, Strain SK119 | NWP10 | TMEM119 |
| Corynebacterium sp., Strain CMW7794 | NWP98 | RCN1 |
| Catabacter hongkongensis, Strain AB8_9 | NWP299 | PRR27 |
| Staphylococcus warneri E4 | warneriE4 | ECE1 |
| Corynebacterium sp., Strain CMW7794 | NWP98 | DEFB130A |
| Propionibacterium acnes, Strain HL020PA1 | NWP221 | PDCD1 |
| Staphylococcus capitis C4 | capitisC4 | DEFB130A |
| Allobaculum sp., Strain NWP324 | Ery128 | SLC2A2 |
| Streptococcus mitis, Strain F0392 | NWP26 | SEMG2 |
| Streptococcus sp., Strain SR1 | NWP122 | LOC644613 |
| Propionibacterium acnes, Strain HL005PA1 | NWP224 | PDCD1 |
| Staphylococcus capitis C4 | capitisC4 | LECT2 |
| Fusobacterium periodonticum, Strain 2_1_31 | NWP160 | PTGDS |
| Streptococcus anginosus, Strain F0211 | NWP125 | FAM19A2 |
| Staphylococcus pasteuri G7 | pasteuriG7 | TNMD |
| Prevotella buccae, Strain D17 | NWP256 | SLC22A14 |
| Prevotella bivia, Strain DNF00188 | NWP193 | CXCL9 |
| Streptococcus anginosus, Strain F0211 | NWP125 | CSAG1 |
| Rothia mucilaginosa, Strain CC87LB | NWP84 | NETO1 |
| Staphylococcus warneri D11 | warneriD12 | CLEC18A |
| Enterococcus faecium, Strain ERV165 | NWP81 | CONA1_Epitope-1 |
| Corynebacterium sp., Strain CMW7794 | NWP98 | NPPA |
| Staphylococcus xylosus F2 | xylosusF2 | CCL2 |
| Staphylococcus epidermidis C3 | EpiC3 | C2orf40 |
| Bacteroides stercoris, Strain CC31F | NWP270 | IFNA6 |
| Staphylococcus haemolyticus G9 | haemolyticusG9 | SLC8B1 |
| Klebsiella oxytoca, Strain MIT 10-5249 | NWP52 | GP6 |
| Escherichia coli, Strain MS 16-3 | NWP37 | TMEM119 |
| Staphylococcus gallinarum E9 | gallinarumE9 | FAM3C |
| Staphylococcus pasteuri F11 | pasteuriF11 | TNMD |
| Staphylococcus pasteuri G3 | pasteuriG3 | CDSN |
| Escherichia coli, Strain MS 115-1 | NWP36 | SEMG2 |
| Escherichia coli, Strain MS 57-2 | NWP34 | SDC3 |
| Clostridiales bacterium, Strain 4_1_37FAA | NWP285 | PDIA6 |
| Staphylococcus warneri D9 | warneriD9 | CTRB2 |
| Corynebacterium amycolatum, Strain SK46 | NWP07 | PROK1 |
| Streptococcus salivarius, Strain SK126 | NWP118 | CTRB1 |
| Enterococcus faecalis, Strain TUSoD Ef11 | NWP05 | LOC644613 |
| Propionibacterium acnes, Strain HL002PA2 | NWP214 | SLC22A31 |
| Fusobacterium sp., Strain CM22 | NWP167 | LCN2 |
| Megasphaera micronuciformis, Strain DNF00954 | NWP307 | PLA2G2E |
| Fusobacterium nucleatum, Strain CTI-03 | NWP187 | LOC644613 |
| Enterococcus faecalis, Strain TX2137 | NWP117 | INSL4 |
| Staphylococcus capitis E12 | capitisE12 | SSBP3-AS1 |
| Streptococcus gallolyticus subsp. gallolyticus, Strain TX20005 | NWP27 | IGSF4B |
| Klebsiella pneumoniae subsp. pneumoniae, Strain WGLW2 | NWP57 | AVP |
| Escherichia coli, Strain MS 110-3 | NWP35 | TRPM5 |
| Staphylococcus warneri B9 | warneriB9 | EPHB3 |
| Streptococcus gallolyticus subsp. gallolyticus, Strain TX20005 | NWP27 | SLC8B1 |
| Klebsiella oxytoca, Strain MIT 10-5249 | NWP52 | C5orf64 |
| Escherichia coli, Strain MS 85-1 | NWP30 | RCN1 |
| Staphylococcus epidermidis, Strain NIH04008 | NWP77 | CCL17 |
| Enterococcus faecium, Strain TX1330 | NWP19 | LECT2 |
| Lactobacillus crispatus, Strain JV-V01 | NWP107 | GPR25 |
| Staphylococcus capitis A7 | capitisA7 | AT134_Epitope-2 |
| Propionibacterium acnes, Strain HL001PA1 | NWP203 | SDC1 |
| Escherichia coli, Strain MS 110-3 | NWP35 | SEMG2 |
| Lactobacillus crispatus, Strain JV-V01 | NWP107 | MANF |
| Escherichia coli, Strain 83972 | NWP04 | AGRP |
| Bifidobacterium angulatum, Strain F16_22 | NWP291 | EPHA5 |
| Propionibacterium acnes, Strain HL036PA2 | NWP227 | TMEM59L |
| Erysipelotrichaceae sp., Strain 6_1_45 | NWP181 | CONA1_Epitope-1 |
| Dorea formicigenerans, Strain AAB36 | AB36 | CTRB2 |
| Proteus mirabilis, Strain WGLW6 | NWP59 | TTYH1_Epitope-3 |
| Propionibacterium acnes, Strain HL037PA2 | NWP205 | DAG1 |
| Bacteroides fragilis, Strain 3_1_12 | NWP271 | LRFN1 |
| Corynebacterium sp., Strain HFH0082 | NWP61 | CSAG1 |
| Streptococcus sp., Strain OBRC6 | NWP124 | UNQ6190/PRO20217 |
| Staphylococcus pasteuri E5 | pasteuriE5 | FAT2 |
| Propionibacterium acnes, Strain HL060PA1 | NWP239 | GYPA |
| Klebsiella oxytoca, Strain MIT 09-7231 | NWP45 | SCN1A_Epitope-3 |
| Lactobacillus jensenii, Strain 269-3 | NWP110 | TNFRSF4 |
| Allobaculum sp., Strain NWP324 | Ery128 | SLC6A13 |
| Fusobacterium sp., Strain CM22 | NWP167 | RCN1 |
| Klebsiella oxytoca, Strain MIT 10-5243 | NWP47 | CHGA |
| Fusobacterium periodonticum, Strain 2_1_31 | NWP160 | KCNK1 |
| Staphylococcus capitis, Strain SK14 | NWP09 | GPR151 |
| Enterococcus faecium, Strain ERV165 | NWP81 | IGFBP5 |
| Klebsiella oxytoca, Strain MIT 10-5246 | NWP50 | FAM174A |
| Streptococcus vestibularis, Strain F0396 | NWP132 | PIANP |
| Staphylococcus capitis A7 | capitisA7 | PSG4 |
| Enterococcus faecium, Strain TX0133a04 | NWP44 | PROK1 |
| Megasphaera sp., Strain DNF00872 | NWP295 | TNFSF9 |
| Dermabacter sp., Strain HFH0086 | NWP92 | SLC22A31 |
| Prevotella buccae, Strain D17 | NWP256 | PF4V1 |
| Propionibacterium acnes, Strain HL060PA1 | NWP239 | CXCR7 |
| Bacillus sp., Strain DNF00233 | NWP90 | C2orf40 |
| Staphylococcus epidermidis, Strain NIHLM040 | NWP76 | SMR3A |
| Enterococcus faecium, Strain TX1330 | NWP19 | KLK10 |
| Rothia aeria, Oral Taxon 188, Strain F0474 | NWP64 | TRABD2B |
| Bacteroidales bacterium, Strain KA00344 | NWP302 | CD14 |
| Staphylococcus warneri, Strain SK66 | NWP11 | CAC1I_Epitope-12 |
| Dorea formicigenerans, Strain AAB36 | AB36 | CCL13 |
| Enterococcus faecium, Strain E417 | NWP79 | LYVE1 |
| Staphylococcus warneri A8 | warneriA8 | SLC8B1 |
| Lactobacillus crispatus, Strain JV-V01 | NWP107 | RLN2 |
| Streptococcus anginosus, Strain F0211 | NWP125 | SHISA6 |
| Bacillus sp., Strain DNF00233 | NWP90 | SPINK9 |
| Staphylococcus epidermidis, Strain NIHLM040 | NWP76 | CLU |
| Escherichia coli, Strain MS 16-3 | NWP37 | PRR27 |
| Fusobacterium nucleatum, Strain CTI-02 | NWP186 | FGFRL1 |
| Streptococcus salivarius, Strain SK126 | NWP118 | TMEM119 |
| Staphylococcus capitis A5 | capitisA5 | LOC644613 |
| Peptoniphilus lacrimalis, Strain UPII 315-B | NWP261 | SLC8B1 |
| Eggerthella sp., Strain MVA1 | NWP185 | DEFB130B |
| Corynebacterium sp., Strain HFH0082 | NWP61 | C2orf66 |
| Erysipelotrichaceae sp., Strain 6_1_45 | NWP174 | PRR27 |
| Deinococcus grandis, Strain SK125 | NWP89 | NPPC |
| Erysipelotrichaceae sp., Strain 6_1_45 | NWP181 | CSAG1 |
| Klebsiella sp., Strain 4_1_44FAA | NWP21 | DEFA6 |
| Propionibacterium acnes, Strain HL053PA1 | NWP260 | CNGB3_Epitope-3 |
| Erysipelotrichaceae sp., Strain 6_1_45 | NWP174 | DEFB130A |
| Staphylococcus cohnii G11 | cohniiG11 | SO1C1_Epitope-5 |
| Staphylococcus capitis C12 | capitisC12 | FAM19A3 |
| Fusobacterium nucleatum subsp. animalis, Strain D11 | NWP141 | PF4V1 |
| Staphylococcus epidermidis F6 | EpiF6 | ADAMTS16 |
| Streptococcus mitis, Strain F0392 | NWP26 | CSAG1 |
| Bifidobacterium longum subsp. longum, Strain 44B | NWP308 | PRR27 |
| Fusobacterium nucleatum, Strain CTI-03 | NWP187 | IGFBP5 |
| Hungatella hathewayi, Strain WAL-18680 | NWP231 | TFF1 |
| Peptoniphilus lacrimalis, Strain DNF00528 | NWP304 | CD14 |
| Sporosarcina sp., Strain 2681 | NWP29 | ASIP |
| Klebsiella sp., Strain 1_1_55 | NWP03 | PPY |
| Erysipelotrichaceae sp., Strain 6_1_45 | NWP181 | KLK10 |
| Proteus mirabilis, Strain WGLW6 | NWP59 | IFNGR1 |
| Fusobacterium sp., Strain CM21 | NWP152 | CTRB1 |
| Lactobacillus gasseri, Strain JV-V03 | NWP106 | ASIP |
| Corynebacterium sp., Strain HFH0082 | NWP61 | RCN1 |
| Staphylococcus epidermidis H4 | EpiH4 | LOC644613 |
| Propionibacterium acnes, Strain HL030PA2 | NWP207 | CNGB3_Epitope-3 |
| Micrococcus luteus, Strain SK58 | NWP08 | MUCL3 |
| Streptococcus gallolyticus subsp. gallolyticus, Strain TX20005 | NWP27 | MYPR_Epitope-2 |
| Lactobacillus jensenii, Strain EX849587VC06 | NWP109 | CSN3 |
| Klebsiella oxytoca, Strain MIT 10-5243 | NWP47 | DQA2_Epitope-1 |
| Fusobacterium nucleatum subsp. animalis, Oral Taxon 420, Strain F0419 | NWP196 | DEFB115 |
| Streptococcus parasanguinis, Strain F0449 | NWP114 | RTBDN |
| Eubacterium infirmum, Strain F0142 | NWP223 | TXNDC12 |
| Weissella cibaria, Strain F16_1 | NWP101 | CTRB1 |
| Staphylococcus capitis A7 | capitisA7 | F3 |
| Plesiomonas sp., Strain HPP0020 | NWP62 | SEMG1 |
| Propionibacterium acnes, Strain HL005PA1 | NWP224 | SLC22A31 |
| Lactobacillus jensenii, Strain EX849587VC06 | NWP109 | NPPC |
| Staphylococcus warneri, Strain SK66 | NWP11 | CONA1_Epitope-1 |
| Klebsiella pneumoniae subsp. pneumoniae, Strain WGLW3 | NWP54 | DEFB130B |
| Micrococcus luteus, Strain SK58 | NWP08 | PF4V1 |
| Ruminococcus gnavus, Strain NWP326 | RG109 | SPINK9 |
| Propionibacterium acnes, Strain HL013PA2 | NWP217 | LAYN |
| Escherichia coli, Strain MS 16-3 | NWP37 | DEFB130A |
| Propionibacterium acnes, Strain HL002PA3 | NWP219 | PDCD1 |
| Streptococcus salivarius, Strain SK126 | NWP118 | TFF3 |
| Propionibacterium acnes, Strain HL030PA1 | NWP212 | TMEM119 |
| Fusobacterium sp., Strain CM22 | NWP167 | EDDM3B |
| Peptoniphilus sp., Oral Taxon 836, Strain F0141 | NWP313 | PGLYRP1 |
| Escherichia coli, Strain 83972 | NWP04 | DEFB130A |
| Staphylococcus capitis C11 | capitisC11 | P4HB |
| Staphylococcus aureus, Strain TCH70 | NWP12 | EDAR |
| Enterococcus faecium, Strain E417 | NWP79 | DEFB130B |
| Bacillus sp., Strain DNF00233 | NWP90 | DEFB130A |
| Staphylococcus capitis A7 | capitisA7 | OR1L1_Epitope-1 |
| Lactobacillus gasseri, Strain EX33690VC16 | NWP113 | AGER |
| Proteus mirabilis, Strain WGLW6 | NWP59 | MCFD2 |
| Streptococcus sp., Strain SR1 | NWP122 | BTC |
| Streptococcus sobrinus, Strain W1703 | NWP85 | GPR17 |
| Staphylococcus hominis B3 | hominisB3 | CCL17 |
| Bifidobacterium breve, Strain EX336960VC19 | NWP202 | CD300C |
| Proteus mirabilis, Strain WGLW4 | NWP58 | OPN4 |
| Collinsella sp., Strain 4_8_47FAA | NWP198 | CSAG1 |
| Klebsiella pneumoniae subsp. pneumoniae, Strain WGLW5 | NWP55 | VSTM2B |
| Streptococcus gallolyticus subsp. gallolyticus, Strain TX20005 | NWP27 | CHGA |
| Bacteroides stercoris, Strain CC31F | NWP270 | BST2 |
| Escherichia sp., Strain 3_2_53FAA | NWP02 | ANO8_Epitope-1 |
| Staphylococcus capitis A7 | capitisA7 | GPHB5 |
| Enterococcus faecalis, Strain TX2137 | NWP117 | FAM3A |
| Prevotella histicola, Strain F0411 | NWP144 | TMCO3 |
| Propionibacterium acnes, Strain HL002PA3 | NWP219 | SLC22A31 |
| Staphylococcus hominis, Strain SK119 | NWP10 | NAPSA |
| Staphylococcus aureus, Strain CM05 | NWP83 | C4orf48 |
| Enterococcus faecium, Strain ERV99 | NWP82 | PDGFB |
| Staphylococcus haemolytics, Strain DNF00585 | NWP87 | CCL2 |
| Fusobacterium sp., Strain CM22 | NWP167 | SRGN |
| Fusobacterium nucleatum, Strain CTI-03 | NWP187 | NPPC |
| Propionibacterium acnes, Strain HL036PA2 | NWP227 | TGFA |
| Enterococcus faecium, Strain ERV102 | NWP80 | CCL17 |
| Propionibacterium acnes, Strain HL002PA2 | NWP214 | PDCD1 |
| Propionibacterium acnes, Strain HL020PA1 | NWP221 | CNGB3_Epitope-3 |
| Veillonella sp., Oral Taxon 780, Strain F0422 | NWP268 | PDGFB |
| Propionibacterium acnes, Strain HL038PA1 | NWP235 | CST6 |
| Shigella sp., Strain D9 | NWP06 | TACSTD2 |
| Klebsiella oxytoca, Strain MIT 10-5249 | NWP52 | CXCR5 |
| Oscillibacter sp., Strain KLE 1745 | NWP252 | NGFR |
| Streptococcus sp., Strain SR1 | NWP122 | SHISA6 |
| Propionibacterium acnes, Strain HL036PA1 | NWP226 | UNQ9165/PRO28630 |
| Staphylococcus hominis B3 | hominisB3 | CTRB1 |
| Escherichia coli, Strain MS 85-1 | NWP30 | DRAXIN |
| Streptococcus gallolyticus subsp. gallolyticus, Strain TX20005 | NWP27 | SHISA2 |
| Escherichia coli, Strain MS 145-7 | NWP33 | TMEM119 |
| Staphylococcus warneri D11 | warneriD12 | C1orf134 |
| Fusobacterium nucleatum, Strain CTI-05 | NWP188 | MUC7 |
| Lactobacillus jensenii, Strain 269-3 | NWP110 | FCGR3B |
| Escherichia coli, Strain MS 119-7 | NWP31 | DEFA6 |
| Enterococcus faecalis, Strain R712 | NWP120 | TACSTD2 |
| Bacteroides sp., Strain 4_1_36 | NWP312 | PLA2G2E |
| Clostridium orbiscindens, Strain CC43_001K | NWP259 | CFP |
| Staphylococcus epidermidis, Strain NIHLM031 | NWP73 | TMEM119 |
| Staphylococcus epidermidis, Strain NIHLM040 | NWP76 | CLCC1 |
| Escherichia coli, Strain MS 16-3 | NWP37 | NPPC |
| Klebsiella oxytoca, Strain MIT 10-5245 | NWP49 | AVP |
| Peptoniphilus sp., Oral Taxon 836, Strain F0141 | NWP313 | PLA2G2E |
| Staphylococcus pasteuri G7 | pasteuriG7 | SERPINA11 |
| Fusobacterium nucleatum, Strain CTI-05 | NWP188 | NPPC |
| Corynebacterium sp., Strain HFH0082 | NWP61 | PROK1 |
| Eggerthella sp., Strain MVA1 | NWP185 | SEMG1 |
| Prevotella denticola, Strain DNF00960 | NWP179 | LOC644613 |
| Fusobacterium sp., Strain CM21 | NWP152 | EPHA5 |
| Enterococcus faecium, Strain TX1330 | NWP19 | CSAG1 |
| Lactobacillus jensenii, Strain 208-1 | NWP111 | PF4V1 |
| Enterococcus faecium, Strain ERV102 | NWP80 | DEFB130A |
| Streptococcus sp., Strain BS29a | NWP129 | SHISA6 |
| Streptococcus downei, Oral Taxon 594, Strain F0415 | NWP123 | FAM19A5 |
| Staphylococcus warneri B9 | warneriB9 | FAM174A |
| Staphylococcus warneri E4 | warneriE4 | CSF2RA |
| Staphylococcus epidermidis, Strain NIH04008 | NWP77 | CTSL |
| Lactobacillus jensenii, Strain 269-3 | NWP110 | CSPG5 |
| Bacteroides sp., Strain 1_1_30 | NWP272 | CHGA |
| Klebsiella oxytoca, Strain MIT 10-5243 | NWP47 | VSTM2B |
| Bacteroides stercoris, Strain CC31F | NWP270 | KCNK4_Epitope-1 |
| Erysipelotrichaceae sp., Strain 6_1_45 | NWP174 | CCL22 |
| Fusobacterium sp., Oral Taxon 370, Strain F0437 | NWP143 | P2RY13 |
| Prevotella denticola, Strain DNF00960 | NWP179 | IER3 |
| Bacillus sp., Strain DNF00233 | NWP90 | TMEM204 |
| Streptococcus sp., Strain AS20 | NWP116 | DEFB130A |
| Fusobacterium sp., Strain CM21 | NWP166 | MUC7 |
| Staphylococcus epidermidis H4 | EpiH4 | CCL2 |
| Klebsiella oxytoca, Strain MIT 09-7231 | NWP45 | TMEM9B |
| Klebsiella pneumoniae subsp. pneumoniae, Strain WGLW1 | NWP56 | DEFB130B |
| Proteus mirabilis, Strain WGLW6 | NWP59 | CD248 |
| Bacteroides sp., Strain 9_1_42FAA | NWP274 | NPDC1 |
| Staphylococcus epidermidis, Strain NIHLM001 | NWP66 | SHISA6 |
| Staphylococcus warneri, Strain SK66 | NWP11 | TRPV1 |
| Proteus mirabilis, Strain WGLW4 | NWP58 | KCNK4_Epitope-1 |
| Arthrobacter albus, Strain DNF00011 | NWP86 | FAM174A |
| Escherichia coli, Strain MS 85-1 | NWP30 | PRND |
| Streptococcus sp., Strain ACS2 | NWP115 | NPDC1 |
| Staphylococcus warneri, Strain SK66 | NWP11 | MMP24 |
| Escherichia coli, Strain MS 85-1 | NWP30 | C4A |
| Streptococcus salivarius, Strain SK126 | NWP118 | FAM19A5 |
| Klebsiella sp., Strain MS 92-3 | NWP40 | AVP |
| Lactobacillus crispatus, Strain JV-V01 | NWP107 | SGCA |
| Lachnospiraceae bacterium, Strain AAB19 | AB19 | AMELX |
| Klebsiella pneumoniae subsp. pneumoniae, Strain WGLW1 | NWP56 | PIANP |
| Staphylococcus warneri, Strain SK66 | NWP11 | PTPRG |
| Escherichia coli, Strain MS 196-1 | NWP42 | TMEM119 |
| Streptococcus anginosus, Strain F0211 | NWP125 | PIANP |
| Streptococcus salivarius, Strain SK126 | NWP118 | SHISA6 |
| Fusobacterium nucleatum subsp. animalis, Oral Taxon 420, Strain F0419 | NWP196 | CLU |
| Escherichia coli, Strain MS 200-1 | NWP41 | DRAXIN |
| Weissella cibaria, Strain F16_1 | NWP101 | CSAG1 |
| Klebsiella pneumoniae subsp. pneumoniae, Strain WGLW3 | NWP54 | PLA2G2E |
| Bacillus sp., Strain DNF00233 | NWP90 | RARRES2 |
| Citrobacter sp., Strain 30_2 | NWP01 | INSL4 |
| Staphylococcus warneri D11 | warneriD12 | MTNR1B |
| Streptococcus downei, Oral Taxon 594, Strain F0415 | NWP123 | SOSTDC1 |
| Enterococcus faecium, Strain E417 | NWP79 | PLA2G2E |
| Streptococcus sp., Strain AS20 | NWP116 | SEMG2 |
| Streptococcus sobrinus, Strain W1703 | NWP85 | ASIP |
| Fusobacterium sp., Oral Taxon 370, Strain F0437 | NWP143 | MCAM |
| Pseudomonas sp., Strain HPB0071 | NWP65 | NCKX4_Epitope-1 |
| Streptococcus sp., Strain C150 | NWP121 | ASIP |
| Staphylococcus capitis A7 | capitisA7 | C1orf134 |
| Propionibacterium acnes, Strain HL005PA1 | NWP224 | CST6 |
| Escherichia coli, Strain MS 115-1 | NWP36 | MUCL3 |
| Propionibacterium acnes, Strain HL060PA1 | NWP239 | ITPR2_Epitope-3 |
| Staphylococcus capitis D5 | capitisD5 | SGCA |
| Propionibacterium acnes, Strain HL013PA1 | NWP209 | SLC6A13 |
| Propionibacterium acnes, Strain HL027PA1 | NWP211 | OPRK1 |
| Plesiomonas sp., Strain HPP0020 | NWP62 | IGFBP5 |
| Streptococcus sp., Strain SR1 | NWP122 | DRAXIN |
| Bacteroides salyersiae, Strain CL02T12C01 | NWP276 | AVP |
| Citrobacter freundii, Strain 4_7_47CFAA | NWP28 | FGFRL1 |
| Enterococcus faecalis, Strain TX2137 | NWP117 | TACSTD2 |
| Bacillus sp., Strain DNF00233 | NWP90 | GPR15L |
| Weissella cibaria, Strain F16_1 | NWP101 | PIANP |
| Fusobacterium nucleatum, Strain CTI-03 | NWP187 | PDIA6 |
| Prevotella bivia, Strain DNF00650 | NWP176 | FGF17 |
| Escherichia coli, Strain MS 110-3 | NWP35 | DRAXIN |
| Propionibacterium acnes, Strain HL030PA1 | NWP212 | OPRK1 |
| Propionibacterium acnes, Strain HL044PA1 | NWP237 | OPRK1 |
| Veillonella sp., Oral Taxon 780, Strain F0422 | NWP268 | P4HB |
| Bacteroides sp., Strain AAB9 | AB9 | EPHB2 |
| Propionibacterium acnes, Strain HL043PA2 | NWP264 | PDCD1 |
| Escherichia coli, Strain MS 115-1 | NWP36 | C4A |
| Bacteroides sp., Strain AAB9 | AB9 | PTPRJ |
| Propionibacterium acnes, Strain HL036PA1 | NWP226 | PDCD1 |
| Streptococcus mitis, Strain F0392 | NWP26 | FAM19A2 |
| Streptococcus salivarius, Strain SK126 | NWP118 | BTC |
| Staphylococcus epidermidis, Strain E13A | NWP63 | HGFAC |
| Propionibacterium acnes, Strain HL044PA1 | NWP237 | TMEM119 |
| Staphylococcus pasteuri E5 | pasteuriE5 | TMEFF2 |
| Erysipelotrichaceae sp., Strain 6_1_45 | NWP181 | AGRP |
| Enterococcus faecalis, Strain S613 | NWP119 | SCNAA_Epitope-3 |
| Staphylococcus warneri, Strain SK66 | NWP11 | SL9A1_Epitope-1 |
| Klebsiella oxytoca, Strain MIT 10-5250 | NWP53 | AVP |
| Staphylococcus capitis A7 | capitisA7 | CSMT1_Epitope-1 |
| Escherichia coli, Strain MS 145-7 | NWP33 | QRFP |
| Streptococcus sp., Strain AS20 | NWP116 | SEMG1 |
| Staphylococcus pasteuri G3 | pasteuriG3 | CSPG5 |
| Streptococcus sp., Strain SR1 | NWP122 | AGER |
| Eubacterium sp., Strain 3_1_31 | NWP182 | FGF17 |
| Staphylococcus saprophyticus F6 | saprophyticusF6 | FAM3C |
| Klebsiella oxytoca, Strain MIT 09-7231 | NWP45 | COL26A1 |
| Staphylococcus pasteuri E5 | pasteuriE5 | TNFRSF21 |
| Propionibacterium acnes, Strain HL036PA3 | NWP222 | SLC22A31 |
| Lactobacillus jensenii, Strain EX849587VC03 | NWP108 | TFF1 |
| Escherichia coli, Strain MS 69-1 | NWP39 | TMEM204 |
| Staphylococcus aureus, Strain TCH70 | NWP12 | SO1B7_Epitope-5 |
| Enterococcus faecium, Strain TX0133a04 | NWP44 | FGF17 |
| Oscillibacter sp., Strain KLE-1728 | NWP269 | GPR25 |
| Staphylococcus capitis B12 | capitisB12 | BMPR1B |
| Escherichia coli, Strain MS 57-2 | NWP34 | MCAM |
| Rothia mucilaginosa, Strain CC87LB | NWP84 | CTRB1 |
| Staphylococcus capitis A7 | capitisA7 | TMPRSS11D |
| Lactobacillus crispatus, Strain JV-V01 | NWP107 | SHISA2 |
| Streptococcus sp., Strain AS20 | NWP116 | CCL18 |
| Staphylococcus epidermidis G4 | EpiG4 | MUCL3 |
| Staphylococcus capitis B8 | capitisB8 | CCL18 |
| Enterococcus faecium, Strain TX1330 | NWP19 | RCN1 |
| Streptococcus salivarius, Strain SK126 | NWP118 | CSAG1 |
| Plesiomonas sp., Strain HPP0020 | NWP62 | APLP1 |
| Allobaculum sp., Strain NWP324 | Ery128 | NPY4R |
| Eggerthella sp., Strain MVA1 | NWP185 | GNLY |
| Clostridium sp., Strain MSTE9 | NWP253 | DRAXIN |
| Escherichia coli, Strain MS 57-2 | NWP34 | TMEM119 |
| Propionibacterium acnes, Strain HL060PA1 | NWP239 | C5orf64 |
| Deinococcus grandis, Strain SK125 | NWP89 | LOC644613 |
| Bifidobacterium sp., Strain MSTE12 | NWP242 | IFNA17 |
| Blautia sp., Strain AAB30 | AB30 | LOC644613 |
| Bacteroides stercoris, Strain CC31F | NWP270 | RBP4 |
| Proteus mirabilis, Strain WGLW6 | NWP59 | C6orf120 |
| Propionibacterium acnes, Strain HL001PA1 | NWP203 | S12A6_Epitope-3 |
| Escherichia coli, Strain MS 57-2 | NWP34 | ALPP |
| Lachnospiraceae bacterium, Strain AAB19 | AB19 | AGRP |
| Escherichia sp., Strain 3_2_53FAA | NWP02 | NPPC |
| Clostridiales bacterium, Strain 4_1_37FAA | NWP285 | PDIA2 |
| Selenomonas noxia, Strain F0398 | NWP213 | PRR27 |
| Corynebacterium amycolatum, Strain SK46 | NWP07 | CONA1_Epitope-1 |
| Ruminococcus gnavus, Strain NWP325 | RG74 | PDIA6 |
| Citrobacter sp., Strain 30_2 | NWP01 | BTC |
| Propionibacterium acnes, Strain HL050PA3 | NWP249 | SPINT3 |
| Micrococcus luteus, Strain SK58 | NWP08 | DMKN |
| Corynebacterium amycolatum, Strain SK46 | NWP07 | RCN1 |
| Arthrobacter albus, Strain DNF00011 | NWP86 | CRLF1 |
| Blautia sp., Strain AAB30 | AB30 | CSN3 |
| Streptococcus salivarius, Strain SK126 | NWP118 | SYCN |
| Proteus mirabilis, Strain WGLW4 | NWP58 | ZACN |
| Prevotella histicola, Strain F0411 | NWP144 | PDIA3 |
| Propionibacterium acnes, Strain HL037PA1 | NWP208 | GPR37 |
| Eggerthella sp., Strain MVA1 | NWP185 | DEFB130A |
| Corynebacterium sp., Strain HFH0082 | NWP61 | SEMA6C |
| Propionibacterium sp., Strain 5_U_42AFAA | NWP300 | SLC22A31 |
| Megasphaera micronuciformis, Strain DNF00954 | NWP307 | FAM19A3 |
| Veillonella sp., Oral Taxon 780, Strain F0422 | NWP268 | CXCR4 |
| Bifidobacterium longum subsp. longum, Strain 44B | NWP308 | NPPC |
| Streptococcus gallolyticus subsp. gallolyticus, Strain TX20005 | NWP27 | LGALS7 |
| Acidaminococcus sp., Strain HPA0509 | NWP175 | DEFB130B |
| Klebsiella sp., Strain MS 92-3 | NWP40 | DEFA6 |
| Staphylococcus warneri D9 | warneriD9 | LOC644613 |
| Staphylococcus warneri B9 | warneriB9 | SPACA6P |
| Fusobacterium sp., Strain CM22 | NWP167 | EPHA4 |
| Escherichia coli, Strain MS 200-1 | NWP41 | COL10A1 |
| Fusobacterium nucleatum, Strain CTI-06 | NWP189 | CTRB1 |
| Escherichia coli, Strain 83972 | NWP04 | FAM19A2 |
| Proteus mirabilis, Strain WGLW6 | NWP59 | SO1A2_Epitope-5 |
| Klebsiella oxytoca, Strain MIT 10-5249 | NWP52 | TMEM149 |
| Prevotella timonensis, Strain CRIS 5C-B1 | NWP156 | P4HB |
| Bacteroides sp., Strain D2 | NWP192 | NPPC |
| Propionibacterium acnes, Strain HL005PA2 | NWP215 | SLC22A31 |
| Lactobacillus gasseri, Strain JV-V03 | NWP106 | CCL17 |
| Citrobacter sp., Strain 30_2 | NWP01 | PGF |
| Streptococcus sobrinus, Strain W1703 | NWP85 | FGFRL1 |
| Lactobacillus jensenii, Strain EX849587VC03 | NWP108 | CAC1I_Epitope-9 |
| Propionibacterium acnes, Strain HL036PA3 | NWP222 | PDCD1 |
| Citrobacter sp., Strain 30_2 | NWP01 | DKK1 |
| Bacteroides sp., Strain 1_1_30 | NWP272 | PF4V1 |
| Blautia sp., Strain AAB30 | AB30 | AGRP |
| Peptoniphilus sp., Strain CMW7756A | NWP162 | AMY2A |
| Lactobacillus gasseri, Strain EX33690VC16 | NWP113 | TFF2 |
| Sporosarcina sp., Strain 2681 | NWP29 | RARRES2 |
| Propionibacterium acnes, Strain HL005PA2 | NWP215 | TGFA |
| Erysipelotrichaceae sp., Strain 6_1_45 | NWP174 | RARRES2 |
| Bacillus sp., Strain DNF00233 | NWP90 | FAM19A3 |
| Staphylococcus epidermidis F6 | EpiF6 | FAM19A3 |
| Propionibacterium acnes, Strain HL037PA2 | NWP205 | GNRH2 |
| Staphylococcus capitis B8 | capitisB8 | KLK10 |
| Corynebacterium sp., Strain CMW7794 | NWP98 | DEFB115 |
| Staphylococcus epidermidis, Strain NIHLM001 | NWP66 | PF4V1 |
| Collinsella sp., Strain 4_8_47FAA | NWP198 | PLA2G2E |
| Coprococcus sp., Strain AAb34 | AB34 | FAM19A2 |
| Propionibacterium acnes, Strain HL005PA4 | NWP220 | C3AR_Epitope-3 |
| Escherichia coli, Strain MS 145-7 | NWP33 | NAPSA |
| Escherichia coli, Strain 83972 | NWP04 | MUCL3 |
| Staphylococcus warneri, Strain SK66 | NWP11 | MFAP3 |
| Prevotella bivia, Strain DNF00650 | NWP176 | MANF |
| Streptococcus parasanguinis, Strain F0449 | NWP114 | ICAM3 |
| Proteus mirabilis, Strain WGLW6 | NWP59 | SCGB2A2 |
| Staphylococcus capitis C4 | capitisC4 | CCL18 |
| Escherichia sp., Strain 3_2_53FAA | NWP02 | ZACN |
| Collinsella sp., Strain 4_8_47FAA | NWP198 | FGF17 |
| Staphylococcus epidermidis D4 | EpiD4 | RNASE8 |
| Erysipelotrichaceae sp., Strain 6_1_45 | NWP174 | MUC7 |
| Coprococcus sp., Strain AAb34 | AB34 | PRR27 |
| Escherichia sp., Strain 3_2_53FAA | NWP02 | PRR27 |
| Staphylococcus saprophyticus D10 | saprophyticusD10 | PIP |
| Escherichia coli, Strain MS 85-1 | NWP30 | CFD |
| Proteus mirabilis, Strain WGLW6 | NWP59 | PRRG1 |
| Klebsiella oxytoca, Strain MIT 10-5242 | NWP46 | TACSTD2 |
| Lactobacillus gasseri, Strain SV-16A-US | NWP102 | GNLY |
| Lactobacillus gasseri, Strain 224-1 | NWP112 | DRAXIN |
| Escherichia coli, Strain MS 21-1 | NWP38 | KDELC1 |
| Ruminococcus gnavus, Strain NWP326 | RG109 | CCL5 |
| Bifidobacterium breve, Strain JCP7499 | NWP310 | IGFBP6 |
| Escherichia coli, Strain MS 145-7 | NWP33 | S4A4_Epitope-3 |
| Staphylococcus warneri E4 | warneriE4 | OLR1 |
| Klebsiella pneumoniae subsp. pneumoniae, Strain WGLW5 | NWP55 | CHGA |
| Enterococcus faecalis, Strain TX2137 | NWP117 | SHISA2 |
| Dorea formicigenerans, Strain AAB36 | AB36 | FAM19A2 |
| Peptoniphilus lacrimalis, Strain UPII 315-B | NWP261 | TMEM119 |
| Citrobacter freundii, Strain 4_7_47CFAA | NWP28 | LOC644613 |
| Bacteroides sp., Strain D2 | NWP192 | SMR3A |
| Klebsiella sp., Strain 4_1_44FAA | NWP21 | CHGA |
| Bacteroides sp., Strain 9_1_42FAA | NWP274 | ACKR2 |
| Staphylococcus haemolyticus B5 | haemolyticusB5 | KCNK4_Epitope-1 |
| Fusobacterium sp., Strain CM22 | NWP153 | GRM3 |
| Staphylococcus haemolyticus G2 | haemolyticusG2 | KCNK4_Epitope-1 |
| Prevotella bivia, Strain DNF00650 | NWP176 | MUCL3 |
| Plesiomonas sp., Strain HPP0020 | NWP62 | CONA1_Epitope-1 |
| Staphylococcus hominis B3 | hominisB3 | CXCL9 |
| Klebsiella sp., Strain MS 92-3 | NWP40 | SLC22A5 |
| Fusobacterium periodonticum, Strain 2_1_31 | NWP160 | CLU |
| Streptococcus sp., Strain C150 | NWP121 | SFRP4 |
| Eubacterium infirmum, Strain F0142 | NWP223 | MFSD2A |
| Staphylococcus warneri D11 | warneriD12 | KCNMB2 |
| Klebsiella oxytoca, Strain MIT 10-5246 | NWP50 | PPY |
| Clostridium difficile, Strain 050-P50-2011 | NWP278 | MFSD2A |
| Allobaculum sp., Strain NWP324 | Ery128 | GPER1_Epitope-1 |
| Proteus mirabilis, Strain WGLW6 | NWP59 | TGOLN2 |
| Peptoniphilus lacrimalis, Strain UPII 315-B | NWP261 | NKAI1_Epitope-2 |
| Escherichia coli, Strain MS 69-1 | NWP39 | ADAMTS16 |
| Bacteroides sp., Strain D2 | NWP192 | AGRP |
| Staphylococcus warneri F1 | warneriF1 | SHISA6 |
| Streptococcus salivarius, Strain SK126 | NWP118 | LMBRD2 |
| Propionibacterium acnes, Strain HL002PA1 | NWP206 | CNGB3_Epitope-3 |
| Enterococcus faecium, Strain ERV165 | NWP81 | CCL17 |
| Enterococcus faecium, Strain ERV102 | NWP80 | FGFRL1 |
| Escherichia coli, Strain MS 69-1 | NWP39 | CONA1_Epitope-1 |
| Enterococcus faecium, Strain TX1330 | NWP19 | TMEM204 |
| Streptococcus sp., Strain OBRC6 | NWP124 | ARSA |
| Staphylococcus capitis A7 | capitisA7 | FGF1 |
| Prevotella timonensis, Strain CRIS 5C-B1 | NWP156 | NGFR |
| Streptococcus salivarius, Strain SK126 | NWP118 | FGFRL1 |
| Streptococcus downei, Oral Taxon 594, Strain F0415 | NWP123 | SFRP4 |
| Propionibacterium acnes, Strain HL036PA2 | NWP227 | PDCD1 |
| Proteus mirabilis, Strain WGLW6 | NWP59 | RCN1 |
| Bacteroidales bacterium, Strain KA00344 | NWP302 | SHISA9 |
| Citrobacter freundii, Strain 4_7_47CFAA | NWP88 | SMOC2 |
| Escherichia coli, Strain MS 16-3 | NWP37 | SEMG1 |
| Prevotella bivia, Strain DNF00650 | NWP176 | CLU |
| Sporosarcina sp., Strain 2681 | NWP29 | TMEM204 |
| Staphylococcus capitis A7 | capitisA7 | FGF21 |
| Citrobacter freundii, Strain 4_7_47CFAA | NWP88 | PTHLH |
| Staphylococcus aureus, Strain TCH70 | NWP12 | IL5_sc |
| Enterococcus faecium, Strain ERV102 | NWP80 | SMR3A |
| Lactobacillus parafarraginis, Strain F0439 | NWP105 | LYVE1 |
| Staphylococcus haemolyticus G9 | haemolyticusG9 | KCNK1 |
| Lactobacillus parafarraginis, Strain F0439 | NWP105 | FAM19A3 |
| Klebsiella pneumoniae subsp. pneumoniae, Strain WGLW2 | NWP57 | NGFR |
| Lactobacillus jensenii, Strain EX849587VC06 | NWP109 | LGALS3 |
| Streptococcus intermedius, Strain F0413 | NWP128 | CTRB2 |
| Staphylococcus epidermidis, Strain NIHLM067 | NWP68 | AVP |
| Arthrobacter albus, Strain DNF00011 | NWP86 | RCN1 |
| Clostridiales sp., Oral Taxon 876, Strain F0540 | NWP280 | SHISA2 |
| Actinomyces neuii, Strain MJR8396A | NWP258 | CGA |
| Lactobacillus jensenii, Strain 269-3 | NWP110 | CD14 |
| Proteus mirabilis, Strain WGLW6 | NWP59 | REG1A |
| Escherichia coli, Strain MS 200-1 | NWP41 | PRR27 |
| Staphylococcus capitis C12 | capitisC12 | COL10A1 |
| Staphylococcus warneri F1 | warneriF1 | CSAG1 |
| Staphylococcus epidermidis, Strain NIH04008 | NWP77 | FGFRL1 |
| Escherichia coli, Strain MS 57-2 | NWP34 | DPP7 |
| Staphylococcus aureus, Strain MN8 | NWP17 | CCL5 |
| Bacteroides sp., Strain D2 | NWP192 | ADAMTS16 |
| Propionibacterium acnes, Strain HL036PA1 | NWP226 | SPINK14 |
| Eubacterium sp., Strain 3_1_31 | NWP182 | CTRB1 |
| Fusobacterium sp., Oral Taxon 370, Strain F0437 | NWP143 | TMEM149 |
| Propionibacterium acnes, Strain HL027PA1 | NWP211 | IGSF4B |
| Staphylococcus warneri D11 | warneriD12 | CSF2RA |
| Corynebacterium sp., Strain HFH0082 | NWP61 | TMEM204 |
| Arthrobacter albus, Strain DNF00011 | NWP86 | SDC3 |
| Staphylococcus haemolyticus B6 | haemolyticusB6 | KCNK4_Epitope-1 |
| Escherichia coli, Strain MS 145-7 | NWP33 | DPP7 |
| Propionibacterium acnes, Strain HL030PA1 | NWP212 | SYCN |
| Streptococcus cristatus, Strain F0329 | NWP126 | CTRB2 |
| Klebsiella oxytoca, Strain MIT 10-5245 | NWP49 | SLC6A19 |
| Staphylococcus capitis A7 | capitisA7 | OLR1 |
| Staphylococcus epidermidis, Strain NIHLM008 | NWP70 | AVP |
| Escherichia coli, Strain MS 16-3 | NWP37 | CONA1_Epitope-1 |
| Staphylococcus intermedius F7 | intermediusF7 | LOC644613 |
| Fusobacterium nucleatum, Strain CTI-07 | NWP190 | COL10A1 |
| Erysipelotrichaceae sp., Strain 6_1_45 | NWP174 | NPPA |
| Lactobacillus jensenii, Strain 269-3 | NWP110 | AXL |
| Staphylococcus aureus, Strain CM05 | NWP83 | SCN7A_Epitope-3 |
| Klebsiella pneumoniae subsp. pneumoniae, Strain WGLW2 | NWP57 | TACSTD2 |
| Escherichia coli, Strain MS 145-7 | NWP33 | SDC3 |
| Rhodococcus erythropolis, Strain SK121 | NWP95 | LOC644613 |
| Proteus mirabilis, Strain WGLW6 | NWP59 | ITPR3_Epitope-3 |
| Streptococcus mitis, Strain F0392 | NWP26 | C4A |
| Prevotella sp., Oral Taxon 472, Strain F0295 | NWP191 | MUC20 |
| Staphylococcus epidermidis, Strain NIHLM001 | NWP66 | S28A1_Epitope-6 |
| Propionibacterium acnes, Strain HL036PA2 | NWP227 | ADM2 |
| Streptococcus sp., Strain OBRC6 | NWP124 | SDF2L1 |
| Propionibacterium acnes, Strain HL005PA1 | NWP224 | GPR37 |
| Deinococcus grandis, Strain SK125 | NWP89 | LECT2 |
| Fusobacterium sp., Strain CM21 | NWP166 | SMR3A |
| Eubacterium sp., Strain AS15 | NWP254 | TMEM8B |
| Staphylococcus epidermidis D4 | EpiD4 | MUCL3 |
| Staphylococcus epidermidis, Strain NIHLM031 | NWP73 | CCL24 |
| Staphylococcus warneri B9 | warneriB9 | IL21 |
| Staphylococcus pasteuri G3 | pasteuriG3 | TMEFF2 |
| Staphylococcus epidermidis, Strain NIH04008 | NWP77 | FAM19A5 |
| Streptococcus sp., Strain OBRC6 | NWP124 | PF4V1 |
| Staphylococcus capitis A7 | capitisA7 | RTN4R |
| Escherichia coli, Strain MS 200-1 | NWP41 | AGRP |
| Bacteroides stercoris, Strain CC31F | NWP270 | CHRDL1 |
| Prevotella bivia, Strain DNF00650 | NWP176 | DEFB130B |
| Fusobacterium nucleatum, Strain CTI-07 | NWP190 | PIANP |
| Enterococcus faecium, Strain ERV165 | NWP81 | RCN1 |
| Plesiomonas sp., Strain HPP0020 | NWP62 | TNFSF9 |
| Streptococcus cristatus, Strain F0329 | NWP126 | SLC8B1 |
| Klebsiella sp., Strain 4_1_44FAA | NWP21 | AVP |
| Streptococcus mitis, Strain F0392 | NWP26 | ADAMTS16 |
| Clostridium orbiscindens, Strain 1_3_50AFAA | NWP137 | SGCA |
| Staphylococcus capitis B8 | capitisB8 | MUC7 |
| Klebsiella pneumoniae subsp. pneumoniae, Strain WGLW3 | NWP54 | GHRHR |
| Streptococcus intermedius, Strain F0413 | NWP128 | LPAL2 |
| Staphylococcus saprophyticus E8 | saprophyticusE8 | CRELD1 |
| Staphylococcus warneri D11 | warneriD12 | FAT2 |
| Collinsella sp., Strain 4_8_47FAA | NWP198 | CTRB1 |
| Collinsella aerofaciens, Strain AAB3 | AB3 | MUC7 |
| Staphylococcus saprophyticus D6 | saprophyticusD6 | TNFRSF21 |
| Escherichia coli, Strain MS 85-1 | NWP30 | CCL23 |
| Citrobacter freundii, Strain 4_7_47CFAA | NWP28 | DKK1 |
| Fusobacterium sp., Strain CM21 | NWP152 | FAM19A3 |
| Lachnospiraceae bacterium, Strain AAB26 | AB26 | SCGB1D4 |
| Lactobacillus gasseri, Strain EX33690VC16 | NWP113 | DLK1 |
| Escherichia coli, Strain MS 21-1 | NWP38 | SLC6A13 |
| Escherichia coli, Strain MS 196-1 | NWP42 | ASIP |
| Escherichia coli, Strain MS 69-1 | NWP39 | IGFBP5 |
| Dorea sp., Strain AAB35 | AB35 | FAM19A5 |
| Catabacter hongkongensis, Strain AB8_9 | NWP299 | MIA |
| Proteus mirabilis, Strain WGLW4 | NWP58 | SLC22A31 |
| Enterococcus faecium, Strain ERV102 | NWP80 | SHISA6 |
| Propionibacterium acnes, Strain HL043PA2 | NWP264 | VSTM2A |
| Klebsiella pneumoniae subsp. pneumoniae, Strain WGLW3 | NWP54 | NPPA |
| Klebsiella sp., Strain OBRC7 | NWP60 | LGALS7 |
| Fusobacterium nucleatum, Strain CTI-07 | NWP190 | IL29 |
| Lactobacillus gasseri, Strain EX33690VC16 | NWP113 | TFF3 |
| Fusobacterium nucleatum, Strain CTI-03 | NWP187 | DEFB115 |
| Klebsiella pneumoniae subsp. pneumoniae, Strain WGLW3 | NWP54 | NPPC |
| Streptococcus sp., Strain CM7 | NWP133 | TSLP |
| Capnocytophaga sp., Oral Taxon 336, Strain F0502 | NWP139 | MUCL3 |
| Klebsiella pneumoniae subsp. pneumoniae, Strain WGLW1 | NWP56 | AGRP |
| Escherichia coli, Strain MS 145-7 | NWP33 | CAC1G_Epitope-9 |
| Staphylococcus warneri F1 | warneriF1 | RTBDN |
| Corynebacterium sp., Strain HFH0082 | NWP61 | C2orf40 |
| Propionibacterium acnes, Strain HL005PA4 | NWP220 | SPINT3 |
| Blautia sp., Strain AAB30 | AB30 | NPPC |
| Proteus mirabilis, Strain WGLW4 | NWP58 | LOC644613 |
| Staphylococcus capitis A7 | capitisA7 | DPEP1 |
| Streptococcus sp., Strain SR4 | NWP130 | LOC644613 |
| Staphylococcus epidermidis F12 | EpiF12 | MUCL3 |
| Micrococcus luteus, Strain SK58 | NWP08 | SEMG2 |
| Fusobacterium sp., Strain OBRC1 | NWP154 | DEFA6 |
| Corynebacterium amycolatum, Strain SK46 | NWP07 | HGFAC |
| Corynebacterium sp., Strain CMW7794 | NWP98 | NPPC |
| Micrococcus luteus, Strain SK58 | NWP08 | UNQ9165/PRO28630 |
| Staphylococcus hominis, Strain C80 | NWP25 | SHISA6 |
| Staphylococcus aureus, Strain MN8 | NWP17 | SO1C1_Epitope-5 |
| Streptococcus sp., Strain OBRC6 | NWP124 | SSC4D |
| Klebsiella oxytoca, Strain MIT 10-5250 | NWP53 | SDC3 |
| Staphylococcus aureus, Strain CM05 | NWP83 | SHISAL1 |
| Erysipelotrichaceae sp., Strain 6_1_45 | NWP181 | IFNL2 |
| Klebsiella oxytoca, Strain MIT 10-5248 | NWP51 | PTGDS |
| Klebsiella sp., Strain OBRC7 | NWP60 | CHGA |
| Bacteroides stercoris, Strain CC31F | NWP270 | PTPRJ |
| Staphylococcus warneri D9 | warneriD9 | SOSTDC1 |
| Bacteroides sp., Strain D2 | NWP192 | C4A |
| Staphylococcus epidermidis H12 | EpiH12 | PF4V1 |
| Lachnospiraceae sp., Strain DNF00160 | NWP150 | PIANP |
| Enterococcus faecalis, Strain HH22 | NWP18 | AVP |
| Peptostreptococcus sp., Strain CC14N | NWP290 | MUCL3 |
| Staphylococcus warneri E4 | warneriE4 | MTNR1B |
| Staphylococcus epidermidis C6 | EpiC6 | CSAG1 |
| Escherichia coli, Strain MS 57-2 | NWP34 | KISS1 |
| Propionibacterium acnes, Strain HL005PA4 | NWP220 | CNGB3_Epitope-3 |
| Streptococcus parasanguinis, Strain F0449 | NWP114 | AGER |
| Escherichia coli, Strain MS 85-1 | NWP30 | CSAG1 |
| Propionibacterium acnes, Strain HL020PA1 | NWP221 | SLC22A31 |
| Arthrobacter albus, Strain DNF00011 | NWP86 | DEFB130A |
| Staphylococcus pasteuri C9 | pasteuriC9 | UNQ6190/PRO20217 |
| Streptococcus sp., Strain CM6 | NWP136 | PTPRR |
| Propionibacterium acnes, Strain HL060PA1 | NWP239 | C3AR_Epitope-3 |
| Prevotella bivia, Strain DNF00650 | NWP176 | RTN4RL1 |
| Plesiomonas sp., Strain HPP0020 | NWP62 | KLK10 |
| Escherichia coli, Strain MS 145-7 | NWP33 | PSG4 |
| Escherichia coli, Strain MS 196-1 | NWP42 | FGF17 |
| Achromobacter xylosoxidans, Strain C54 | NWP22 | AVP |
| Fusobacterium sp., Oral Taxon 370, Strain F0437 | NWP143 | CD55 |
| Eggerthella sp., Strain MVA1 | NWP185 | MUCL3 |
| Klebsiella oxytoca, Strain MIT 10-5246 | NWP50 | TMEM149 |
| Bacteroidales bacterium, Strain KA00344 | NWP302 | EPHA5 |
| Citrobacter freundii, Strain 4_7_47CFAA | NWP28 | BTC |
| Escherichia coli, Strain MS 196-1 | NWP42 | COL10A1 |
| Escherichia coli, Strain MS 200-1 | NWP41 | TMEM204 |
| Escherichia coli, Strain 83972 | NWP04 | UNQ6190/PRO20217 |
| Klebsiella oxytoca, Strain MIT 10-5250 | NWP53 | SFN |
| Streptococcus sp., Strain SR1 | NWP122 | MUCL3 |
| Escherichia coli, Strain MS 145-7 | NWP33 | CAC1E_Epitope-12 |
| Propionibacterium acnes, Strain HL036PA3 | NWP222 | CNGB3_Epitope-3 |
| Streptococcus gallolyticus subsp. gallolyticus, Strain TX20005 | NWP27 | SYCN |
| Klebsiella sp., Strain 4_1_44FAA | NWP21 | FAM174A |
| Enterococcus faecium, Strain TX1330 | NWP19 | MUCL3 |
| Propionibacterium acnes, Strain SK137 | NWP309 | TACSTD2 |
| Lactobacillus gasseri, Strain EX33690VC16 | NWP113 | CDH7 |
| Staphylococcus epidermidis, Strain NIHLM001 | NWP66 | TMEM119 |
| Staphylococcus capitis C4 | capitisC4 | MUC7 |
| Staphylococcus hominis A9 | hominisA9 | MUCL3 |
| Staphylococcus haemolytics, Strain DNF00585 | NWP87 | CTRB2 |
| Escherichia coli, Strain MS 200-1 | NWP41 | FAM19A2 |
| Propionibacterium acnes, Strain HL050PA1 | NWP248 | PDCD1 |
| Fusobacterium nucleatum, Strain CTI-02 | NWP186 | CCL17 |
| Staphylococcus saprophyticus D6 | saprophyticusD6 | SHISA6 |
| Clostridium bolteae, Strain CC43_001B | NWP245 | LCN2 |
| Staphylococcus warneri B9 | warneriB9 | PRRT1 |
| Blautia sp., Strain AAB30 | AB30 | GNLY |
| Proteus mirabilis, Strain WGLW6 | NWP59 | TM9SF3 |
| Escherichia coli, Strain MS 16-3 | NWP37 | CHGA |
| Streptococcus sp., Strain CM7 | NWP133 | PIANP |
| Streptococcus gallolyticus subsp. gallolyticus, Strain TX20005 | NWP27 | GHRHR |
| Escherichia coli, Strain MS 145-7 | NWP33 | FAM19A3 |
| Staphylococcus epidermidis, Strain E13A | NWP63 | TNFSF9 |
| Escherichia coli, Strain MS 115-1 | NWP36 | FAM19A2 |
| Staphylococcus aureus, Strain TCH70 | NWP12 | EMID1 |
| Proteus mirabilis, Strain WGLW6 | NWP59 | SDC1 |
| Corynebacterium sp., Strain CMW7794 | NWP98 | PRND |
| Proteus mirabilis, Strain WGLW4 | NWP58 | AVP |
| Bacteroides stercoris, Strain CC31F | NWP270 | VSTM2L |
| Staphylococcus lugdunensis, Strain M23590 | NWP14 | DPP7 |
| Escherichia coli, Strain MS 110-3 | NWP35 | MUC7 |
| Lactobacillus jensenii, Strain EX849587VC06 | NWP109 | SPINK9 |
| Bacteroides dorei, Strain 5_1_36/D4 | NWP244 | NCAM1 |
| Lachnospiraceae sp., Strain DNF00160 | NWP150 | SHISA6 |
| Staphylococcus haemolyticus B5 | haemolyticusB5 | SCGB1A1 |
| Dorea formicigenerans, Strain AAB36 | AB36 | PRRT1 |
| Streptococcus sp., Strain BS21 | NWP131 | CTRB2 |
| Klebsiella sp., Strain 1_1_55 | NWP03 | DLK1 |
| Staphylococcus epidermidis, Strain NIHLM001 | NWP66 | LYVE1 |
| Propionibacterium acnes, Strain HL030PA2 | NWP207 | SLC6A13 |
| Fusobacterium nucleatum, Strain CTI-05 | NWP188 | SEMG1 |
| Citrobacter freundii, Strain 4_7_47CFAA | NWP88 | AGER |
| Klebsiella sp., Strain 1_1_55 | NWP03 | AVP |
| Propionibacterium acnes, Strain HL002PA1 | NWP206 | TNFRSF7 |
| Staphylococcus capitis E12 | capitisE12 | CSAG1 |
| Streptococcus sp., Strain ACS2 | NWP115 | VASN |
| Staphylococcus warneri E4 | warneriE4 | OR1L1_Epitope-1 |
| Staphylococcus epidermidis F6 | EpiF6 | PRR27 |
| Staphylococcus saprophyticus E8 | saprophyticusE8 | F3 |
| Staphylococcus aureus, Strain CM05 | NWP83 | IL5_sc |
| Streptococcus mitis, Strain F0392 | NWP26 | RARRES2 |
| Clostridium sp., Strain MSTE9 | NWP253 | FGL1 |
| Staphylococcus hominis, Strain SK119 | NWP10 | IL27RA |
| Enterococcus faecalis, Strain TUSoD Ef11 | NWP05 | GPR25 |
| Streptococcus sp., Strain OBRC6 | NWP124 | CD97_1_2_5 |
| Proteus mirabilis, Strain WGLW6 | NWP59 | CSHL1 |
| Escherichia coli, Strain MS 85-1 | NWP30 | FAM19A2 |
| Peptoniphilus sp., Oral Taxon 386, Strain F0131 | NWP255 | RARRES2 |
| Erysipelotrichaceae sp., Strain 6_1_45 | NWP174 | C4A |
| Streptococcus cristatus, Strain F0329 | NWP126 | KLK1 |
| Staphylococcus saprophyticus E8 | saprophyticusE8 | CSF2RA |
| Coprococcus sp., Strain AAb34 | AB34 | FAM19A3 |
| Propionibacterium acidifaciens, Oral Taxon 191, Strain F0233 | NWP263 | YB043 |
| Propionibacterium acnes, Strain HL025PA1 | NWP204 | PDCD1 |
| Escherichia coli, Strain MS 69-1 | NWP39 | LECT2 |
| Veillonella sp., Oral Taxon 780, Strain F0422 | NWP268 | PDIA3 |
| Streptococcus cristatus, Strain F0329 | NWP126 | NBL1 |
| Staphylococcus warneri E6 | warneriE6 | TMC3 |
| Fusobacterium sp., Strain CM22 | NWP167 | ACKR1 |
| Lactobacillus parafarraginis, Strain F0439 | NWP105 | SSBP3-AS1 |
| Lactobacillus jensenii, Strain EX849587VC06 | NWP109 | ACP4 |
| Staphylococcus warneri B9 | warneriB9 | CXCR3 |
| Escherichia coli, Strain MS 69-1 | NWP39 | C4A |
| Fusobacterium nucleatum, Strain CTI-05 | NWP188 | AGRP |
| Fusobacterium periodonticum, Strain 2_1_31 | NWP160 | SCGB1A1 |
| Escherichia coli, Strain MS 145-7 | NWP33 | GFY_Epitope-1 |
| Klebsiella pneumoniae subsp. pneumoniae, Strain WGLW3 | NWP54 | AGRP |
| Staphylococcus warneri D9 | warneriD9 | SHISA6 |
| Proteus mirabilis, Strain WGLW4 | NWP58 | PROCR |
| Bacteroides fragilis, Strain 3_1_12 | NWP271 | CEACAM7 |
| Staphylococcus epidermidis D4 | EpiD4 | FZD1_Epitope-1 |
| Streptococcus sp., Strain OBRC6 | NWP124 | ADM2 |
| Escherichia coli, Strain MS 110-3 | NWP35 | FGF19 |
| Lactobacillus crispatus, Strain JV-V01 | NWP107 | SMOC2 |
| Clostridium innocuum, Strain 6_1_30 | NWP286 | TMEM119 |
| Propionibacterium acnes, Strain HL038PA1 | NWP235 | SHISA7 |
| Shigella sp., Strain D9 | NWP06 | DRAXIN |
| Fusobacterium nucleatum, Strain CTI-07 | NWP190 | SLPI |
| Staphylococcus warneri E4 | warneriE4 | MEPE |
| Enterococcus faecium, Strain E417 | NWP79 | BTC |
| Staphylococcus capitis D5 | capitisD5 | GPR25 |
| Klebsiella oxytoca, Strain MIT 10-5246 | NWP50 | ADRA2C |
| Streptococcus gallolyticus subsp. gallolyticus, Strain TX20005 | NWP27 | VSTM2B |
| Fusobacterium nucleatum, Strain CTI-05 | NWP188 | RCN1 |
| Bacillus sp., Strain DNF00233 | NWP90 | CXCL12 |
| Staphylococcus pasteuri F12 | pasteuriF12 | SHISA8 |
| Lactobacillus gasseri, Strain EX33690VC16 | NWP113 | AVP |
| Proteus mirabilis, Strain WGLW4 | NWP58 | DQA2_Epitope-1 |
| Ruminococcus gnavus, Strain AAB28 | AB28 | PIANP |
| Klebsiella oxytoca, Strain MIT 10-5248 | NWP51 | DEFA6 |
| Peptoniphilus sp., Oral Taxon 386, Strain F0131 | NWP255 | CCL18 |
| Staphylococcus warneri E6 | warneriE6 | FAT2 |
| Staphylococcus capitis E7 | capitisE7 | CCL18 |
| Lactobacillus reuteri, Strain CF48-3A | NWP100 | SLC8B1 |
| Clostridium cadaveris, Strain CC40_001C | NWP246 | CXCR4 |
| Eubacterium sp., Strain AS15 | NWP254 | RARRES2 |
| Lactobacillus gasseri, Strain SV-16A-US | NWP102 | BTC |
| Clostridium symbiosum, Strain WAL-14673 | NWP201 | SHISA2 |
| Prevotella veroralis, Oral Taxon 572, Strain F0319 | NWP155 | TXN |
| Enterococcus faecium, Strain ERV99 | NWP82 | PEBP4 |
| Allobaculum sp., Strain NWP324 | Ery128 | GPER |
| Corynebacterium amycolatum, Strain SK46 | NWP07 | RARRES2 |
| Lactobacillus gasseri, Strain JV-V03 | NWP106 | PIANP |
| Klebsiella sp., Strain MS 92-3 | NWP40 | APOO |
| Finegoldia magna, Strain SY01 | NWP138 | LCN2 |
| Staphylococcus pasteuri E5 | pasteuriE5 | CDSN |
| Micrococcus luteus, Strain SK58 | NWP08 | MUC7 |
| Fusobacterium nucleatum, Strain CTI-07 | NWP190 | MUC7 |
| Dorea sp., Strain AAB35 | AB35 | P4HB |
| Lachnospiraceae bacterium, Strain AAB19 | AB19 | S39A6_Epitope-1 |
| Streptococcus gallolyticus subsp. gallolyticus, Strain TX20005 | NWP27 | ITPR2_Epitope-3 |
| Fusobacterium sp., Strain CM22 | NWP153 | PDPN |
| Citrobacter freundii, Strain 4_7_47CFAA | NWP28 | PTHLH |
| Pseudomonas sp., Strain HPB0071 | NWP65 | SMOC2 |
| Propionibacterium acnes, Strain HL038PA1 | NWP235 | PDCD1 |
| Lactobacillus gasseri, Strain JV-V03 | NWP106 | SPINK9 |
| Ruminococcus sp., Strain AAB27 | AB27 | MCAM |
| Streptococcus parasanguinis, Strain F0449 | NWP114 | COL9A3 |
| Staphylococcus epidermidis, Strain NIH04008 | NWP77 | DEFB130B |
| Propionibacterium acnes, Strain HL025PA2 | NWP210 | CST6 |
| Propionibacterium acidifaciens, Oral Taxon 191, Strain F0233 | NWP263 | IL29 |
| Staphylococcus capitis B12 | capitisB12 | PLA2G12B |
| Klebsiella oxytoca, Strain MIT 10-5246 | NWP50 | PRLHR_Epitope-1 |
| Streptococcus cristatus, Strain F0329 | NWP126 | GSG1L |
| Bacteroides sp., Strain D2 | NWP192 | CSAG1 |
| Fusobacterium sp., Oral Taxon 370, Strain F0437 | NWP143 | HCG22 |
| Klebsiella sp., Strain OBRC7 | NWP60 | DEFA6 |
| Staphylococcus pasteuri G7 | pasteuriG7 | MCFD2 |
| Staphylococcus warneri F1 | warneriF1 | MMP24 |
| Bacteroides sp., Strain 4_1_36 | NWP312 | IL29 |
| Selenomonas noxia, Strain F0398 | NWP213 | COL10A1 |
| Staphylococcus warneri, Strain SK66 | NWP11 | TMEFF2 |
| Lactobacillus gasseri, Strain JV-V03 | NWP106 | PF4V1 |
| Staphylococcus epidermidis, Strain NIHLM023 | NWP75 | BPHL |
| Coprococcus sp., Strain AAb34 | AB34 | NPPC |
| Lactobacillus gasseri, Strain JV-V03 | NWP106 | LECT2 |
| Klebsiella oxytoca, Strain MIT 10-5243 | NWP47 | SCTR |
| Staphylococcus capitis E7 | capitisE7 | DEFB115 |
| Staphylococcus epidermidis, Strain NIH04008 | NWP77 | ARSA |
| Propionibacterium acnes, Strain HL005PA2 | NWP215 | GPR37 |
| Megasphaera micronuciformis, Strain DNF00954 | NWP307 | CCL13 |
| Staphylococcus capitis C11 | capitisC11 | GPR25 |
| Staphylococcus warneri A2 | warneriA2 | SLC8B1 |
| Lactobacillus gasseri, Strain JV-V03 | NWP106 | DRAXIN |
| Capnocytophaga sp., Oral Taxon 336, Strain F0502 | NWP139 | PDIA6 |
| Propionibacterium acnes, Strain HL013PA1 | NWP209 | PDCD1 |
| Staphylococcus warneri E4 | warneriE4 | CLEC18A |
| Enterococcus faecium, Strain ERV102 | NWP80 | CONA1_Epitope-1 |
| Clostridiales bacterium, Strain 4_1_37FAA | NWP285 | SHISA2 |
| Clostridium bolteae, Strain CC43_001B | NWP245 | FAM19A2 |
| Proteus mirabilis, Strain WGLW6 | NWP59 | CD97_1_2_5 |
| Staphylococcus warneri D11 | warneriD12 | CSMT1_Epitope-1 |
| Staphylococcus saprophyticus E8 | saprophyticusE8 | FCGR1B |
| Bifidobacterium breve, Strain HPH0326 | NWP289 | SMR3A |
| Propionibacterium acnes, Strain HL043PA1 | NWP303 | TMEM119 |
| Staphylococcus saprophyticus E2 | saprophyticusE2 | PIANP |
| Fusobacterium sp., Strain CM22 | NWP167 | APOO |
| Staphylococcus caprae, Strain C87 | NWP24 | RAMP3 |
| Propionibacterium acnes, Strain HL005PA1 | NWP224 | IFNA8 |
| Proteus mirabilis, Strain WGLW4 | NWP58 | LRRC25 |
| Allobaculum sp., Strain NWP324 | Ery128 | PRLHR_Epitope-1 |
| Propionibacterium acnes, Strain HL005PA2 | NWP215 | GRM5 |
| Corynebacterium sp., Strain HFH0082 | NWP61 | GNLY |
| Escherichia coli, Strain MS 115-1 | NWP36 | ADAMTS16 |
| Fusobacterium sp., Strain CM21 | NWP152 | PIANP |
| Prevotella denticola, Oral Taxon 291, Strain F0289 | NWP183 | PODXL2 |
| Escherichia coli, Strain MS 119-7 | NWP31 | NAAA |
| Escherichia coli, Strain MS 145-7 | NWP33 | FGL1 |
| Bacteroides sp., Strain D2 | NWP192 | SEMG1 |
| Erysipelotrichaceae sp., Strain 6_1_45 | NWP174 | CONA1_Epitope-1 |
| Escherichia coli, Strain MS 119-7 | NWP31 | LTB |
| Klebsiella oxytoca, Strain MIT 10-5249 | NWP52 | BTN2A3P |
| Dorea formicigenerans, Strain AAB36 | AB36 | CCER2 |
| Escherichia coli, Strain MS 145-7 | NWP33 | DEFB130B |
| Staphylococcus pasteuri F12 | pasteuriF12 | SDC4 |
| Streptococcus intermedius, Strain F0413 | NWP128 | ENDOU |
| Veillonella sp., Oral Taxon 780, Strain F0422 | NWP268 | PF4V1 |
| Clostridium sp., Strain MSTE9 | NWP253 | IFNL2 |
| Sporosarcina sp., Strain 2681 | NWP29 | GNLY |
| Lactobacillus gasseri, Strain JV-V03 | NWP106 | FGF17 |
| Staphylococcus saprophyticus D6 | saprophyticusD6 | RTBDN |
| Escherichia coli, Strain MS 16-3 | NWP37 | AGRP |
| Escherichia coli, Strain MS 115-1 | NWP36 | MUC7 |
| Klebsiella oxytoca, Strain MIT 10-5249 | NWP52 | TACSTD2 |
| Klebsiella oxytoca, Strain MIT 10-5242 | NWP46 | PVRL2 |
| Staphylococcus capitis C11 | capitisC11 | AMELY |
| Bacteroides sp., Strain AAB8 | AB8 | AZU1 |
| Streptococcus salivarius, Strain SK126 | NWP118 | TFPI2 |
| Streptococcus mitis, Strain F0392 | NWP26 | APLP1 |
| Parabacteroides merdae, Strain CL09T00C40 | NWP184 | AMY2B |
| Klebsiella oxytoca, Strain MIT 10-5242 | NWP46 | PNLIPRP1 |
| Escherichia coli, Strain MS 145-7 | NWP33 | O11G2_Epitope-1 |
| Staphylococcus aureus, Strain MN8 | NWP17 | IL5_sc |
| Staphylococcus epidermidis, Strain NIHLM003 | NWP67 | XG |
| Lactobacillus crispatus, Strain GED7749C | NWP104 | GP6 |
| Escherichia coli, Strain MS 57-2 | NWP34 | AVP |
| Staphylococcus capitis C11 | capitisC11 | PDIA2 |
| Prevotella histicola, Strain F0411 | NWP144 | TRPM6_Epitope-1 |
| Plesiomonas sp., Strain HPP0020 | NWP62 | KCNK1 |
| Enterococcus faecium, Strain ERV102 | NWP80 | SEMG1 |
| Staphylococcus warneri D11 | warneriD12 | TMC3 |
| Klebsiella oxytoca, Strain MIT 10-5246 | NWP50 | NPDC1 |
| Oscillibacter sp., Strain KLE 1745 | NWP252 | TFF3 |
| Dorea sp., Strain AAB35 | AB35 | TXNDC12 |
| Peptostreptococcus sp., Strain CC14N | NWP290 | PRR27 |
| Staphylococcus aureus, Strain CM05 | NWP83 | OLR1 |
| Fusobacterium periodonticum, Strain 2_1_31 | NWP160 | GPHB5 |
| Staphylococcus hominis, Strain SK119 | NWP10 | CTRB1 |
| Megasphaera micronuciformis, Strain DNF00954 | NWP307 | PRR27 |
| Erysipelotrichaceae sp., Strain 6_1_45 | NWP181 | CHRDL2 |
| Staphylococcus capitis, Strain SK14 | NWP09 | SHISA2 |
| Escherichia coli, Strain MS 119-7 | NWP31 | MENT |
| Streptococcus sp., Strain OBRC6 | NWP124 | CSAG1 |
| Erysipelotrichaceae sp., Strain 6_1_45 | NWP174 | CHRDL2 |
| Propionibacterium acnes, Strain HL013PA1 | NWP209 | SPINT3 |
| Streptococcus sp., Strain C150 | NWP121 | TSLP |
| Clostridium sp., Strain HGF2 | NWP293 | EPHA5 |
| Escherichia coli, Strain 83972 | NWP04 | SEMG2 |
| Staphylococcus epidermidis, Strain NIHLM040 | NWP76 | AMELX |
| Streptococcus cristatus, Strain F0329 | NWP126 | CHODL |
| Klebsiella pneumoniae subsp. pneumoniae, Strain WGLW1 | NWP56 | SHISA2 |
| Corynebacterium sp., Strain HFH0082 | NWP61 | CCL23 |
| Citrobacter freundii, Strain 4_7_47CFAA | NWP28 | CCL24 |
| Fusobacterium nucleatum subsp. animalis, Oral Taxon 420, Strain F0419 | NWP196 | DKK2 |
| Proteus mirabilis, Strain WGLW6 | NWP59 | SDF4 |
| Klebsiella oxytoca, Strain MIT 10-5249 | NWP52 | IGFBP5 |
| Propionibacterium acnes, Strain HL056PA1 | NWP267 | SLC22A31 |
| Clostridium orbiscindens, Strain CC43_001K | NWP259 | MFSD2A |
| Streptococcus sp., Strain ACC21 | NWP134 | LOC644613 |
| Propionibacterium acnes, Strain HL005PA4 | NWP220 | SSTR4 |
| Prevotella oralis, Strain HGA0225 | NWP147 | GPR151 |
| Staphylococcus epidermidis, Strain NIHLM067 | NWP68 | LTB |
| Streptococcus cristatus, Strain F0329 | NWP126 | MUCL3 |
| Staphylococcus epidermidis, Strain NIHLM020 | NWP74 | UNQ6190/PRO20217 |
| Eubacterium sp., Strain 3_1_31 | NWP182 | PIANP |
| Klebsiella sp., Strain 1_1_55 | NWP03 | DEFA6 |
| Staphylococcus capitis B8 | capitisB8 | COL10A1 |
| Plesiomonas sp., Strain HPP0020 | NWP62 | FGF17 |
| Streptococcus downei, Oral Taxon 594, Strain F0415 | NWP123 | PF4V1 |
| Staphylococcus capitis A7 | capitisA7 | CDSN |
| Veillonella sp., Oral Taxon 780, Strain F0422 | NWP268 | SLC8B1 |
| Staphylococcus hominis B3 | hominisB3 | UNQ6190/PRO20217 |
| Lactobacillus jensenii, Strain EX849587VC06 | NWP109 | SEMG2 |
| Staphylococcus aureus, Strain CM05 | NWP83 | SLC2A10 |
| Escherichia coli, Strain MS 145-7 | NWP33 | LPL |
| Staphylococcus capitis D5 | capitisD5 | FAM19A5 |
| Klebsiella sp., Strain 4_1_44FAA | NWP21 | NINJ1 |
| Staphylococcus capitis E7 | capitisE7 | SEMA6C |
| Bacteroides stercoris, Strain CC31F | NWP270 | ROBO4 |
| Escherichia coli, Strain 83972 | NWP04 | FGF17 |
| Fusobacterium periodonticum, Strain 2_1_31 | NWP160 | CNPY2 |
| Proteus mirabilis, Strain WGLW6 | NWP59 | CNPY2 |
| Streptococcus sp., Strain AS20 | NWP116 | FAM19A2 |
| Bacillus sp., Strain DNF00233 | NWP90 | GNLY |
| Streptococcus sp., Strain C150 | NWP121 | BTC |
| Propionibacterium acnes, Strain HL045PA1 | NWP265 | TMEM59L |
| Escherichia coli, Strain MS 115-1 | NWP36 | CSN3 |
| Klebsiella pneumoniae subsp. pneumoniae, Strain WGLW2 | NWP57 | IFNA5 |
| Staphylococcus capitis B12 | capitisB12 | CXCR4 |
| Streptococcus gallolyticus subsp. gallolyticus, Strain TX20005 | NWP27 | CLU |
| Clostridium cadaveris, Strain CC44_001G | NWP247 | SLC8B1 |
| Escherichia coli, Strain MS 57-2 | NWP34 | MENT |
| Pseudomonas sp., Strain HPB0071 | NWP65 | AGRP |
| Klebsiella sp., Strain MS 92-3 | NWP40 | GPR142 |
| Oscillibacter sp., Strain KLE 1745 | NWP252 | 4F2_Epitope-1 |
| Fusobacterium periodonticum, Strain 2_1_31 | NWP160 | LCN15 |
| Lactobacillus jensenii, Strain EX849587VC06 | NWP109 | GSG1L |
| Staphylococcus pasteuri C9 | pasteuriC9 | CCL11 |
| Propionibacterium acnes, Strain HL046PA2 | NWP266 | SLC22A31 |
| Klebsiella oxytoca, Strain MIT 10-5249 | NWP52 | VSTM2B |
| Staphylococcus warneri, Strain SK66 | NWP11 | SFTPA1 |
| Escherichia coli, Strain MS 16-3 | NWP37 | PTPRN2 |
| Clostridium symbiosum, Strain WAL-14673 | NWP201 | LGALS7 |
| Escherichia coli, Strain MS 115-1 | NWP36 | PROK1 |
| Lactobacillus gasseri, Strain JV-V03 | NWP106 | IGFBP5 |
| Staphylococcus capitis B1 | capitisB1 | LOC644613 |
| Prevotella melaninogenica, Strain D18 | NWP151 | SMR3A |
| Staphylococcus epidermidis F6 | EpiF6 | SMR3A |
| Bacteroides stercoris, Strain CC31F | NWP270 | SO6A1_Epitope-5 |
| Bacteroides sp., Strain D2 | NWP192 | DEFB130A |
| Propionibacterium acnes, Strain HL002PA2 | NWP214 | CST6 |
| Klebsiella sp., Strain 4_1_44FAA | NWP21 | SGCA |
| Enterococcus faecalis, Strain HH22 | NWP18 | PEBP4 |
| Propionibacterium acnes, Strain HL013PA1 | NWP209 | C3AR_Epitope-3 |
| Staphylococcus warneri B9 | warneriB9 | TMEM8 |
| Weissella cibaria, Strain F16_1 | NWP101 | SOSTDC1 |
| Enterococcus faecalis, Strain TX2137 | NWP117 | FAM174A |
| Lactobacillus jensenii, Strain 269-3 | NWP110 | IL25 |
| Peptoniphilus sp., Oral Taxon 836, Strain F0141 | NWP313 | PRR27 |
| Propionibacterium acnes, Strain HL002PA3 | NWP219 | TGFA |
| Staphylococcus warneri C5 | warneriC5 | SSBP3-AS1 |
| Staphylococcus hominis B3 | hominisB3 | CSAG1 |
| Dermabacter sp., Strain HFH0086 | NWP92 | KIR3DS1 |
| Prevotella bivia, Strain DNF00650 | NWP176 | UNQ6190/PRO20217 |
| Corynebacterium amycolatum, Strain SK46 | NWP07 | CSN3 |
| Coprococcus sp., Strain AAb34 | AB34 | OTOS |
| Streptococcus gallolyticus subsp. gallolyticus, Strain TX20005 | NWP27 | SMR3B |
| Streptococcus vestibularis, Strain F0396 | NWP132 | SOSTDC1 |
| Bacillus sp., Strain DNF00233 | NWP90 | S39A6_Epitope-1 |
| Erysipelotrichaceae sp., Strain 6_1_45 | NWP174 | DRAXIN |
| Bacteroides stercoris, Strain CC31F | NWP270 | SPINK1 |
| Lactobacillus crispatus, Strain JV-V01 | NWP107 | DEFA6 |
| Klebsiella oxytoca, Strain MIT 10-5243 | NWP47 | SMOC2 |
| Lactobacillus jensenii, Strain EX849587VC06 | NWP109 | SEMG1 |
| Enterococcus faecium, Strain E417 | NWP79 | DEFB130A |
| Staphylococcus hominis, Strain C80 | NWP25 | RTBDN |
| Bacillus sp., Strain DNF00233 | NWP90 | CHRDL2 |
| Enterococcus faecalis, Strain S613 | NWP119 | TFF3 |
| Streptococcus sp., Strain BS21 | NWP131 | TSLP |
| Clostridium cadaveris, Strain CC44_001G | NWP247 | LGALS7 |
| Propionibacterium acnes, Strain HL059PA1 | NWP250 | PDCD1 |
| Bacteroidales bacterium, Strain KA00344 | NWP302 | MMP16 |
| Streptococcus anginosus, Strain F0211 | NWP125 | TMEM59L |
| Clostridium difficile, Strain 002-P50-2011 | NWP230 | C3AR_Epitope-3 |
| Enterococcus faecium, Strain TX0133a04 | NWP44 | ALPP |
| Streptococcus sp., Strain C150 | NWP121 | CCL17 |
| Staphylococcus capitis C4 | capitisC4 | DEFB115 |
| Prevotella bivia, Strain DNF00650 | NWP176 | SPAG11B |
| Enterococcus faecalis, Strain TX2137 | NWP117 | FGFRL1 |
| Fusobacterium sp., Strain CM21 | NWP152 | IGFBP5 |
| Propionibacterium acnes, Strain HL005PA1 | NWP224 | TGFA |
| Klebsiella pneumoniae subsp. pneumoniae, Strain WGLW3 | NWP54 | CCL18 |
| Staphylococcus warneri D9 | warneriD9 | TNFRSF21 |
| Escherichia coli, Strain MS 16-3 | NWP37 | CEACAM1 |
| Propionibacterium acnes, Strain HL056PA1 | NWP267 | CST6 |
| Proteus mirabilis, Strain WGLW6 | NWP59 | TFF1 |
| Fusobacterium nucleatum, Strain CTI-05 | NWP188 | CHRDL2 |
| Capnocytophaga sputigena, Strain CC21_001D | NWP163 | SYCN |
| Escherichia coli, Strain MS 145-7 | NWP33 | FSHB |
| Clostridium difficile, Strain 050-P50-2011 | NWP278 | TXNDC12 |
| Propionibacterium acnes, Strain HL020PA1 | NWP221 | TMEM59L |
| Klebsiella oxytoca, Strain MIT 09-7231 | NWP45 | DUOXA2 |
| Staphylococcus warneri E4 | warneriE4 | TMC3 |
| Streptococcus sp., Strain OBRC6 | NWP124 | MCAM |
| Megasphaera sp., Strain DNF00872 | NWP295 | CXCR4 |
| Staphylococcus warneri D4 | warneriD4 | CTRB1 |
| Streptococcus sp., Strain C150 | NWP121 | ARSA |
| Eggerthella sp., Strain MVA1 | NWP185 | LECT2 |
| Klebsiella sp., Strain MS 92-3 | NWP40 | CHGA |
| Lactobacillus gasseri, Strain EX33690VC16 | NWP113 | FGF17 |
| Escherichia sp., Strain 3_2_53FAA | NWP02 | DPP7 |
| Staphylococcus epidermidis C3 | EpiC3 | TMG2_Epitope-1 |
| Fusobacterium nucleatum, Strain CTI-06 | NWP189 | LOC644613 |
| Streptococcus mitis, Strain F0392 | NWP26 | CCL23 |
| Staphylococcus warneri E6 | warneriE6 | ELFN1 |
| Staphylococcus capitis C12 | capitisC12 | PIANP |
| Klebsiella pneumoniae subsp. pneumoniae, Strain WGLW5 | NWP55 | PPY |
| Escherichia coli, Strain MS 16-3 | NWP37 | FZD3_Epitope-1 |
| Staphylococcus simulans F10 | simulansF10 | KCMA1_Epitope-1 |
| Staphylococcus pasteuri F11 | pasteuriF11 | TMEFF2 |
| Enterococcus faecium, Strain ERV102 | NWP80 | BTC |
| Staphylococcus caprae, Strain C87 | NWP24 | SHISA6 |
| Streptococcus mitis, Strain F0392 | NWP26 | CTRB2 |
| Propionibacterium acnes, Strain HL027PA1 | NWP211 | NINJ1 |
| Staphylococcus hominis, Strain C80 | NWP25 | SMR3A |
| Prevotella bivia, Strain DNF00650 | NWP176 | AGRP |
| Propionibacterium acnes, Strain HL053PA2 | NWP236 | BDKBR1 |
| Prevotella melaninogenica, Strain D18 | NWP151 | XG |
| Finegoldia magna, Strain GED7760A | NWP200 | EFEMP2 |
| Staphylococcus warneri C2 | warneriC2 | AGRP |
| Escherichia coli, Strain MS 85-1 | NWP30 | CCL13 |
| Klebsiella pneumoniae subsp. pneumoniae, Strain WGLW2 | NWP57 | APOO |
| Staphylococcus epidermidis, Strain NIHLM067 | NWP68 | SPOCK1 |
| Arthrobacter albus, Strain DNF00011 | NWP86 | TNFRSF4 |
| Klebsiella oxytoca, Strain MIT 10-5249 | NWP52 | AVP |
| Propionibacterium acnes, Strain HL053PA2 | NWP236 | CST6 |
| Streptococcus sp., Strain ACS2 | NWP115 | SLC8B1 |
| Enterococcus faecalis, Strain TX2137 | NWP117 | FGF17 |
| Staphylococcus capitis B12 | capitisB12 | DKK3 |
| Lactobacillus crispatus, Strain 125-2-CHN | NWP99 | MUCL3 |
| Propionibacterium acnes, Strain HL005PA2 | NWP215 | CST6 |
| Staphylococcus warneri C2 | warneriC2 | SSBP3-AS1 |
| Streptococcus gallolyticus subsp. gallolyticus, Strain TX20005 | NWP27 | GPA33 |
| Bacteroides stercoris, Strain CC31F | NWP270 | FKBP2 |
| Enterococcus faecium, Strain E417 | NWP79 | TFF3 |
| Lactobacillus jensenii, Strain 208-1 | NWP111 | SHISA6 |
| Escherichia sp., Strain 3_2_53FAA | NWP02 | AVP |
| Staphylococcus hominis, Strain C80 | NWP25 | PF4V1 |
| Staphylococcus caprae, Strain C87 | NWP24 | FAM19A3 |
| Bacteroidales bacterium, Strain KA00344 | NWP302 | AXL |
| Erysipelotrichaceae sp., Strain 6_1_45 | NWP181 | MUC7 |
| Klebsiella oxytoca, Strain MIT 10-5250 | NWP53 | DEFA6 |
| Rothia mucilaginosa, Strain CC87LB | NWP84 | MMP16 |
| Proteus mirabilis, Strain WGLW6 | NWP59 | DKK3 |
| Clostridiales bacterium, Strain 4_1_37FAA | NWP285 | KCMA1_Epitope-1 |
| Selenomonas noxia, Strain F0398 | NWP213 | SLC8B1 |
| Klebsiella oxytoca, Strain MIT 09-7231 | NWP45 | TRPM6_Epitope-1 |
| Staphylococcus warneri D11 | warneriD12 | PSG4 |
| Proteus mirabilis, Strain WGLW6 | NWP59 | SCGB1C2 |
| Streptococcus cristatus, Strain F0329 | NWP126 | IGHD-Fc-N |
| Weissella cibaria, Strain F16_1 | NWP101 | OTOS |
| Staphylococcus saprophyticus D6 | saprophyticusD6 | MUCL3 |
| Streptococcus sp., Strain CM7 | NWP133 | SHISA6 |
| Fusobacterium sp., Strain CM22 | NWP153 | MUCL3 |
| Bacteroides stercoris, Strain CC31F | NWP270 | EDDM3B |
| Proteus mirabilis, Strain WGLW6 | NWP59 | ASPRV1 |
| Fusobacterium periodonticum, Strain 1_A_54/D10 | NWP142 | TXNDC12 |
| Staphylococcus warneri E4 | warneriE4 | ADRA2C |
| Propionibacterium acnes, Strain HL043PA1 | NWP303 | SLC22A31 |
| Enterococcus faecalis, Strain TX2137 | NWP117 | RTBDN |
| Hungatella hathewayi, Strain WAL-18680 | NWP231 | ENDOU |
| Deinococcus grandis, Strain SK125 | NWP89 | COL10A1 |
| Staphylococcus warneri E6 | warneriE6 | SIGLEC6 |
| Propionibacterium acnes, Strain HL053PA1 | NWP260 | MMP16 |
| Streptococcus sp., Strain AS20 | NWP116 | C4A |
| Fusobacterium nucleatum subsp. animalis, Oral Taxon 420, Strain F0419 | NWP196 | INSL4 |
| Citrobacter sp., Strain 30_2 | NWP01 | SMOC2 |
| Lactobacillus gasseri, Strain EX33690VC16 | NWP113 | TMEM59L |
| Enterococcus faecium, Strain ERV165 | NWP81 | CSN3 |
| Streptococcus mitis, Strain F0392 | NWP26 | ASIP |
| Staphylococcus pasteuri F11 | pasteuriF11 | SDC4 |
| Megasphaera sp., Strain DNF00912 | NWP296 | CCL18 |
| Staphylococcus warneri D11 | warneriD12 | PTH1R |
| Propionibacterium acnes, Strain HL043PA1 | NWP303 | CNGB3_Epitope-3 |
| Lactobacillus jensenii, Strain EX849587VC03 | NWP108 | GP9 |
| Escherichia coli, Strain MS 119-7 | NWP31 | SYNDIG1L |
| Prevotella histicola, Strain F0411 | NWP144 | UNQ6190/PRO20217 |
| Eggerthella sp., Strain MVA1 | NWP185 | IL29 |
| Escherichia coli, Strain MS 85-1 | NWP30 | ADAMTS16 |
| Peptoniphilus sp., Oral Taxon 836, Strain F0141 | NWP313 | FAM19A3 |
| Staphylococcus capitis C11 | capitisC11 | SGCA |
| Proteus mirabilis, Strain WGLW6 | NWP59 | IFNA5 |
| Streptococcus gallolyticus subsp. gallolyticus, Strain TX20005 | NWP27 | SLURP1 |
| Enterococcus faecium, Strain ERV102 | NWP80 | LYVE1 |
| Micrococcus luteus, Strain SK58 | NWP08 | ADAMTS16 |
| Eubacterium sp., Strain 3_1_31 | NWP182 | TSLP |
| Klebsiella oxytoca, Strain MIT 10-5248 | NWP51 | LINC00527 |
| Propionibacterium acnes, Strain HL027PA2 | NWP218 | SHISA7 |
| Propionibacterium acnes, Strain HL053PA1 | NWP260 | EMID1 |
| Klebsiella sp., Strain MS 92-3 | NWP40 | KISS1 |
| Klebsiella oxytoca, Strain MIT 10-5246 | NWP50 | DLK1 |
| Staphylococcus hominis A4 | hominisA4 | SLC8B1 |
| Escherichia coli, Strain MS 85-1 | NWP30 | INSL4 |
| Lactobacillus parafarraginis, Strain F0439 | NWP105 | DEFB130B |
| Weissella cibaria, Strain F16_1 | NWP101 | SHISA6 |
| Klebsiella oxytoca, Strain MIT 10-5249 | NWP52 | DRAXIN |
| Escherichia coli, Strain MS 119-7 | NWP31 | LRRTM1 |
| Escherichia coli, Strain MS 69-1 | NWP39 | SEMG2 |
| Lactobacillus gasseri, Strain 224-1 | NWP112 | BMP7 |
| Enterococcus faecium, Strain ERV102 | NWP80 | IGFBP5 |
| Staphylococcus haemolyticus B5 | haemolyticusB5 | CD320 |
| Propionibacterium acnes, Strain HL013PA1 | NWP209 | BDKBR1 |
| Staphylococcus capitis E7 | capitisE7 | RARRES2 |
| Streptococcus sp., Strain SR4 | NWP130 | CSAG1 |
| Escherichia coli, Strain MS 16-3 | NWP37 | AGPAT2 |
| Lactobacillus gasseri, Strain 224-1 | NWP112 | PINLYP |
| Bifidobacterium angulatum, Strain F16_22 | NWP291 | PRLR |
| Staphylococcus aureus, Strain CM05 | NWP83 | NRN1L |
| Blautia sp., Strain KLE 1732 | NWP292 | FAM19A3 |
| Allobaculum sp., Strain NWP324 | Ery128 | SCGB1D1 |
| Proteus mirabilis, Strain WGLW6 | NWP59 | VM01 |
| Propionibacterium acnes, Strain HL025PA2 | NWP210 | PDCD1 |
| Lachnospiraceae sp., Strain DNF00160 | NWP150 | TSLP |
| Fusobacterium sp., Oral Taxon 370, Strain F0437 | NWP143 | TFF1 |
| Oscillibacter sp., Strain KLE 1745 | NWP252 | CCL18 |
| Proteus mirabilis, Strain WGLW6 | NWP59 | HCRTR2 |
| Enterococcus faecium, Strain ERV102 | NWP80 | AGRP |
| Propionibacterium acnes, Strain HL025PA2 | NWP210 | FAM174A |
| Prevotella bivia, Strain DNF00188 | NWP193 | BTC |
| Escherichia coli, Strain MS 119-7 | NWP31 | TMEM59L |
| Staphylococcus capitis, Strain SK14 | NWP09 | SMOC2 |
| Staphylococcus epidermidis, Strain W23144 | NWP15 | PEBP4 |
| Streptococcus gallolyticus subsp. gallolyticus, Strain TX20005 | NWP27 | INSL4 |
| Escherichia coli, Strain MS 119-7 | NWP31 | AVP |
| Fusobacterium sp., Strain CM22 | NWP153 | PSORS1C2 |
| Staphylococcus epidermidis, Strain E13A | NWP63 | SUSD6 |
| Enterococcus faecium, Strain TX0133a04 | NWP44 | RTBDN |
| Enterococcus faecalis, Strain TX2137 | NWP117 | CTRB2 |
| Erysipelotrichaceae sp., Strain 6_1_45 | NWP181 | ADAMTS16 |
| Propionibacterium acnes, Strain HL078PA1 | NWP305 | TMEM119 |
| Staphylococcus epidermidis, Strain NIHLM023 | NWP75 | INSL4 |
| Streptococcus salivarius, Strain SK126 | NWP118 | DEFB130A |
| Propionibacterium acnes, Strain HL043PA2 | NWP264 | LAYN |
| Propionibacterium acnes, Strain HL060PA1 | NWP239 | ADRA2C |
| Clostridium difficile, Strain 050-P50-2011 | NWP278 | CXCR4 |
| Rothia mucilaginosa, Strain CC87LB | NWP84 | PEBP4 |
| Erysipelotrichaceae sp., Strain 6_1_45 | NWP174 | IL29 |
| Streptococcus mitis, Strain F0392 | NWP26 | TRPM5 |
| Propionibacterium acnes, Strain HL005PA2 | NWP215 | SHISA7 |
| Shigella sp., Strain D9 | NWP06 | ADAMTS16 |
| Fusobacterium sp., Strain CM22 | NWP167 | IFITM10 |
| Klebsiella oxytoca, Strain MIT 10-5245 | NWP49 | LGALS7 |
| Escherichia sp., Strain 3_2_53FAA | NWP02 | PGF |
| Staphylococcus warneri D3 | warneriD3 | SSBP3-AS1 |
| Streptococcus sp., Strain OBRC6 | NWP124 | NPPC |
| Fusobacterium sp., Strain CM21 | NWP152 | ENDOU |
| Staphylococcus epidermidis G10 | EpiG10 | SGCA |
| Streptococcus sp., Strain BS29a | NWP129 | CTRB2 |
| Fusobacterium sp., Oral Taxon 370, Strain F0437 | NWP143 | ROBO4 |
| Staphylococcus epidermidis, Strain NIHLM031 | NWP73 | CCL8 |
| Streptococcus sp., Strain ACS2 | NWP115 | LILRA3 |
| Fusobacterium nucleatum, Strain CTI-02 | NWP186 | FAM19A2 |
| Catabacter hongkongensis, Strain AB8_9 | NWP299 | SPINK9 |
| Lachnospiraceae bacterium, Strain AAB23 | AB23 | CONA1_Epitope-1 |
| Staphylococcus capitis B12 | capitisB12 | PDGFA |
| Staphylococcus haemolyticus B6 | haemolyticusB6 | SCGB1A1 |
| Bifidobacterium longum subsp. longum, Strain 44B | NWP308 | PLA2G2E |
| Staphylococcus warneri C5 | warneriC5 | BTC |
| Erysipelotrichaceae sp., Strain 6_1_45 | NWP174 | GNLY |
| Bacillus sp., Strain DNF00233 | NWP90 | FAM19A2 |
| Weissella cibaria, Strain F16_1 | NWP101 | RTBDN |
| Clostridium sp., Strain HGF2 | NWP293 | PROM2_Epitope-1 |
| Streptococcus gallolyticus subsp. gallolyticus, Strain TX20005 | NWP27 | AVP |
| Streptococcus sobrinus, Strain W1703 | NWP85 | CXCL9 |
| Staphylococcus epidermidis, Strain NIHLM003 | NWP67 | CHGA |
| Streptococcus sp., Strain SR1 | NWP122 | MIA |
| Bacteroidales bacterium, Strain KA00344 | NWP302 | EPHA4 |
| Clostridiales bacterium, Strain 3_1_39B/D5 | NWP240 | ENSP00000381830 |
| Streptococcus mitis, Strain F0392 | NWP26 | CCL17 |
| Staphylococcus warneri E6 | warneriE6 | KCNMB2 |
| Lactobacillus jensenii, Strain 269-3 | NWP110 | MCAM |
| Escherichia coli, Strain MS 145-7 | NWP33 | NPDC1 |
| Staphylococcus pasteuri G3 | pasteuriG3 | FAM3B |
| Staphylococcus epidermidis, Strain NIHLM070 | NWP69 | AVP |
| Enterococcus faecium, Strain TX1330 | NWP19 | RARRES2 |
| Staphylococcus capitis A7 | capitisA7 | MEPE |
| Staphylococcus warneri D11 | warneriD12 | AXL |
| Staphylococcus capitis B8 | capitisB8 | DEFB130A |
| Propionibacterium acnes, Strain HL027PA1 | NWP211 | SPINT3 |
| Bacteroides sp., Strain D2 | NWP192 | CONA1_Epitope-1 |
| Staphylococcus epidermidis, Strain NIH04008 | NWP77 | TMEM119 |
| Streptococcus sp., Strain SR1 | NWP122 | AXL |
| Fusobacterium nucleatum, Strain MJR7757B | NWP257 | MERTK |
| Escherichia coli, Strain MS 196-1 | NWP42 | ADAMTS16 |
| Corynebacterium sp., Strain CMW7794 | NWP98 | MUC7 |
| Propionibacterium acnes, Strain HL045PA1 | NWP265 | LAYN |
| Staphylococcus pasteuri E5 | pasteuriE5 | SHISA8 |
| Staphylococcus epidermidis, Strain NIHLM001 | NWP66 | FAM19A5 |
| Proteus mirabilis, Strain WGLW6 | NWP59 | SPOCK2 |
| Proteus mirabilis, Strain WGLW4 | NWP58 | FGF19 |
| Escherichia coli, Strain MS 119-7 | NWP31 | SC5A3_Epitope-4 |
| Klebsiella oxytoca, Strain MIT 10-5246 | NWP50 | LYSMD4 |
| Micrococcus luteus, Strain SK58 | NWP08 | CCL22 |
| Streptococcus sp., Strain C150 | NWP121 | PEBP4 |
| Citrobacter sp., Strain 30_2 | NWP01 | CCL24 |
| Escherichia coli, Strain MS 110-3 | NWP35 | MUCL3 |
| Fusobacterium nucleatum subsp. animalis, Oral Taxon 420, Strain F0419 | NWP196 | MUCL3 |
| Propionibacterium acidifaciens, Oral Taxon 191, Strain F0233 | NWP263 | TFF1 |
| Streptococcus salivarius, Strain SK126 | NWP118 | VSTM2L |
| Streptococcus mitis, Strain F0392 | NWP26 | BTC |
| Streptococcus cristatus, Strain F0329 | NWP126 | CLDN11 |
| Staphylococcus pasteuri G7 | pasteuriG7 | TMEFF2 |
| Clostridium sp., Strain MSTE9 | NWP253 | PRR27 |
| Staphylococcus cohnii G11 | cohniiG11 | EPO |
| Propionibacterium acnes, Strain HL037PA1 | NWP208 | PDCD1 |
| Deinococcus grandis, Strain SK125 | NWP89 | CCL18 |
| Megasphaera micronuciformis, Strain DNF00954 | NWP307 | SPINK9 |
| Lactobacillus parafarraginis, Strain F0439 | NWP105 | FAM19A5 |
| Shuttleworthia sp., Strain MSX8B | NWP149 | EDEM2 |
| Klebsiella oxytoca, Strain MIT 10-5245 | NWP49 | SFN |
| Lachnospiraceae sp., Strain DNF00160 | NWP150 | CCL17 |
| Fusobacterium sp., Oral Taxon 370, Strain F0437 | NWP143 | MADCAM1 |
| Staphylococcus capitis A5 | capitisA5 | PIANP |
| Staphylococcus warneri, Strain SK66 | NWP11 | TUSC5 |
| Staphylococcus epidermidis H3 | EpiH3 | XG |
| Lactobacillus jensenii, Strain EX849587VC06 | NWP109 | DPP7 |
| Staphylococcus epidermidis, Strain NIH04008 | NWP77 | TSLP |
| Corynebacterium amycolatum, Strain SK46 | NWP07 | C6orf15 |
| Staphylococcus capitis B8 | capitisB8 | DEFB115 |
| Propionibacterium acnes, Strain HL007PA1 | NWP225 | CST6 |
| Bacillus sp., Strain DNF00233 | NWP90 | PROK1 |
| Prevotella nigrescens, Strain F0103 | NWP145 | SDC1 |
| Propionibacterium acnes, Strain HL050PA1 | NWP248 | SPINT3 |
| Prevotella bivia, Strain GED7760C | NWP233 | IGFBP7 |
| Streptococcus sp., Strain AS20 | NWP116 | LECT2 |
| Coprococcus sp., Strain AAb34 | AB34 | COL10A1 |
| Propionibacterium acnes, Strain HL038PA1 | NWP235 | SPINK14 |
| Fusobacterium nucleatum, Strain CTI-05 | NWP188 | SMR3A |
| Klebsiella oxytoca, Strain MIT 09-7231 | NWP45 | NCKX4_Epitope-1 |
| Capnocytophaga sputigena, Strain CC21_001D | NWP163 | AMY2B |
| Escherichia coli, Strain MS 115-1 | NWP36 | RARRES2 |
| Escherichia coli, Strain MS 57-2 | NWP34 | INSL4 |
| Plesiomonas sp., Strain HPP0020 | NWP62 | SEMG2 |
| Staphylococcus epidermidis, Strain NIH05001 | NWP78 | AJAP1 |
| Propionibacterium acnes, Strain HL059PA1 | NWP250 | CNGB3_Epitope-3 |
| Fusobacterium sp., Strain CM22 | NWP167 | IBSP |
| Blautia sp., Strain AAB30 | AB30 | PLA2G2E |
| Propionibacterium acnes, Strain HL037PA2 | NWP205 | PTPRN2 |
| Escherichia sp., Strain 3_2_53FAA | NWP02 | SMR3A |
| Escherichia coli, Strain MS 85-1 | NWP30 | CSN3 |
| Escherichia coli, Strain MS 110-3 | NWP35 | PF4V1 |
| Propionibacterium acnes, Strain HL002PA1 | NWP206 | BDKBR1 |
| Staphylococcus aureus, Strain TCH70 | NWP12 | OPN3 |
| Finegoldia magna, Strain SY01 | NWP138 | CTRB1 |
| Staphylococcus pasteuri G7 | pasteuriG7 | SDC4 |
| Staphylococcus capitis A7 | capitisA7 | FAT2 |
| Propionibacterium acnes, Strain HL005PA4 | NWP220 | SPINK9 |
| Staphylococcus warneri C5 | warneriC5 | SHISA6 |
| Escherichia coli, Strain MS 145-7 | NWP33 | LINC00527 |
| Citrobacter sp., Strain 30_2 | NWP01 | LOC644613 |
| Prevotella melaninogenica, Strain D18 | NWP151 | SRGN |
| Fusobacterium nucleatum, Strain CTI-07 | NWP190 | LOC644613 |
| Staphylococcus pasteuri F12 | pasteuriF12 | BTC |
| Enterococcus faecalis, Strain TUSoD Ef11 | NWP05 | LRRC38 |
| Streptococcus salivarius, Strain SK126 | NWP118 | SFRP4 |
| Staphylococcus gallinarum E9 | gallinarumE9 | MUCL3 |
| Shigella sp., Strain D9 | NWP06 | AGRP |
| Propionibacterium acnes, Strain HL110PA4 | NWP298 | SSC4D |
| Staphylococcus warneri D3 | warneriD3 | C2orf40 |
| Prevotella histicola, Strain F0411 | NWP144 | PDIA6 |
| Fusobacterium sp., Strain CM21 | NWP152 | LECT2 |
| Clostridium sp., Strain MSTE9 | NWP253 | PIANP |
| Megasphaera sp., Strain DNF00912 | NWP296 | MUCL3 |

The disclosures of each and every patent, patent application, and publication cited herein are hereby incorporated herein by reference in their entirety. While this invention has been disclosed with reference to specific embodiments, it is apparent that other embodiments and variations of this invention may be devised by others skilled in the art without departing from the true spirit and scope of the invention. The appended claims are intended to be construed to include all such embodiments and equivalent variations.

What is claimed is:

1. A composition for modulating an interaction of a host protein with an associated microbial cell, wherein the host protein and microbial cell are interacting partners selected from the group consisting of host protein and microbial cell interacting partners as set forth in Table 1.

2. The composition of claim 1, wherein the modulator is an inhibitor of the host protein selected from the group consisting of the host protein listed in Table 1.

3. The composition of claim 2, wherein the inhibitor is selected from the group consisting of a small interfering RNA (siRNA), a microRNA, an antisense nucleic acid, a ribozyme, an expression vector encoding a transdominant negative mutant, an antibody, an antibody fragment, a peptide, a chemical compound and a small molecule.

4. The composition of claim 1, wherein the modulator is an activator of the host protein selected from the group consisting of the host protein listed in Table 1.

5. The composition of claim 4, wherein the activator increases one or more of transcription and translation of the host protein selected from the group consisting of the host protein listed in Table 1.

6. The composition of claim 4, wherein the activator is selected from the group consisting of a nucleic acid, a protein, a peptide, a peptidomemetic, a chemical compound and a small molecule.

7. The composition of claim 1, wherein the interaction of the host protein with the associated microbial cell is selected from the group consisting of:
a) the interaction of Fusobacterium with an immune-modulatory protein selected from the group consisting of AGER, BTN3A3, BTNL8, C3AR, CCL5, CCR9, CD55, CD99L2, CEACAM4, CSF3, DKK1, DKK2, IL15RA, LAIR1, MADCAM1, MCAM, MERTK, NPY5R, SIRPA, SOST, TMEM149, TNFRSF10B, TNFRSF4, and TREML1; and
b) the interaction of Ruminococcus gnavus with a protein selected from the group consisting of CD7, TFF1, TFF2, and TFF3.

8. A method of modulating an interaction of a host protein with an associated microbial cell, wherein the host protein and microbial cell are interacting partners selected from the group consisting of host protein and microbial cell interacting partners as set forth in Table 1, the method comprising contacting a host cell with a composition for modulating the interaction of the host protein and the microbial cell.

9. The method of claim 8, wherein the modulator is an inhibitor of the host protein selected from the group consisting of the host protein listed in Table 1.

10. The method of claim 9, wherein the inhibitor is selected from the group consisting of a small interfering RNA (siRNA), a microRNA, an antisense nucleic acid, a ribozyme, an expression vector encoding a transdominant negative mutant, an antibody, an antibody fragment, a peptide, a chemical compound and a small molecule.

11. The method of claim 8, wherein the modulator is an activator of the host protein selected from the group consisting of the host protein listed in Table 1.

12. The method of claim 11, wherein the activator increases one or more of transcription and translation of the host protein selected from the group consisting of the host protein listed in Table 1.

13. The method of claim 11, wherein the activator is selected from the group consisting of a nucleic acid, a protein, a peptide, a peptidomemetic, a chemical compound and a small molecule.

14. A method of treating or preventing a disease or disorder in a subject in need thereof, the method comprising administering a composition for modulating the interaction of a host protein and a microbial cell to the subject, wherein the host protein and microbial cell are interacting partners selected from the group consisting of host protein and microbial cell interacting partners as set forth in Table 1.

15. The method of claim 14, wherein the modulator is an inhibitor of the host protein selected from the group consisting of the host protein listed in Table 1.

16. The method of claim 15, wherein the inhibitor is selected from the group consisting of a small interfering RNA (siRNA), a microRNA, an antisense nucleic acid, a ribozyme, an expression vector encoding a transdominant negative mutant, an antibody, an antibody fragment, a peptide, a chemical compound and a small molecule.

17. The method of claim 14, wherein the modulator is an activator of the host protein selected from the group consisting of the host protein listed in Table 1.

18. The method of claim 17, wherein the activator increases one or more of transcription and translation of the host protein selected from the group consisting of the host protein listed in Table 1.

19. The method of claim 17, wherein the activator is selected from the group consisting of a nucleic acid, a protein, a peptide, a peptidomemetic, a chemical compound and a small molecule.

20. The method of claim 14, wherein the disease or disorder is selected from the group consisting of inflammatory diseases, inflammatory bowel disease (IBD), irritable bowel syndrome (IBS), obesity, type 2 diabetes, cardiovascular disease, Alzheimer's disease, Parkinson's disease, cancer and atopy.
